(19) United States
(12) Patent Application Publication
(10) Pub. No.: US 2026/0062441 A1
(43) Pub. Date: Mar. 5, 2026

(54) TUMOR-SEEKING GLUCOSE-DENDRONS FOR THE DELIVERY OF CHEMOTHERAPEUTICS AND IMAGING AGENT TO CANCER CELLS

(71) Applicant: THE BOARD OF TRUSTEES OF THE UNIVERSITY OF ILLINOIS, Urbana, IL (US)

(72) Inventors: Jefferson Kar Fai CHAN, Savoy, IL (US); Amanda EAST, Urbana, IL (US)

(73) Assignee: THE BOARD OF TRUSTEES OF THE UNIVERSITY OF ILLINOIS, Urbana, IL (US)

(21) Appl. No.: 19/103,599

(22) PCT Filed: Aug. 21, 2023
(86) PCT No.: PCT/US2023/072563

§ 371 Date: Feb. 13, 2025

Related U.S. Application Data
(60) Provisional application No. 63/399,678, filed on Aug. 20, 2022.

(51) Int. Cl. C07H 23/00; A61K 49/00; G01N 21/64

(52) U.S. Cl. CPC C07H 23/00; A61K 49/0021; G01N 21/6428; G01N 2021/6439

(57) ABSTRACT

The attachment of glucose to drugs and imaging agents enables cancer cell targeting via interactions with GLUT1 overexpressed on the cell surface. The present technology describes a biomimetic approach for the design of a multivalent glucose moiety (mvGlu). We showcase the utility of this new group by developing aza-BODIPY-based contrast agents boasting a significant PA signal enhancement greater than 11-fold after spectral unmixing. Moreover, when applied to targeting cancer cells, effective staining could be achieved with ultra-low dye concentrations (50 nM) and compared to a non-targeted analog, the signal intensity was >1000-fold higher. Also, we employed the mvGlu technology to develop a logic-gated acoustogenic probe to detect intratumoral Cu(I), which is an emerging cancer biomarker, in a murine model of breast tumor. This application was not possible using other acoustogenic probes previously developed for copper sensing.

### RELATED APPLICATIONS

This application claims priority under 35 U.S.C. § 119(e) U.S. Provisional Patent Application No. 63/399,678, filed Aug. 20, 2022, which is incorporated herein by reference.

### BACKGROUND OF THE INVENTION

The targeted delivery of drugs and diagnostic imaging agents to a tumor site can limit off-target toxicities and nonspecific tissue distribution, respectively. A common strategy to achieve this is by appending a targeting ligand that is preferentially taken up by cancer cells relative to surrounding tissue. For instance, rapidly dividing cancer cells will upregulate certain surface proteins such as the folate receptor, which binds to and internalizes folate utilized to synthesize DNA, RNA, as well as other key building blocks for cell division. A notable example that leverages this interaction is CYTALUX™, an FDA approved folate-cyanine conjugate used for imaging-guided ovarian cancer surgery (Journal of the American Chemical Society 2020, 142 (35), 14993-15003). Likewise, the overexpression of the glucose transporter 1 (GLUT1) is essential in providing cancer cells with fuel via aerobic glycolysis. The reliance on this process for energy is known as the Warburg effect and has been exploited to deliver a variety of cargo (ChemBioChem 2013, 14 (17), 2263-2267). Of note, the attachment of glucose to a molecule imparts additional key benefits such as improved aqueous solubility. This property is of particular interest to the imaging community because dyes suitable for in vivo applications are often large, hydrophobic, and exhibit poor solubility.

Nearly all tumors overexpress GLUT1 to transport glucose into the cell to meet the high energy demands of a rapidly growing tumor. Accordingly, a multivalent would be a useful approach to target GLUT1 for treating a cancer.

### SUMMARY

The disclosed technology utilizes a biomimetic approach in which a multivalent glucose ligand is used to improve the cancer-targeting abilities of cargo (drugs and imaging agents) to tumors. Nearly all tumors overexpress GLUT1, which is used transporter used to transport glucose into the cell to meet the high energy demands of a rapidly growing tumor. The ligand we developed uses a branched moiety in which multiple copies of sugars interact with GLUT1 to increase uptake. This technology allows for large cargo such as photoacoustic dyes to become more soluble in vivo and increase targeting over the use of just one sugar.

Accordingly, this disclosure provides a compound represented by Formula I:

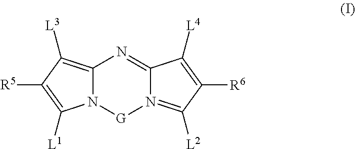

wherein
G comprises a metalloid;
L¹, L², L³, and L⁴ are independently -PhJCH₂R¹, -PhR₂, -Ph(R³)ₘ, -Ph(R⁴)ₙ, wherein Formula I includes at least one -PhJCH₂R¹;
J is O, S, or NR^{a};
R^{a} is each independently H, —(C₁-C₆)alkyl, or —(C₃-C₆) cycloalkyl;
R¹ is a substituted triazole;
R² is JCH₂R¹, H, halo, —(C₁-C₆)alkyl, —(C₃-C₆) cycloalkyl, —OR^{a}, —SR^{a}, or —N(R^{a})₂;
R³ and R⁴ are each independently H, halo, —(C₁-C₆)alkyl, —(C₃-C₆) cycloalkyl, —OR^{b}, —SR^{b}, —N(R^{b})₂, wherein R^{b} is H, —(C₁-C₆)alkyl, —(C₁-C₆)alkenyl, —(C₁-C₆)alkynyl, tris[(2-pyridyl)methyl]amine (TPA), or a metal coordination complex of TPA;
R⁵ and R⁶ are independently H, halo, —(C₁-C₆)alkyl, —(C₃-C₆) cycloalkyl, —OR^{a}, —SR^{a}, or —N(R^{a})₂; and m and n are independently 1, 2 or 3.

This disclosure also provides a method for detecting a cancer comprising:
a) contacting cancer cells and a compound of a Formula described herein in-vivo or in-vitro, wherein the compound binds to the cancer cells to form a bound compound;
b) irradiating the bound compound with near infrared (NIR) radiation; and
c) detecting a fluorescent signal or photoacoustic signal from the bound compound;

wherein a fluorescent signal or photoacoustic signal is emitted from the bound compound and the cancer is thereby detected.

The invention provides novel compounds of Formulas I, IB, II, IIB, and III, intermediates for the synthesis of compounds of Formulas I, IB, II, IIB, and III, as well as methods of preparing compounds of Formulas I, IB, II, IIB, and III. The invention also provides compounds of Formulas I, IB, II, IIB, and III that are useful as intermediates for the synthesis of other useful compounds. The invention provides for the use of compounds of Formulas I, IB, II, IIB, and III for the manufacture of medicaments useful for the treatment of bacterial infections in a mammal, such as a human.

The invention provides for the use of the compositions described herein for use in medical therapy. The medical therapy can be treating cancer, for example, breast cancer, lung cancer, pancreatic cancer, prostate cancer, or colon cancer. The invention also provides for the use of a composition as described herein for the manufacture of a medicament to treat a disease in a mammal, for example, cancer in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Absorbance of ABG and derivatives in aqueous media. Solubility of ABG with targeting ligand compared to dendrimer and dimethoxy aza-BODIPY showing ABG exhibits improved aqueous solubility.

FIG. 2. Depiction of ABG and derivatives with measured log D_{7.4} and c log P. c log P and log D₇₄ values of dyes with solubilizing groups (c log P in parentheses and log D₇₄ not in parentheses) showing ABG exhibits improved log D.

FIG. 3. Phantom images of ABG and AB-OMe₂ using spectral unmixing. PA intensity of ABG and AB-OMe₂ showing ABG exhibits improved PA unmixing.

FIG. 4. Cellular imaging with A549 lung cancer cells of ABG and derivatives. A549 lung cancer cells incubated with contrast agents showing increased cellular uptake of ABG compared to solubilized derivatives.

FIG. 5. Cellular imaging with A549 lung cancer cells to determine uptake mechanism. Uptake mechanism of ABG in A549 lung cancer cells showing ABG uptake is mediated by GLUT1.

FIG. 6. A schematic showing the targeted delivery of cargo (i.e., dye) is enhanced when a multivalent glucose targeting ligand is employed.

FIG. 7. Absorbance spectra of mvGlu-AB2, AB-Glu1, and AB-OMe₂. Spectrally unmixed PA images of b) AB-OMe₂ and c) mvGlu-AB2. d) quantified data from b) and c). Error bars=SEM. Statistical analysis was performed using a two-tailed 1-test (α=0.05, ****P<0.001).

FIG. 8. a) Representative fluorescent images of A549 human lung cancer cells treated with AB-OMe₂, mvGlu-AB2, mvGlu-AB2+glucose, or L-mvGlu-AB2. b) Quantified data from a). n=4. Error bars=SEM. Statistical analysis was performed using a two-tailed 1-test (α=0.05, **P<0.01, ***P<0.001).

FIG. 9A-E. a) Representative fluorescent image of mouse bearing 4T1 orthotopic breast cancer prior to injection (left) and following injection of mvGlu-AB2 at timepoint=16 hr (right). b) Quantified data from a) and timepoints 6, 8, 12, and 48 hrs. n=3. c) Representative PA image of tumor in cross-sectional view at timepoint=6 hr. d) Lateral view of tumor at time point=6 hr e) Quantified data from initial PA signal of tumor and c (n=4). Error bars=SEM. Statistical analysis was performed using a two-tailed 1-test (α=0.05, *P<0.01, **P<0.001***, P<0.0001****.

FIG. 10A-D. a) Normalized absorbance spectra of mvpCu (blue) and the turn-over product or t-mvpCu (red). b) Dose-dependent ratiometric turn-on of mvpCu following 1 hour incubation with 0, 25, 50, and 100 μM Cu(I) in 1:1 v/v DMF:HEPES (2 mM GSH, pH=7.4). c) Ratiometric fold turn-on following incubation of mvpCu (5 μM) with 100 μM of metal ions. d) PA tissue-mimicking phantom images of mvpCu and t-mvpCu (30 μM) in 1:1 v/v DMF:HEPES (2 mM GSH, pH=7.4) excited at the APA of mvpCu (670 nm) and t-mvpCu (765 nm). n=3. Error bars=SEM. Statistical analysis was performed using a two-tailed 1-test (*, α=0.05; **, P<0.01; ***, P<0.001).

FIG. 11A-D. a) Cartoon schematic of 4T1 tumor in cross-sectional view b) Representative PA images collected before mvpCu injection and 6 hours after. Red=t-mvpCu c) Normalized PA turn-on of t-mvpCu (n=4) d) Ratiometric fold turn-on of the turnover product of PACu-1 vs t-mvpCu at timepoints 2, 4, and 6 hours post systemic injection (n=4). Error bars=SEM. Statistical analysis was performed using a two-tailed 1-test (*, α=0.05; **, P<0.01; ***, P<0.001).

FIG. 12. A549 cells treated with low concentrations of mvGlu-AB2. Cells were incubated with mvGlu-AB2 (0, 5, 100, 500 nm) for 1 hour in serum free Ham's F-12K Medium containing 1% penicillin streptomycin. Following incubation, the dye-media solution was aspirated, and cells were washed with PBS (1×). Cells were treated with PBS and imaged using an EVOS FL epifluorescence microscope with a Cy7 filter cube. Four experimental replicates and 3 technical replicates were taken.

FIG. 13. Quantified fluorescence signal from A549 cells treated with dendron-dye conjugates: AB-OH and Glu-AB1. Cells were incubated with dye (2 μM) for 30 minutes. Media with dye solution was aspirated, and cells were washed with PBS. Cells treated with mvGluAB-2+D-Glucose were preincubated for 30 minutes with D-glucose (15 mM) prior to treatment with dye solution. Fluorescence signal is normalized to AB-OMe2 signal (n=4).

FIG. 14. 4T1 breast cancer cells treated with mvGlu-AB2. (a) Representative epifluorescence images of 4T1 cells treated with mvGlu-AB2 (2 μM), mvGluAB-2 (2 μM)+D-glucose (15 mM), or L-mvGlu-AB-2 (2 μM). Three single cell technical replicates were quantified, and data was normalized to AB-OMe₂ control (n=4). (b) Representative epifluorescence images of 4T1 cells treated with mvGlu-AB2 (2 μM), AB-Glu1 (2 μM), or AB-OH (2 μM). Three single cell technical replicates were quantified, and data was normalized to AB-OMe2 control (n=4).

FIG. 15. Cytotoxicity of mvGlu-AB2 with A549 cells. A549 cells were incubated with mvGLu-AB2 (2, 4, 6, 10, or 15 μM) for 6 hours. Viability was determined by comparing to a DMSO vehicle control (n=8).

FIG. 16. Cytotoxicity of mvGlu-AB2 with 4T1 cells. 4T1 cells were incubated with mvGlu-AB2 (2, 4, 6, 10, or 15 μM) for 6 hours. Viability was determined by comparing to a DMSO vehicle control (n=8).

FIG. 17. Fluorescence images of mvGlu-AB3. (a) Representative fluorescence images of mouse bearing 4T1 orthotopic breast cancer prior to injection and following injection of mvGlu-AB3 at timepoint=6, 8, and 12 hr. (b) Quantified data from (a) and timepoints 0, 6, 8, and 12 hours. Images were processed using FIJI (ImageJ) by using an equal ROI to determine the FLtumor/FLflank. Data is normalized relative to background signal prior to injection of dye (n=3).

FIG. 18. Fluorescence images of mvGlu-AB2 at all timepoints. Replicate fluorescence images of mice bearing 4T1 orthotopic breast cancer prior to injection and following injection of mvGlu-AB2 (1.2 mg/kg) at timepoints=6, 8, 12, 16, 24 and 48 hr. Images were processed using FIJI (ImageJ) by using an equal ROI to determine the FLtumor/FLflank. Data is normalized relative to background signal prior to injection of dye (n=3).

FIG. 19. Ratiometric fold turn-on of mvpCu. Ratiometric fold turn-on after incubation of mvpCu (5 μM) with Cu(I) (100 μM) in 1:1 v/v DMF:(50 mM) HEPES (2 mM GSH, pH=7.4) for 0, 1, 2, and 3 hours. Data normalized to t=0 (n=3).

FIG. 20. PA spectra of mvpCu and t-mvpCu. Normalized PA spectra of mvpCu and t-mvpCu (10 μM) in 1:1 v/v DMF:(50 mM) HEPES (2 mM GSH, pH=7.4).

FIG. 21. PA signal intensity of PACu-1 and mvpCu at APA. Tissue phantom images of mvpCu and PACu-1 (30 μM) in 1:1 v/v DMF:(50 mM) HEPES (2 mM GSH, pH=7.4) and quantified PA signal (n=3).

FIG. 22. Cytotoxicity of mvpCu with A549 cells. A549 cells were incubated with mvpCu (4, 6, 10, or 15 μM) for 6 hours. Viability was determined by comparing to a DMSO vehicle control (n=8).

FIG. 23. Cross-sectional PA images of mvGlu-AB2. Representative PA images of mouse bearing 4T1 orthotopic breast cancer prior to injection (left) and following injection (right) at timepoint=6 hours in cross-sectional view. Quantified data from initial and 6-hour PA signal of tumor (n=4). Error bars=SEM. Statistical analysis was performed using a two-tailed t-test (α=0.05, *P<0.01).

FIG. 24. Lateral PA images of mvGlu-AB2. Representative PA images of mouse bearing 4T1 orthotopic breast cancer prior to injection (left) and following injection (right) at timepoint=6 hours in lateral view.

FIG. 25. ICP-MS analysis of 4T1 breast tumor and healthy breast tissue. Measured Cu in excised healthy breast tissue and in the 4T1 tumor via ICP-MS.

FIG. 26. PACu-1 and mvpCu PA signal at 670 nm in vivo. Mice bearing 4T1 orthotopic breast cancer were treated with either PACu-1 or mvpCu. Tumors were quantified using equal ROIs at the APA (670 nm) for PACu-1 and mvpCu (n=4). Error bars=SEM. Statistical analysis was performed using a two-tailed t-test (α=0.05, *P<0.01, **P<0.001***, P<0.0001****).

FIG. 27. t-PACu-1 and t-mvpCu PA signal at 765 nm in vivo. Mice bearing 4T1 orthotopic breast cancer were treated with either PACu-1 or mvpCu (1.0 mg/kg in 0.9% saline (5% DMSO). Tumors were quantified using equal ROIs at the APA (765 nm) for the turnover products, t-PACu-1 and t-mvpCu (n=4). Error bars=SEM. Statistical analysis was performed using a two-tailed t-test (α=0.05, *P<0.01, **P<0.001***, P<0.0001****).

FIG. 28. 4T1 breast cancer cells treated with AB-Glu1, AB-OH, and AB-OMe2. Cells were incubated with dye (2 μM) for 30 minutes. Media with dye solution was aspirated, and cells were washed with PBS. Fluorescence signal is normalized to AB-OMe2 signal (n=4).

FIG. 29. Competition assay of mvpCu with GSH. Ratiometric fold turn-on after incubation of mvpCu (5 μM) with Cu(I) (100 μM) in 1:1 v/v DMF:HEPES (50 mM, pH=7.4) in the presence of 2 or 10 mM GSH for 1 hour (n=3).

FIG. 30. Colocalization Studies with mvGlu-AB2. Colocalization studies of mvGlu-AB2 (red) with a) DAPI (blue) b) Lysotracker Green® (green) c) MitoTracker Green® (yellow) in A549 cells. Associated merged images and qualitative scatter plots are reported. Pearson coefficients were calculated as 0.629, 0.505, and 0.475, respectively. Scale bars=100 μm.

FIG. 31. In vitro ratiometric turn on competition experiment with mvpCu and TM. Ratiometric fold turn-on after incubation of mvpCu (5 μM) with Cu(I) (50 eq) in 1:1 v/v DMF:(50 mM) HEPES (2 mM GSH, pH=7.4) or with Cu(I) (50 eq.) and tetrathiomolybdate (TM, 50 eq.) in 1:1 v/v DMF:(50 mM) HEPES (2 mM GSH, pH=7.4) for 1 hour. Data normalized to mvpCu ratiometric fold turn-on signal (n=3).

### DETAILED DESCRIPTION

The attachment of glucose to drugs and imaging agents enables cancer cell targeting via interactions with GLUT1 overexpressed on the cell surface. While an added benefit of this modification is the solubilizing effect of carbohydrates, in the context of imaging agents, aqueous solubility does not guarantee decreased π-stacking or aggregation. The resulting broadening of the absorbance spectrum is a detriment to photoacoustic (PA) imaging since the signal intensity, accuracy, and image quality all rely on reliable spectral unmixing. To address this major limitation and further enhance the tumor-targeting ability of imaging agents, we have taken a biomimetic approach to design a multivalent glucose moiety (mvGlu). We showcase the utility of this new group by developing aza-BODIPY-based contrast agents boasting a significant PA signal enhancement greater than 11-fold after spectral unmixing. Moreover, when applied to targeting cancer cells, effective staining could be achieved with ultra-low dye concentrations (50 nM) and compared to a non-targeted analog, the signal intensity was >1000-fold higher. Lastly, we employed the mvGlu technology to develop a logic-gated acoustogenic probe to detect intratumoral Cu(I), which is an emerging cancer biomarker, in a murine model of breast tumor. This exciting application was not possible using other acoustogenic probes previously developed for copper sensing.

Additional information and data supporting the invention can be found in the following publication by the inventors: Journal of the American Chemical Society 2023, 145 (13), 7313-7322 and its Supporting Information, which are incorporated herein by reference in their entirety.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N. Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of +5%, +10%, +20%, or +25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (1-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below. For example, a phenyl moiety or group may be substituted with one or more substituents RX where RX is at the ortho-, meta-, or para-position, and X is an integer variable of 1 to 5.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and l or (+) and (−) are employed to designate the sign of rotation of plane-polarized light by the compound, with (−) or l meaning that the compound is levorotatory.

The term “metalloid” refers to a type of chemical element which has properties of metals and nonmetals. Common metalloids are B, Si, Ge, As, Sb, Te, and Se. The compounds described herein comprise the metalloid, boron. The boron is bound to a pair of nitrogen atoms in the compound and therefore a positive charge exists on one nitrogen atom. The compounds described herein can exist as various salt forms.

### STATED EMBODIMENTS OF THE TECHNOLOGY

1. A compound represented by Formula I:

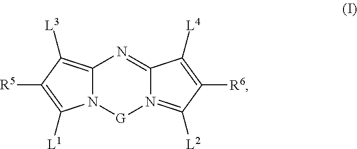

or
a salt thereof;
wherein
   G comprises a metalloid;
   L¹, L², L³, and L⁴ are independently -PhJCH₂R¹, -PhR₂, -Ph(R³)ₘ, -Ph(R⁴)ₙ, wherein Formula I includes at least one -PhJCH₂R¹;
J is O, S, or NR^{a};
R^{a} is each independently H, —(C₁-C₆)alkyl, or —(C₃-C₆) cycloalkyl;
R¹ is a substituted triazole;
R² is JCH₂R¹, H, halo, —(C₁-C₆)alkyl, —(C₃-C₆) cycloalkyl, —OR^{a}, —SR^{a}, or —N(R^{a})₂;
R³ and R⁴ are each independently H, halo, —(C₁-C₆)alkyl, —(C₃-C₆) cycloalkyl, —OR^{b}, —SR^{b}, —N(R^{b})₂, wherein R^{b} is H, —(C₁-C₆)alkyl, —(C₁-C₆)alkenyl, —(C₁-C₆)alkynyl, tris[(2-pyridyl)methyl]amine (TPA), or a metal coordination complex of TPA;
R⁵ and R⁶ are independently H, halo, —(C₁-C₆)alkyl, —(C₃-C₆) cycloalkyl, —OR^{a}, —SR^{a}, or —N(R^{a})₂; and m and n are independently 1, 2 or 3.

In some embodiments, the compound of Formula I is represented by Formula IB:

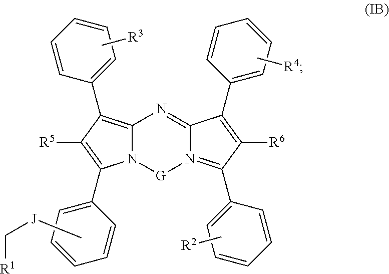

or
a salt thereof;
wherein
G comprises a metalloid;
J is O, S, or NR^{a};
R^{a} is each independently H, —(C₁-C₆)alkyl, or —(C₃-C₆) cycloalkyl;
R¹ is a substituted triazole;
R² is JCH₂R¹, H, halo, —(C₁-C₆)alkyl, —(C₃-C₆) cycloalkyl, —OR^{a}, —SR^{a}, or —N(R^{a})₂; and
R³, R⁴, R⁵ and R⁶ are independently H, halo, —(C₁-C₆)alkyl, —(C₃-C₆) cycloalkyl, —OR^{a}, —SR^{a}, or —N(R^{a})₂.
2. The compound of embodiment 1 wherein G is BX₂ wherein X is halo or alkoxy.
3. The compound of embodiment 1 or 2 wherein the substituted triazole comprises one or more saccharides, polyols, or a combination thereof.
4. The compound of embodiment 3 wherein the one or more saccharides comprise glucose.
5. The compound of embodiment 3 wherein the one or more polyols comprise glycerol.
6. The compound of any one of embodiments 1-5 represented by Formula II, IIB, or III:

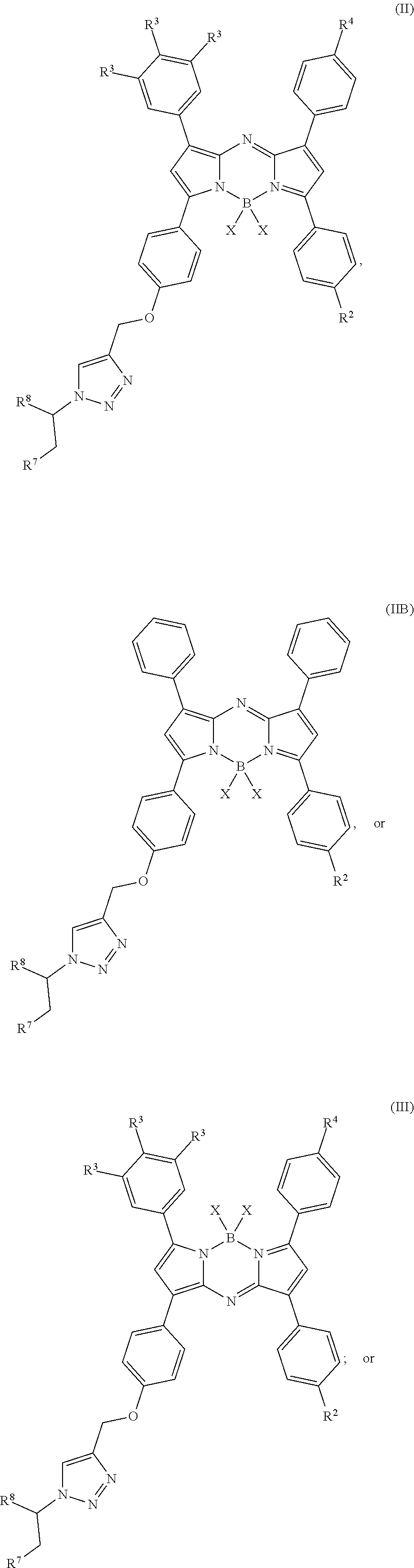

a salt thereof;
wherein
   R¹ comprises a hexose or an acyclic triol;
   R⁸ is —CH₂R⁷ or H; and
   X is fluoro, chloro, bromo, iodo, methoxy, ethoxy, or propoxy.
7. The compound of any one of embodiments 1-6 wherein X is fluoro or methoxy.
8. The compound of any one of embodiments 1-7 wherein R² is:

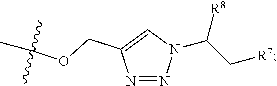

wherein
   R⁷ comprises a hexose or an acyclic triol; and
   R⁸ is —CH₂R⁷ or H.
9. The compound of any one of embodiments 1-8 wherein R³ of the phenyl group is at the position para to the pyrrole moiety of Formula I, Formula IB, Formula II or Formula III is:
H, OH, —OCH₃,

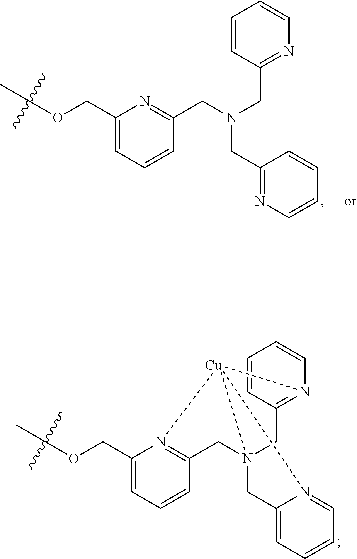

and/or
R³ of the phenyl group is at the position meta to the pyrrole moiety of Formula I, Formula IB, Formula II or Formula III are independently H or chloro.
10. The compound of any one of embodiments 1-8 wherein R⁴ is —OCH₃.
11. The compound of any one of embodiments 1-10 wherein R⁷ is:

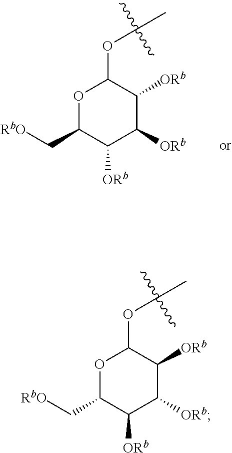

wherein each R^{b} is independently H, —(C₁-C₆)alkyl, or —CO(C₁-C₆)alkyl. In any one embodiment R⁷ comprises D-glucose or L-glucose.
12. The compound of any one of embodiments 1-11 wherein R⁷ is:

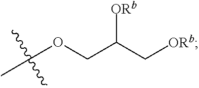

wherein each R^{b} is independently H, —(C₁-C₆)alkyl, or —CO(C₁-C₆)alkyl. In any one embodiment, R⁷ comprises glycerol.
13. The compound of any one of embodiments 6-12 wherein R⁸ is —CH₂R⁷.
14. The compound of embodiment 1 or 6 wherein the compound is ABG, mvGlu-AB1, mvGlu-AB2, mvGlu-AB3, mvpCu, mvpCu-Cu^{t}, t-mvpCu, L-ABG, mono-ABG, AB-Dendrimer, mono-AB-Dend, or a salt thereof.
15. A method for detecting a cancer comprising:
   a) contacting cancer cells and a compound of any one of embodiments 1-14 in-vivo or in-vitro, wherein the compound binds to the cancer cells to form a bound compound;
   b) irradiating the bound compound with near infrared (NIR) radiation to cause activation of the compound; and
   c) detecting a fluorescent signal or photoacoustic signal from the bound compound;
wherein a fluorescent signal or photoacoustic signal is emitted from the bound compound and the cancer is thereby detected.
16. The method of embodiment 15 wherein the compound is ABG, mvGlu-AB3, or mvpCu.
17. The method of embodiment 15 or 16 wherein the compound has a concentration in the cancer cells of about 5 nM to about 500 nM. In some embodiments, the concentration is about 50 nM. In some other embodiments, the concentration is about 20 nM, about 40 nM, about 60 nM, about 80 nM, about 100 nM, about 150 nM, about 200 nM, about 250 nM, about 300 nM, about 350 nM, about 400 nM, about 450 nM, about 500 nM, about 750 nM, or about 1000 nM.
18. The method of any one of embodiments 15 to 17 wherein the NIR radiation is at a wavelength of about 600 nm to about 800 nm. In some embodiments, the wavelength is about 670 nm (e.g., for mvpCu) or about 765 nm (e.g., for t-mvpCu). In some other embodiments, the wavelength is about 550 nm, about 600 nm, about 650 nm, about 700 nm, about 750 nm, about 800 nm, or about 850 nm.

### Description of the Technology

Biological processes have served as important sources of inspiration for the development of new technologies. Interactions between cellular receptors and ligands and the overexpression of receptors have been leveraged to selectively deliver cargo (drugs and imaging agents) to cancer cells. For example, folate, RDG, and glucose have all been used for these purposes to target the rapid metabolism of cancer cells. However, these applications only use one copy of the ligand to exploit the delivery of their cargos. An alternative strategy is to employ a powerful multivalent approach in which multiple copies of a ligand can be presented to increase binding interactions and uptake.

Molecular imaging can enable real-time visualization of complex biological processes. Photoacoustic (PA) imaging is a promising modality to allow access to high resolution imaging of biological processes in vivo. PA imaging can be summarized as a “light-in, sound out” approach in which absorbed light by a chromophore is converted to ultrasound via the PA effect.

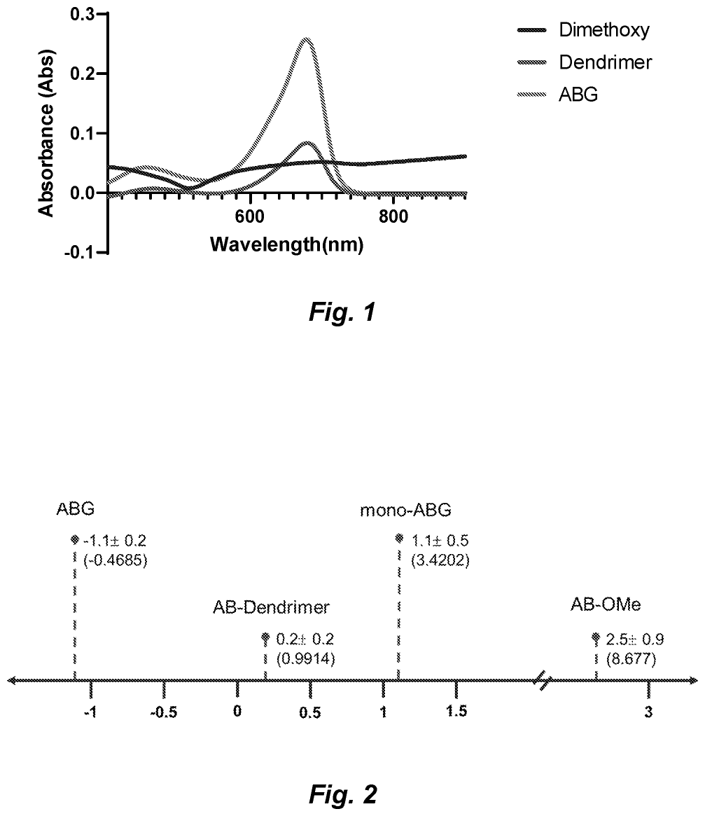

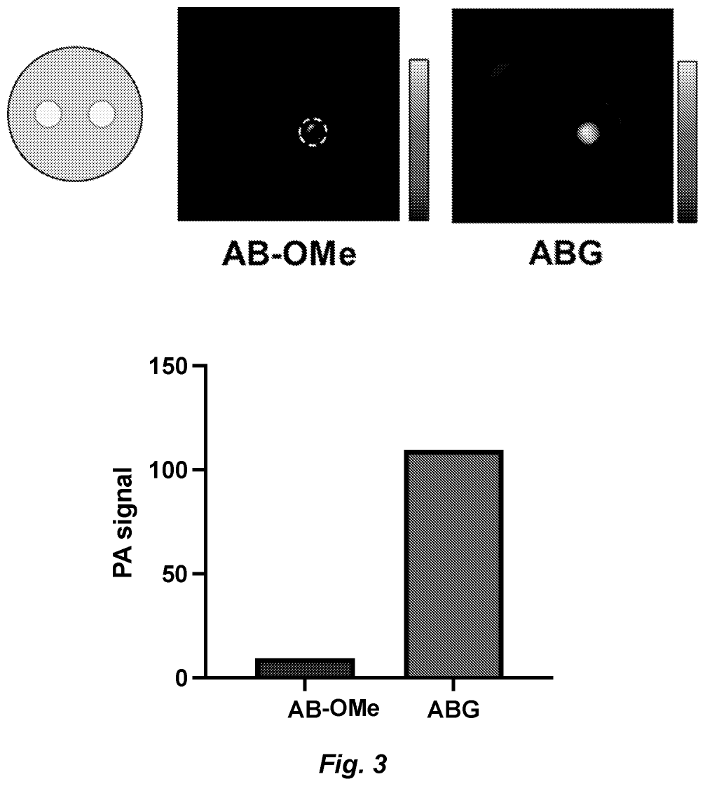

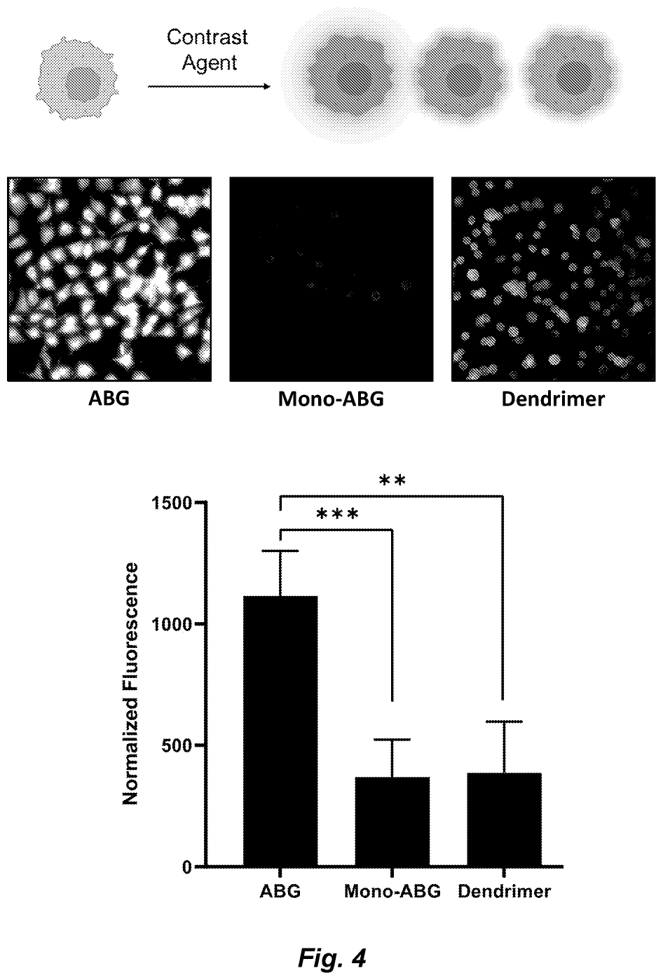

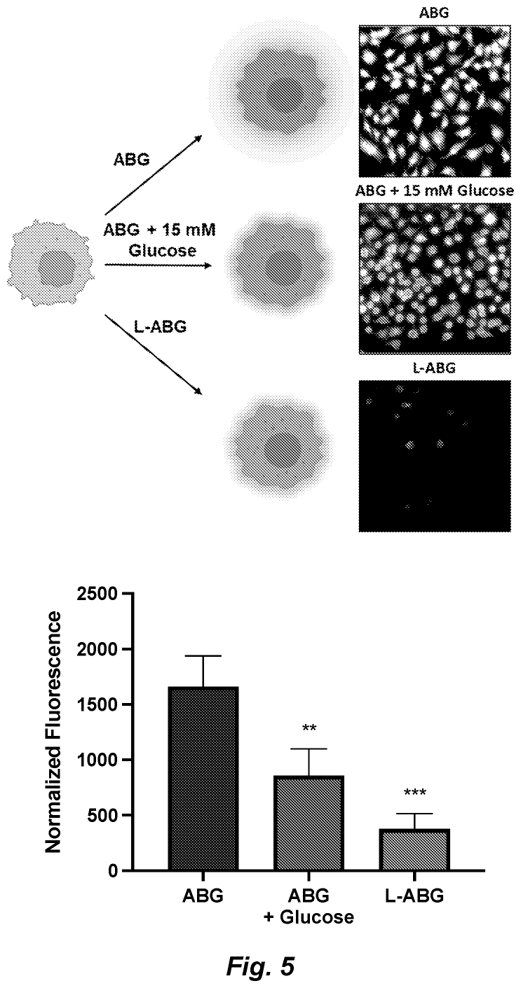

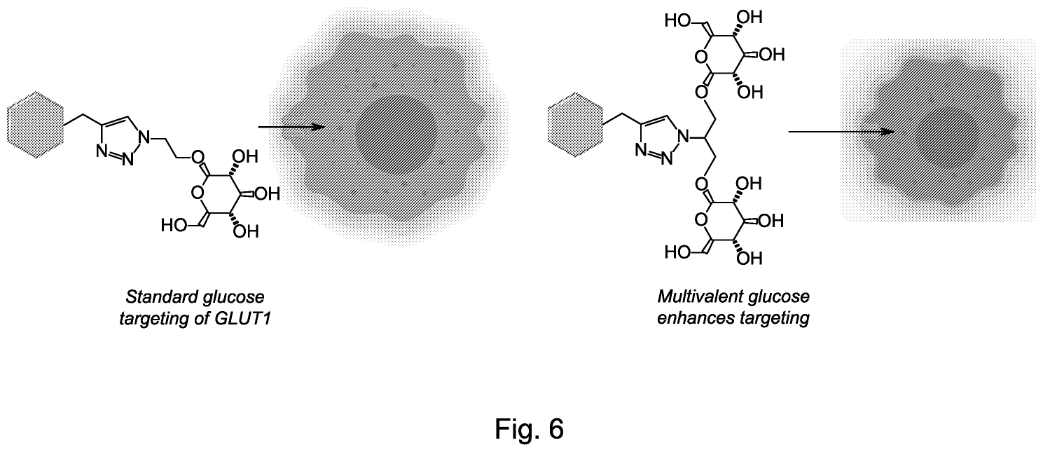

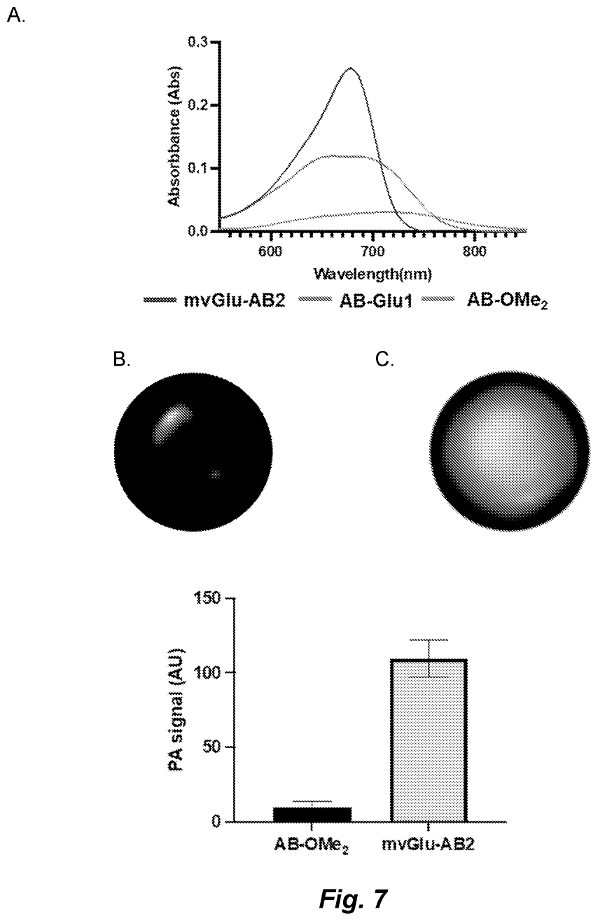

Disclosed herein is a multivalent ligand design for near-infrared (NIR) dyes to improve 1) solubility in aqueous media, 2) dye aggregation, 3) dye brightness, 4) log D, 5) PA unmixing, and 6) selective uptake in cancer cells (FIGS. 1-5).

The disclosed technology utilizes a biomimetic approach in which a multivalent glucose ligand is used to improve the cancer-targeting abilities of cargo to tumors. Nearly all tumors overexpress GLUT1, which is used transporter used to transport glucose into the cell to meet the high energy demands of a rapidly growing tumor. The ligand we developed uses a branched moiety in which multiple copies of sugars interact with GLUT1 to increase uptake. This technology allows for large cargo such as photoacoustic dyes of Formula I to become more soluble in vivo and increase targeting over the use of just one sugar.

We appended a multivalent targeting ligand to the aza-BODIPY platform to improve its solubility and cancer targeting ability (Chart 1 and Chart 2). By using a multivalent approach, we were able to increase cancer cell uptake and observe accumulation in a murine cancer model. Further directions entail highlighting the utility of this ligand through the development of an activity-based probe. Additionally, this ligand can be applied to NIR-II dyes as well.

### Chart 1. ABG and Derivatives to Compare Solubility and Cancer Targeting.

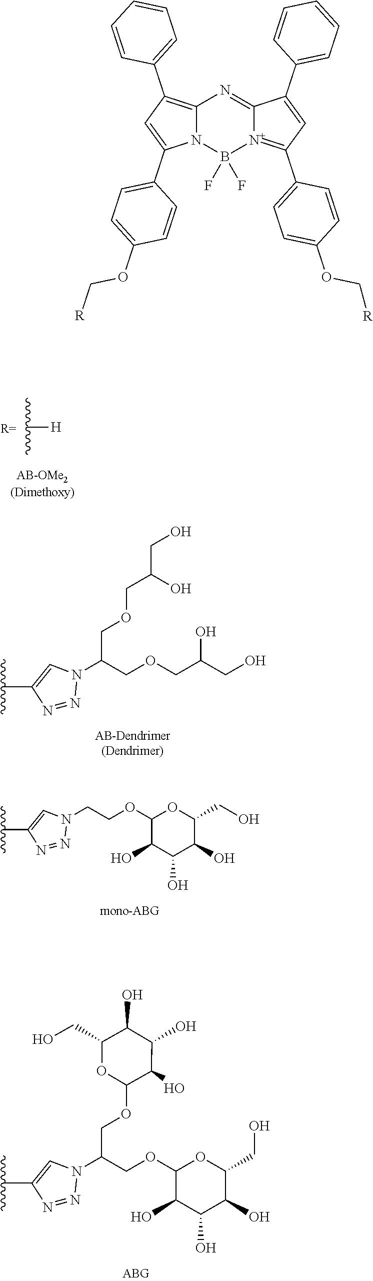

### Chart 2. Synthesis of ABG and Multivalent Ligand 3.

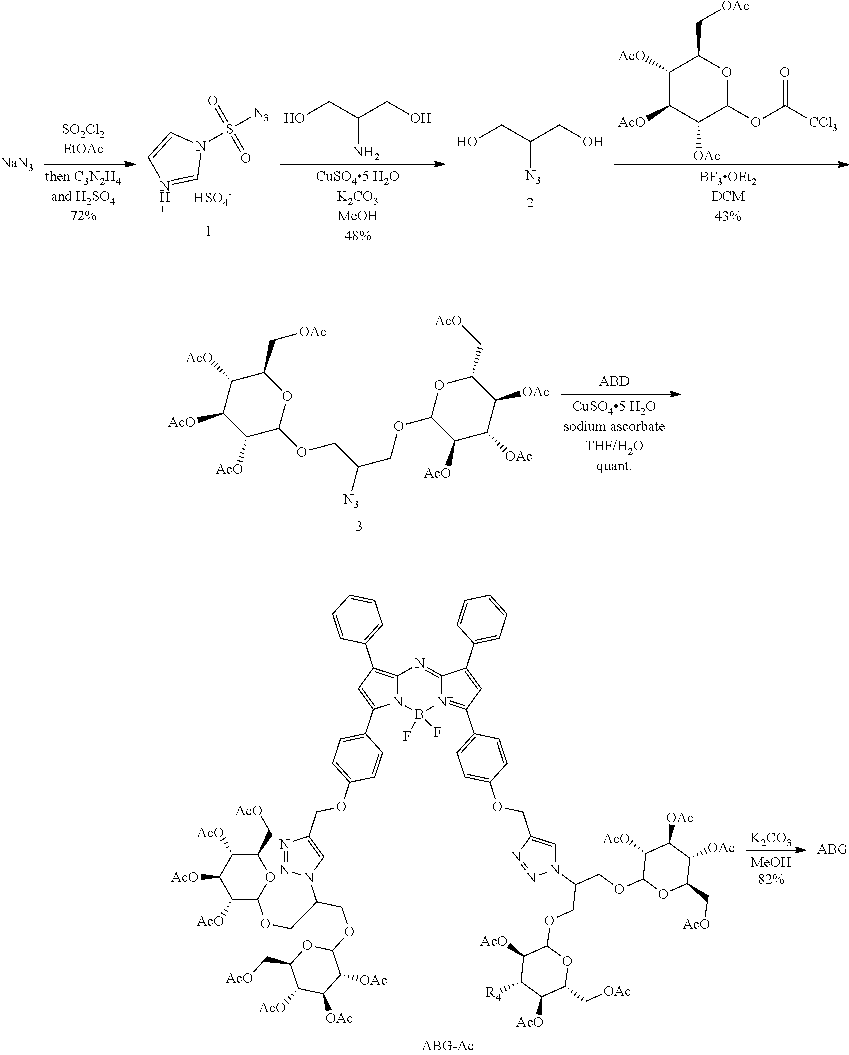

A Biomimetic Approach To Promote Cellular Uptake And Enhance Photoacoustic Properties Of Tumor-Seeking Dyes. Our group and others have contributed to the development of activity-based sensing (ABS) probes designed to monitor cancer biomarkers such as hypoxia, reactive oxygen and nitrogen species, enzymes, and thiols via photoacoustic (PA) imaging. This modality is best described as a “light-in, sound out” approach in which absorbed light is converted to ultrasound via the PA effect. Because sound can readily propagate through tissue, it is possible to obtain high resolution images in deep regions of the body. However, the quality (and accuracy) of a PA image relies on a well-resolved in vivo PA spectrum of the imaging agent to deconvolute its signal from that of background. Unfortunately, the spectra of aggregation-prone dyes, which encompass many PA imaging agents, are typically broad, spectrally shifted, and poorly defined. We attempted to address this by installing 2 glucose residues onto an aza-BODIPY (AB) dye to yield AB-Glu1 (vide infra). While we were able to enhance aqueous solubility, only moderate improvements to the PA spectra were observed, presumably because the dyes are still able to π-stack. This is consistent with reports that have demonstrated that the inclusion of strategically positioned functional groups such as oligoglycerol dendrons, quaternary ammonium centers or PEG shielding arms are required to block dye aggregation.

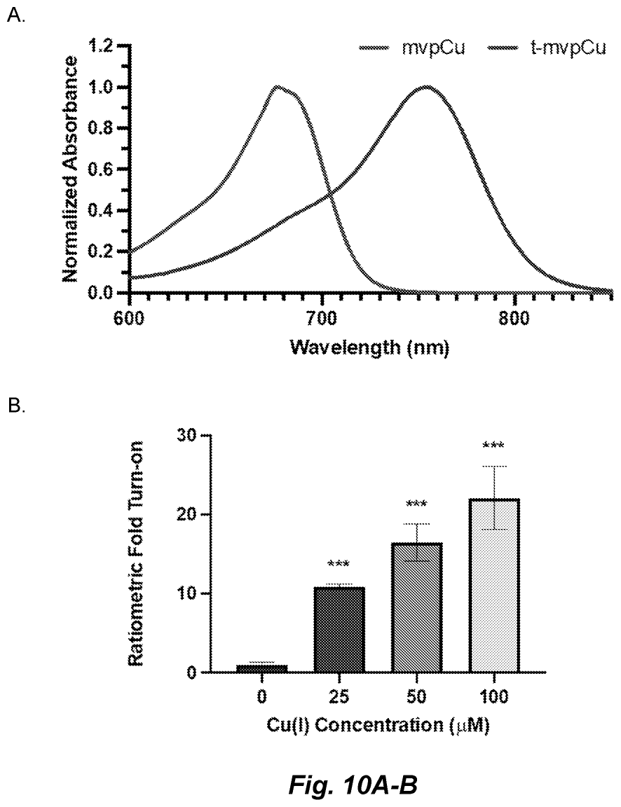

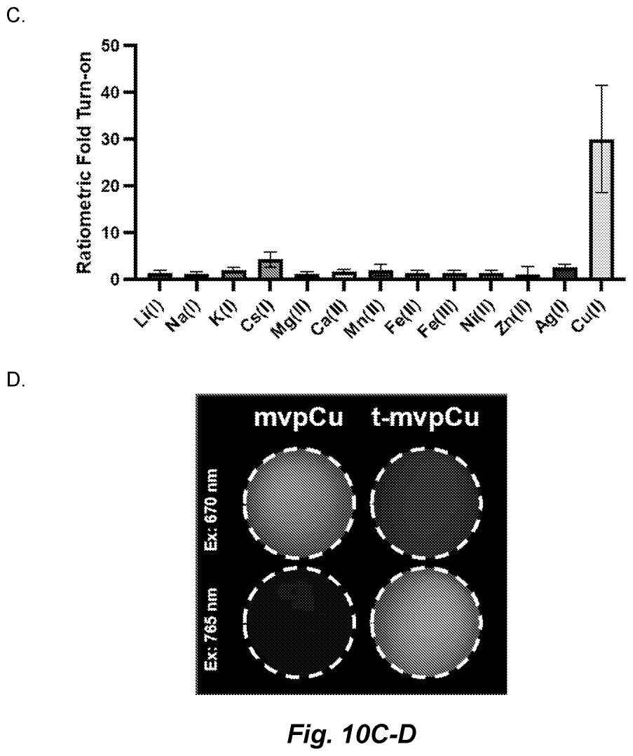

In the present study, we developed a new multivalent glucose targeting moiety (herein named mvGlu) by mimicking Nature's solution to weak binding interactions, which is to display numerous copies of a ligand to maximize receptor contact. mvGlu was designed to: 1) solubilize and 2) prevent T-stacking of the appended dye; and 3) enhance cancer targeting relative to a single glucose residue (FIG. 10). Using this new aggregation blocking and tumor-seeking motif, we prepared a panel of AB-based contrast agents for in vivo PA cancer imaging. Moreover, we developed a logic-gated acoustogenic probe by integrating this technology to detect elevated labile copper (Cu) in a murine model of breast cancer.

### Results and Discussion

Design of mvGlu-based Contrast Agents. We hypothesized the attachment of two glucose units to a glycerol scaffold through the primary alcohols would afford a branched configuration that can effectively attenuate π-stacking interactions, while simultaneously maximizing interactions with GLUT1. We chose to connect glucose through the anomeric center (as opposed to the C6 position) because this would lock the sugar into its pyranose form to increase the number of hydroxy groups available for hydrogen bonding with GLUT1. Furthermore, modification of the Cl position has been shown to tolerate large cargo without decreasing GLUT1 interaction (Biochemical and Biophysical Research Communications 2016, 474 (2), 240-246). We opted to install an azido group to facilitate attachment to alkynylated dyes (or other cargo) via click chemistry. We prepared a panel of four AB dyes comprised of 0, 1, or 2 units of mvGlu. Additionally, we varied the position (i.e., bottom versus top hemispheres of the dye) to determine the corresponding impact on aggregate formation. The chemical structures of the non-glycosylated control AB (AB-OMe₂), mvGlu-AB1, mvGlu-AB2, and mvGlu-AB3 are shown in Table 1a. Synthetic details, including full characterization are described in the Examples.

TABLE 1
a) Chemical structures of AB-OMe₂, mvGlu-AB1, mvGlu-AB2, and mvGlu-AB3. b)
Summary of photophysical properties including λ_{abs}, λₑₘ, ε, Φ, PABF, and logD_{7.4} of AB-OMe₂,
mvGlu-AB1, mvGlu-AB2, and mvGlu-AB3.
a)

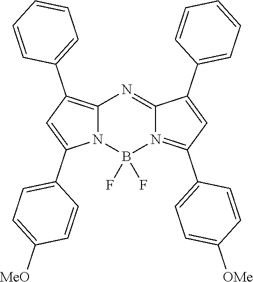

AB-OMe₂

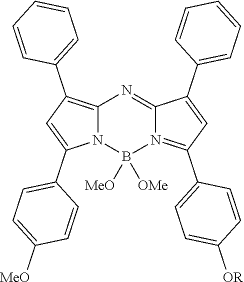

mvGlu-AB1

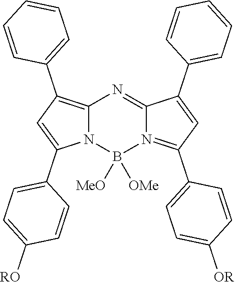

mvGlu-AB2

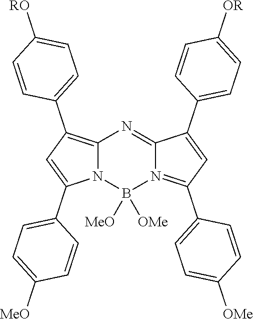

mvGlu-AB3

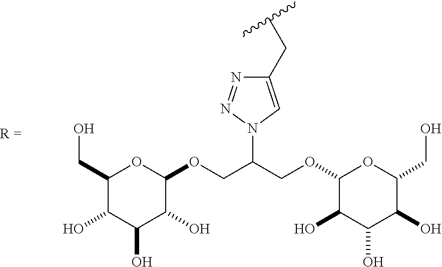

b)

| | λ_{abs} | λₑₘ | ε (×10⁴M cm⁻¹) | Φ | PABF (×10⁴) | logD_{7.4} |
|---|---|---|---|---|---|---|
| AB-OMe₂ | 689 | 710 | 0.34 | 0.014 | 0.34 | 2.51 ± 0.92 |
| mvGlu-AB1 | 675 | 710 | 1.8 | 0.10 | 1.6 | 0.75 ± 0.02 |
| mvGlu-AB2 | 679 | 710 | 2.6 | 0.19 | 2.1 | −1.12 ± 0.16 |
| mvGlu-AB3 | 681 | 712 | 2.4 | 0.31 | 1.7 | −0.71 ± 0.11 |

TABLE 2
Further photophysical properties of mvGlu-dye conjugates.

| | λ_{abs} | λₑₘ | ε(×10⁴ M cm⁻¹) | Φ | PABF × 10⁴ | logD_{7.4} | clogP |
|---|---|---|---|---|---|---|---|
| AB-OMe₂ | 689 | 710 | 0.34 | 0.014 | 0.34 | 2.51 ± 0.92 | 8.677 |
| mvGlu-AB1 | 675 | 710 | 1.8 | 0.10 | 1.6 | 0.75 ± 0.02 | 5.8485 |
| mvGlu-AB2 | 679 | 710 | 2.6 | 0.19 | 2.1 | −1.12 ± 0.16 | 0.6260 |
| mvGlu-AB3 | 681 | 712 | 2.4 | 0.31 | 1.7 | −0.71 ± 0.11 | 0.4640 |
| L-mvGlu-AB2 | 678 | 710 | 2.3 | 0.15 | 2.0 | −1.04 ± 0.53 | 0.6260 |
| AB-Glu1 | 678 | 710 | 0.78 | n.d. | n.d. | n.d. | 4.0935 |
| AB-OH | 679 | 712 | 1.3 | 0.15 | 2.0 | 0.21 ± 0.48 | 1.2434 |

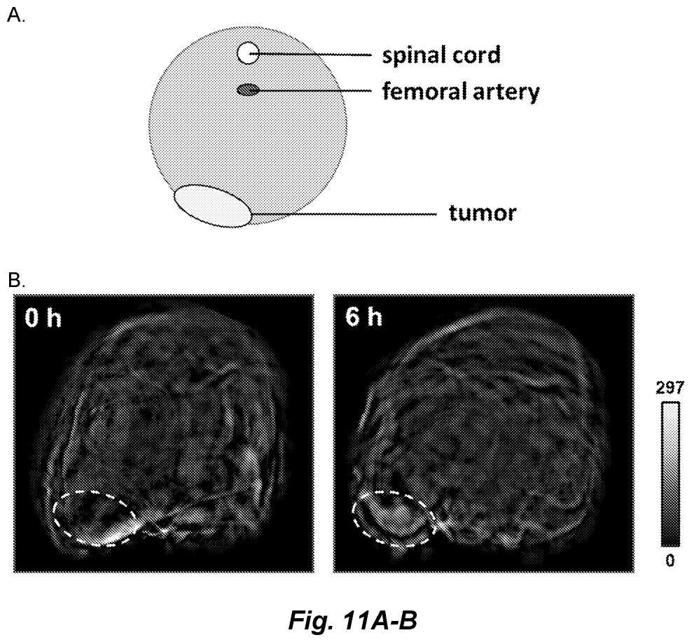

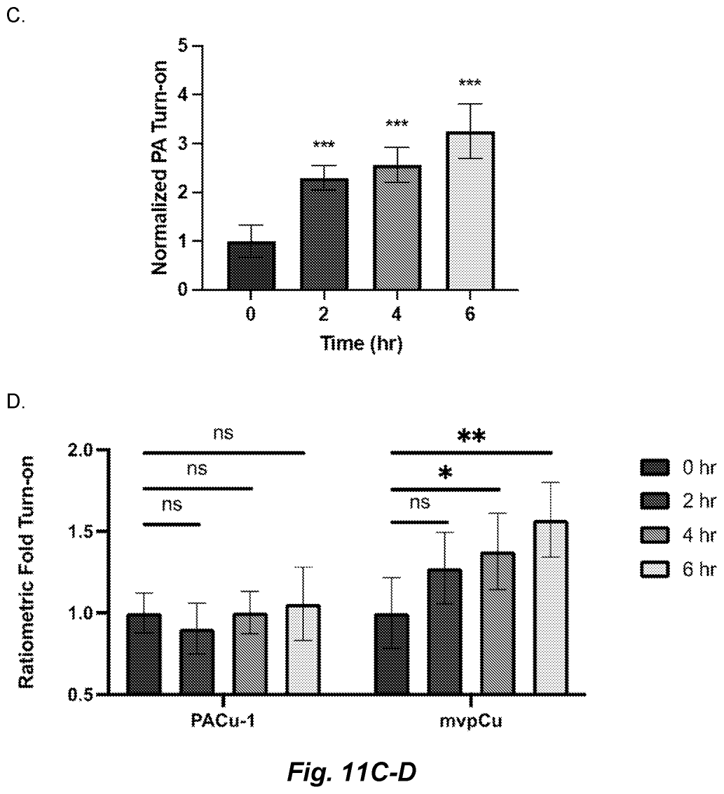

In vitro Characterization of mvGlu-dye Conjugates. With the mvGlu-AB series in hand, we began by determining the experimental octanol-water partition coefficients at pH 7.4 (log D_{7.4}). The measured log D_{7.4} values in ascending order (most to least water soluble) are: mvGlu-AB2 (−1.12±0.16), mvGlu-AB3 (−0.71±0.11), mvGlu-AB1 (0.75±0.02), and AB-OMe₂ (2.51±0.92) (Table 1b). These results indicate the installation of a single mvGlu unit already has a dramatic effect on aqueous solubility at micromolar concentrations and the inclusion of a second mvGlu group can further shift dye distribution from octanol to favor water. Next, we obtained the absorbance spectrum of each dye (3:7 v/v acetonitrile:PBS). The peak appearance of a dye is an excellent indicator of its aggregation status (FIG. 11a). For instance, owing to the broad spectrum of AB-OMe₂, it is likely to be aggregated and thus, we would anticipate spectral unmixing to be sub-optimal and the corresponding PA signal to be weak. However, when two mvGlu groups are present (regardless of the position), the peak shape is sharp, well defined, and resembles that of AB-OMe₂ in a pure organic solvent such as DMSO. In addition to improving spectral unmixing, a sharp peak is also associated with a larger extinction coefficient (8) value.

According to PA Brightness Factor (PABF) calculations, a large & is the most influential property that determines the strength of a PA signal. Because the & for mvGlu-AB2 (2.6×10⁴ M⁻¹ cm⁻¹) is 8.7-fold higher than AB-OMe₂ (0.3×10⁴ M⁻¹ cm⁻¹), we anticipate this will translate into a substantially stronger PA signal. To test this property experimentally, we cast a tissue-mimicking phantom using agar and milk. Samples of AB-OMe₂ or mvGlu-AB2 in water were then dosed with hemoglobin (10 mg/mL), embedded in the phantom, and imaged using a multispectral optoacoustic tomography (MSOT) imaging system (FIGS. 11b and 11c). Hemoglobin at such high concentrations was added because it is the major endogenous PA-active pigment that causes interference during in vivo imaging. The PA signal of AB-OMe₂ was barely above background after unmixing; however, the corresponding PA intensity of mvGlu-AB2 was 11-fold higher at the same dye concentration (FIG. 11d). Compared to representative strategies we have devised previously including conformational restriction (˜3-4-fold) and steric relaxation (˜4-fold) mvGlu is the most successful approach to date (Scheme 1).

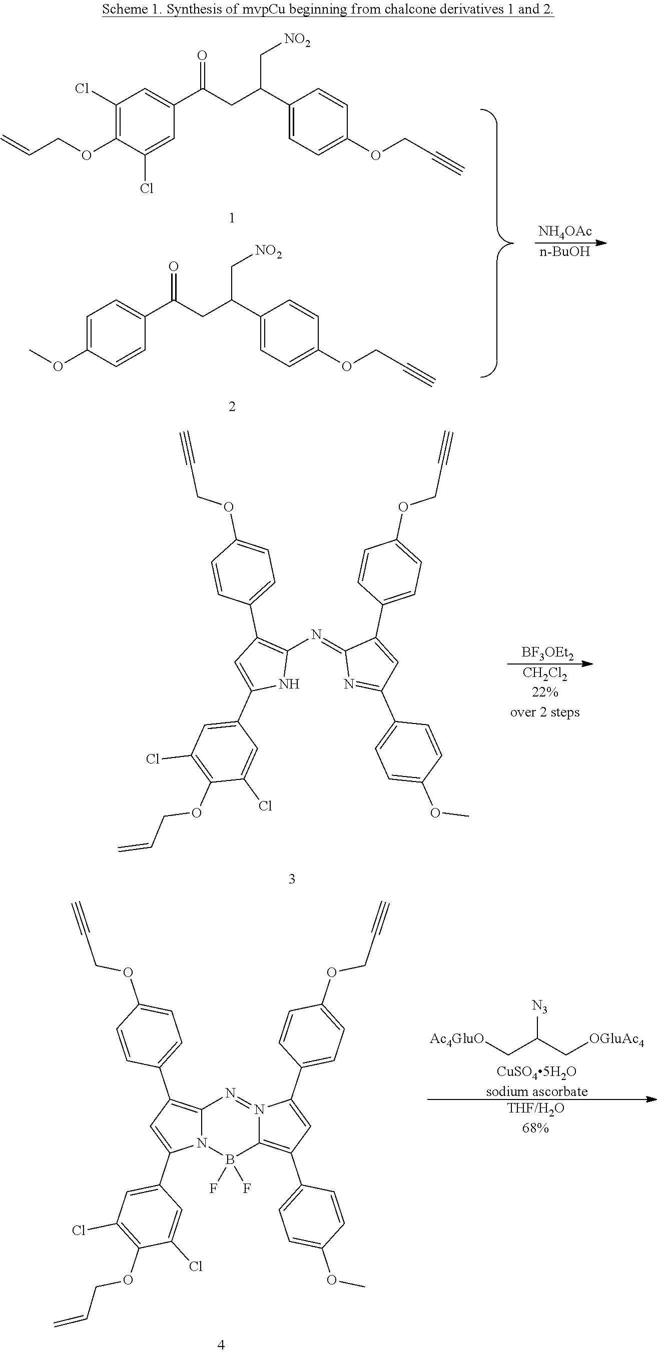

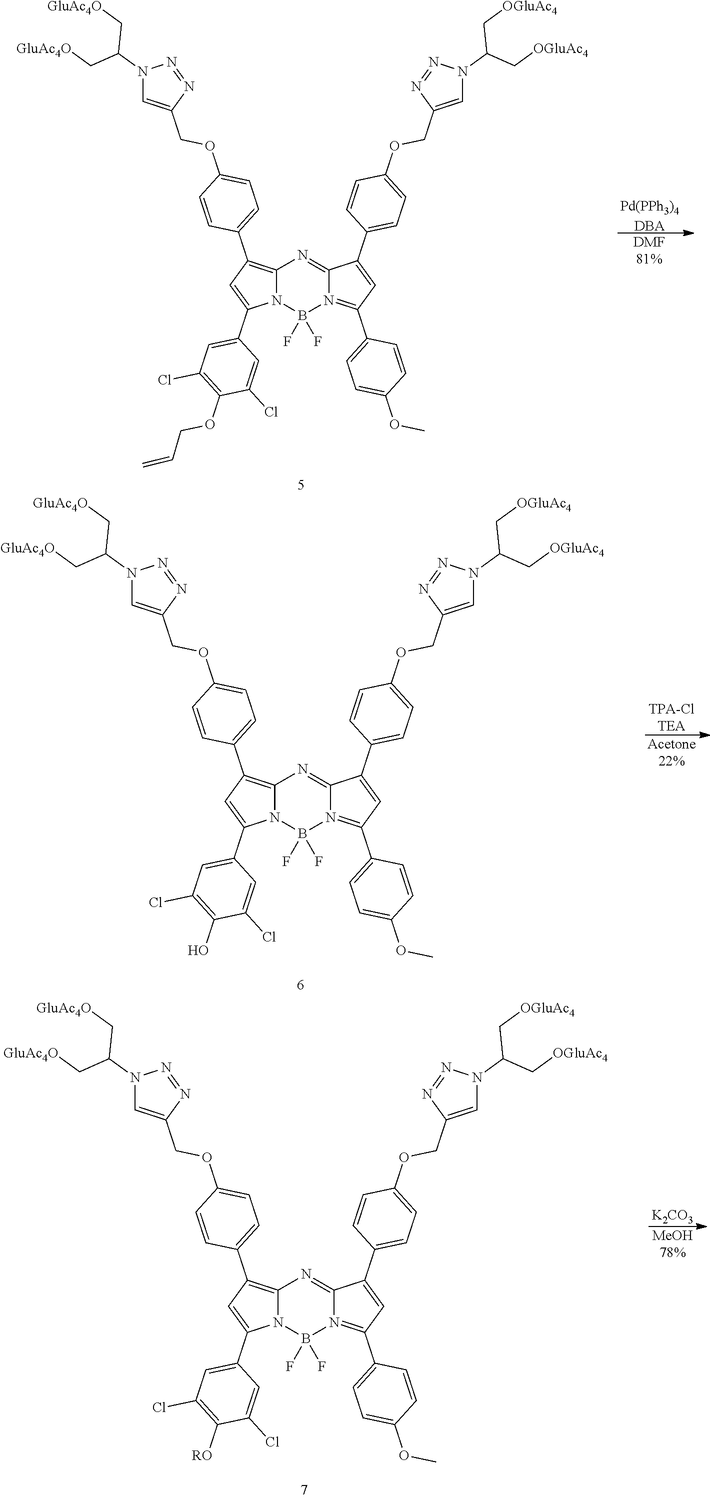

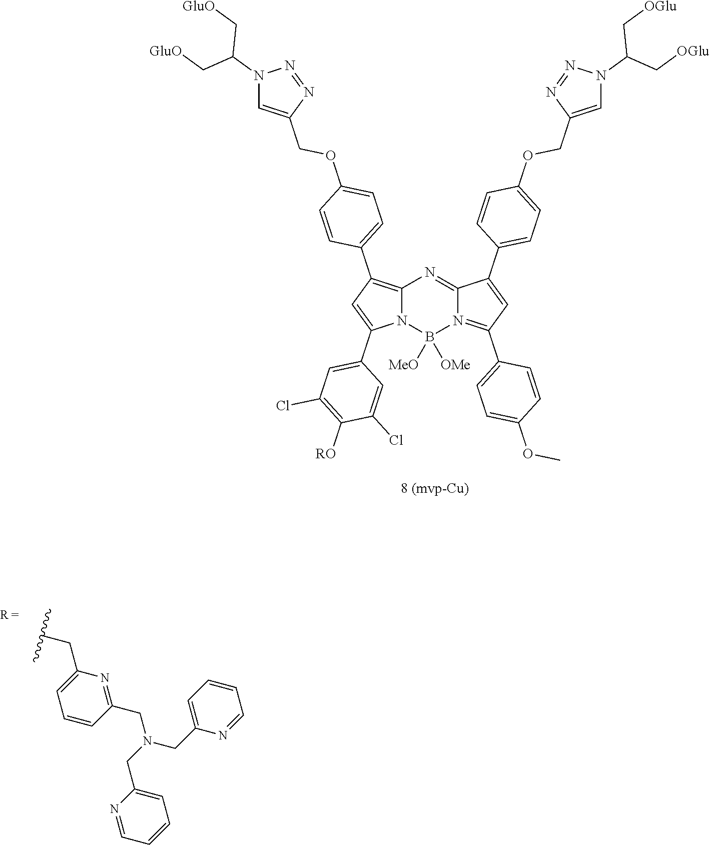

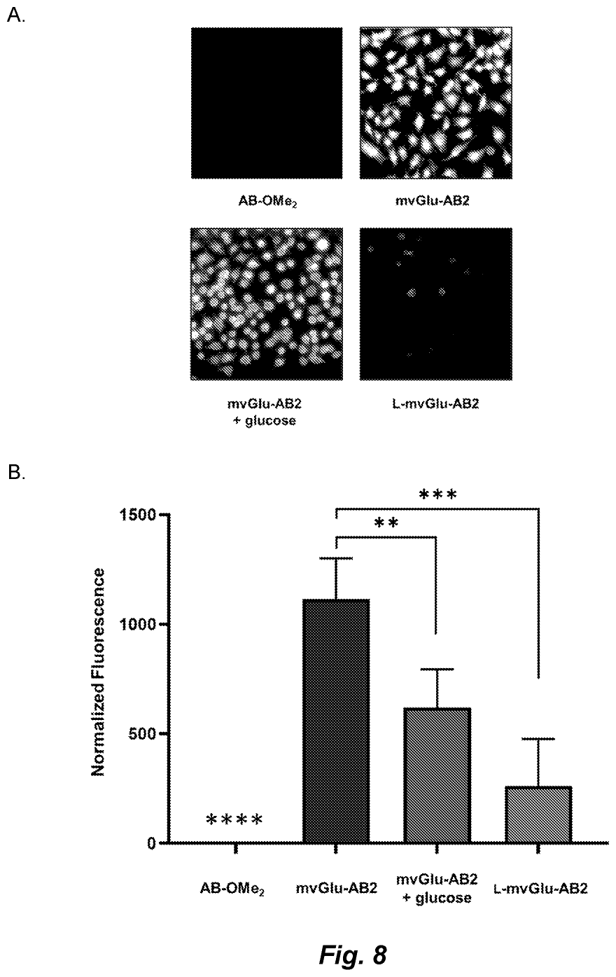

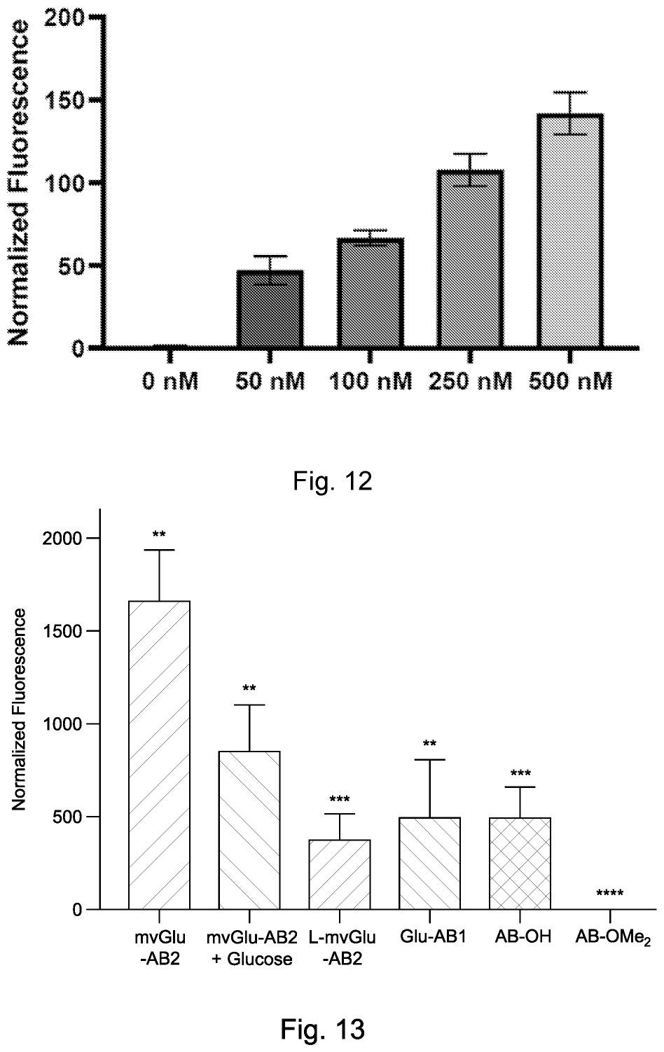

Evaluating the Cancer Targeting Ability of mvGlu-AB2 in Live Cells. Encouraged by these promising improvements, we advanced to cellular imaging to evaluate the targeting ability of mvGlu. For these experiments, we cultured A549 human lung cancer and 4T1 murine breast cancer cells because relative to their non-cancerous counterparts, GLUT1 is reported to be overexpressed by these solid tumors. First, A549 cells were stained with either AB-OMe₂ or mvGlu-AB2 and imaged after 20 minutes (FIG. 8a). The difference in signal intensity between the two conditions was dramatic, with the mvGlu-AB2-treated cells being >1000-fold brighter (FIG. 8b). Additionally, a clear fluorescent signature was apparent even at a low dye concentration of 50 nM, indicating excellent sensitivity (FIG. 12). Next, we performed two complimentary experiments to assess the involvement of GLUT1. First, A549 cells were pre-incubated with glucose (15 mM), before the addition of mvGlu-AB2 (2 μM) (FIG. 8a). The concentration of glucose was maintained throughout the duration of the experiment. We hypothesized if uptake of the dye involved GLUT1, the presence of excess glucose in the media would compete for uptake, resulting in a decrease of cellular fluorescence. This was confirmed as the signal intensity was 2-fold higher when glucose was absent (FIG. 8b).

To further corroborate these results, we synthesized 1-mvGlu-AB2 using 1-glucose instead of d-glucose. Because 1-glucose is not recognized by GLUT1, we anticipate that there will also be no interaction with the l-mvGlu isomer which will allow us to distinguish between nonspecific staining and GLUT1-mediated uptake. Analysis of the imaging results revealed cells treated with mvGlu prepared from the natural carbohydrate were 3-fold brighter (FIGS. 8a and 8b). This implicates the enhancement of water solubility is likely increasing the effective dye concentration in the media (c.f. results from AB-OMe₂ experiment) but ultimately, it is GLUT1 that is mediating significant dye uptake. Additional controls comparing cell permeability between ABs modified with AB-Glu1 and an oligoglycerol dendron are shown in FIG. 13. Moreover, similar results were obtained when these experiments were performed in 4T1 breast cancer cells (FIG. 13).

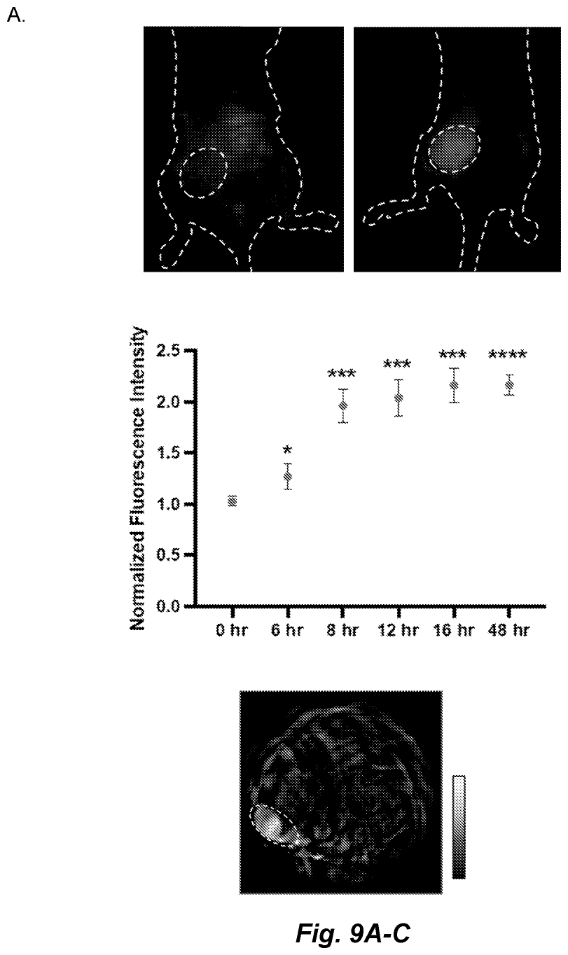

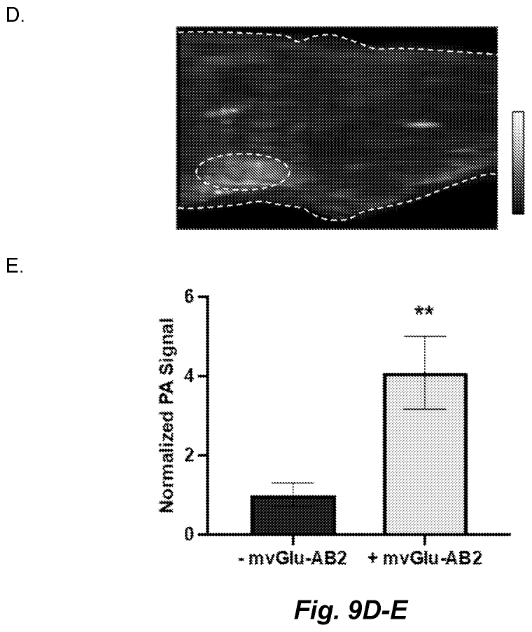

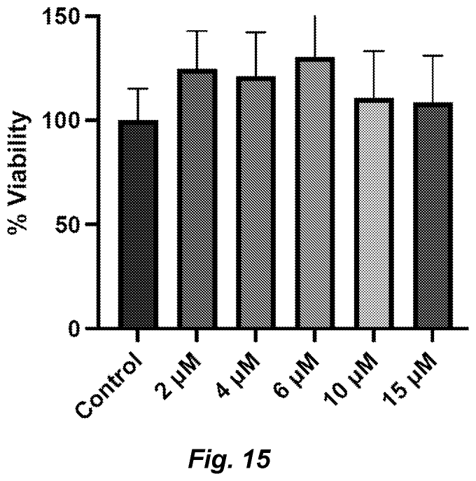

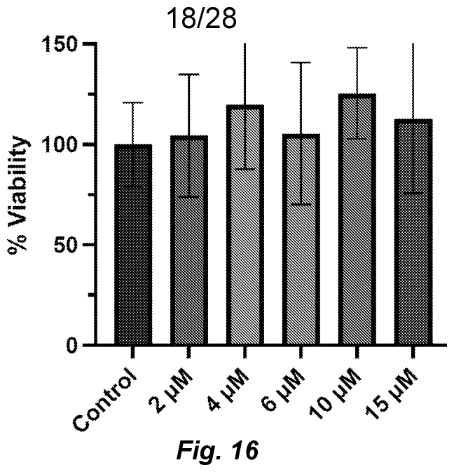

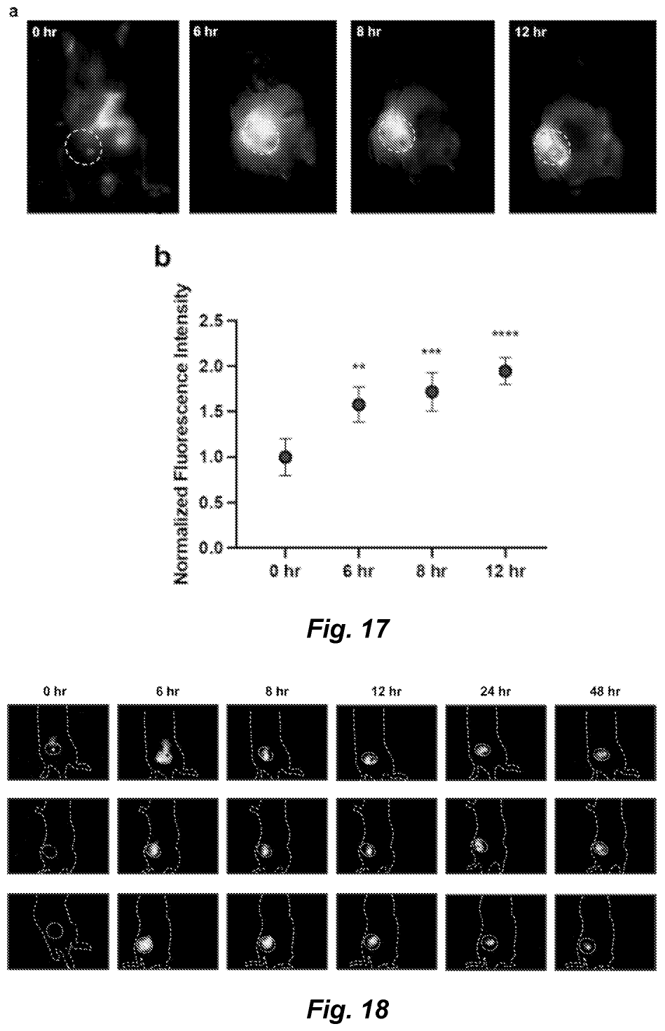

Application of mvGlu-AB2 to Image Tumors In Vivo. One of the most exciting envisioned applications of PA imaging agents augmented with robust tumor-targeting capabilities is diagnostic cancer imaging. For a dye to be suitable for this purpose it must be minimally cytotoxic. This property was assessed using a standard MTT assay where we found no loss in cell viability up to a dye concentration of 15 μM (6-hour incubation) (FIG. 15-16). In addition, after systemic administration, the dye must preferentially localize to the tumor site and the signal enhancement in the lesion should be at least 2-fold. A large dynamic range is important because it will decrease the likelihood of an incorrect assessment. To monitor biodistribution in orthotopic breast tumor-bearing BALB/c mice, we employed fluorescence imaging and found that relative to the initial background scan, the signal enhancement of the tumor site had reached ˜2-fold after 8 and 12 hours for mvGlu-AB2 (FIGS. 9a and 9b) and mvGlu-AB3 (FIG. 17), respectively. We corroborated these findings by performing PA imaging with spectral unmixing (FIG. 9c). Interestingly, the PA signal appears to span the entire tumor (FIG. 9d), and the difference between the tumor prior to and after injection of mvGlu-AB2 was substantial (4.1-fold) (FIG. 9e). The ability to specifically detect a tumor mass within deep tissue is a testament to the impressive PA properties of our new concentration of 50 nM, indicating excellent sensitivity (FIG. 12). Next, we performed two complimentary experiments to assess the involvement of GLUT1. First, A549 cells were pre-incubated with glucose (15 mM), before the addition of mvGlu-AB2 (2 μM) (FIG. 8a). The concentration of glucose was maintained throughout the duration of the experiment. We hypothesized if uptake of the dye involved GLUT1, the presence of excess glucose in the media would compete for uptake, resulting in a decrease of cellular fluorescence. This was confirmed as the signal intensity was 2-fold higher when glucose was absent (FIG. 8b).

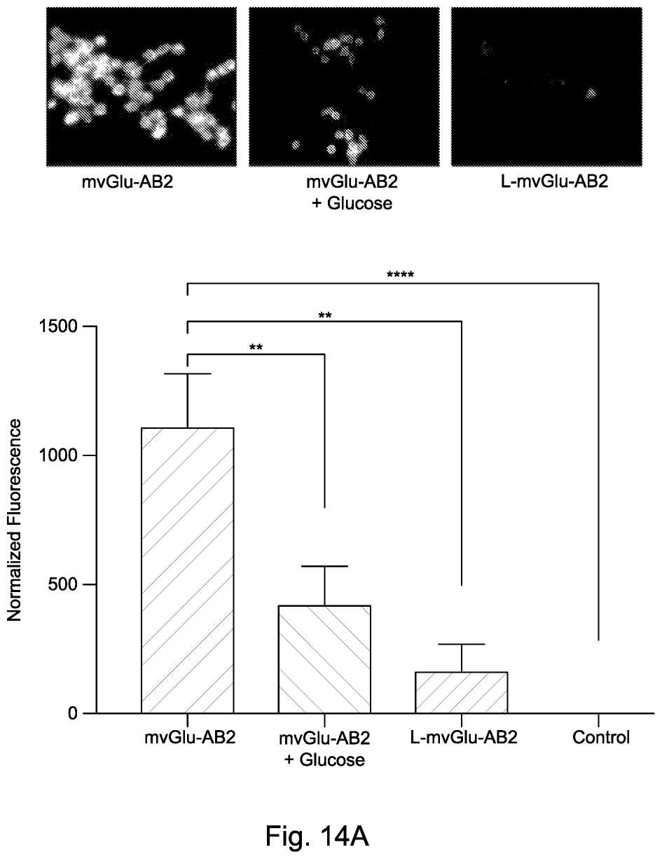

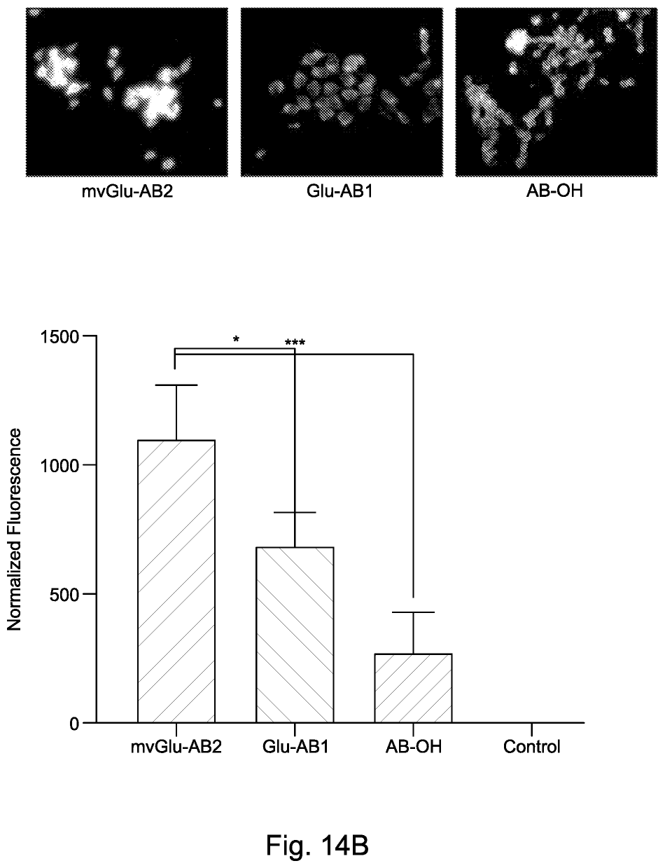

To further corroborate these results, we synthesized 1-mvGlu-AB2 using 1-glucose instead of d-glucose. Because l-glucose is not recognized by GLUT1, we anticipate that there will also be no interaction with the l-mvGlu isomer which will allow us to distinguish between nonspecific staining and GLUT1-mediated uptake. Analysis of the imaging results revealed cells treated with mvGlu prepared from the natural carbohydrate were 3-fold brighter (FIGS. 8a and 8b). This implicates the enhancement of water solubility is likely increasing the effective dye concentration in the media (c.f. results from AB-OMe₂ experiment) but ultimately, it is GLUT1 that is mediating significant dye uptake. Additional controls comparing cell permeability between ABs modified with AB-Glu1 and an oligoglycerol dendron are shown in FIG. 13. Moreover, similar results were obtained when these experiments were performed in 4T1 breast cancer cells (FIG. 14).

Application of mvGlu-AB2 to Image Tumors In Vivo. One of the most exciting envisioned applications of PA imaging agents augmented with robust tumor-targeting capabilities is diagnostic cancer imaging. For a dye to be suitable for this purpose it must be minimally cytotoxic. This property was assessed using a standard MTT assay where we found no loss in cell viability up to a dye concentration of 15 μM (6-hour incubation) (FIG. 15-16). In addition, after systemic administration, the dye must preferentially localize to the tumor site and the signal enhancement in the lesion should be at least 2-fold. A large dynamic range is important because it will decrease the likelihood of an incorrect assessment. To monitor biodistribution in orthotopic breast tumor-bearing BALB/c mice, we employed fluorescence imaging and found that relative to the initial background scan, the signal enhancement of the tumor site had reached ˜2-fold after 8 and 12 hours for mvGlu-AB2 (FIGS. 9a and 9b) and mvGlu-AB3 (FIG. 17), respectively. We corroborated these findings by performing PA imaging with spectral unmixing (FIG. 9c). Interestingly, the PA signal appears to span the entire tumor (FIG. 9d), and the difference between the tumor prior to and after injection of mvGlu-AB2 was substantial (4.1-fold) (FIG. 9e). The ability to specifically detect a tumor mass within deep tissue is a testament to the impressive PA properties of our new mvGlu-based dyes. Results obtained for mvGlu-AB3 were comparable (FIGS. 17-18).

Design, synthesis, and in vitro testing of mvpCu. In addition to developing powerful contrast agents, we wanted to extend the use of mvGlu to augment our analyte sensing capabilities. Recently, we developed an acoustogenic ABS probe for Cu(I) (named PACu-1), which we applied to visualize pathological levels of hepatic copper in a Wilson's disease model. PACu-1 is equipped with a tris[(2-pyridyl)methyl]amine (TPA) trigger that can be appended to an AB via an ether linkage. Upon coordination to Cu(I), an oxidative cleavage event can then take place which releases the dye. Of note, we selected the TPA trigger in this design because it has been used to develop a variety of sensors for other modalities and can effectively compete against glutathione to access intracellular Cu(I) (Scheme 2).

However, when PACu-1 was employed in an attempt to image breast tumors, most of the probe localized to the liver owing to its hydrophobic properties. This result was disappointing because elevated copper is linked to aggressive breast malignancies and its detection could serve as a diagnostic marker. Moreover, this finding highlights the unpredictable nature of many molecules not equipped with a targeting group. The design of the second-generation Cu probe to overcome this challenge is based on the mvGlu-AB3 architecture where one of the anisole rings was replaced with a 2,6-dichlorophenol moiety to provide a site for trigger installation. Synthesis of the proposed multivalent probe for Cu(I) (mvpCu), began with the preparation of 1 and 2, which were accessed from the corresponding chalcone precursors via aldol condensation and Henry reactions. The AB dye scaffold was synthesized via Paal-Knorr cyclization of the two precursors, followed by metalation using boron trifluoride etherate in 22% yield over 2-steps. Next, copper-mediated click chemistry with the acetyl protected mvGlu azide, yielded the glucose-incorporated AB 5. A Tsuji-Trost deallylation reaction was employed to remove the allyl protecting group on the 2,6-dichlorophenol moiety in 81% yield. The TPA trigger was then installed via nucleophilic substitution. Global deprotection of the acetyl groups with potassium methoxide generated in situ simultaneously exchanged the fluoro substituents with methoxy groups (Scheme 1).

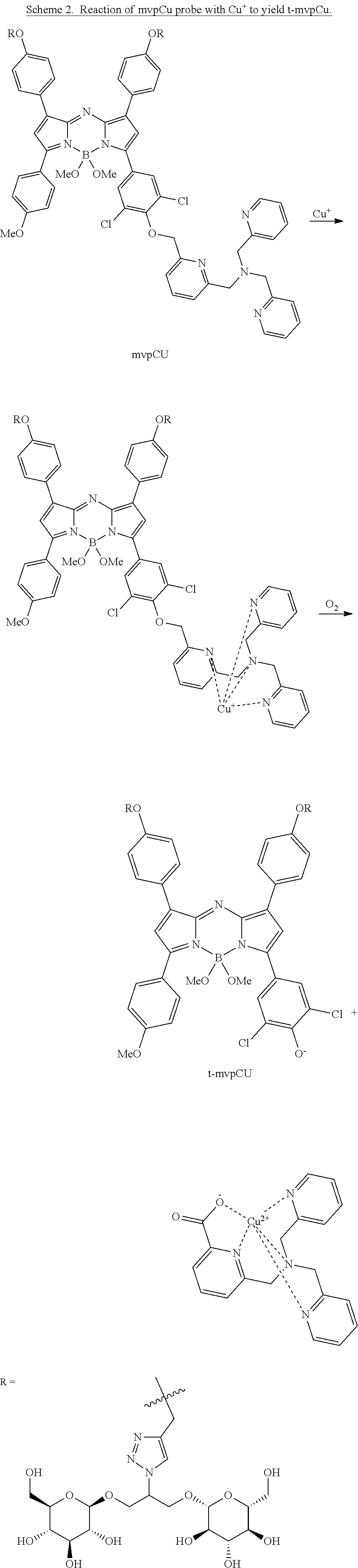

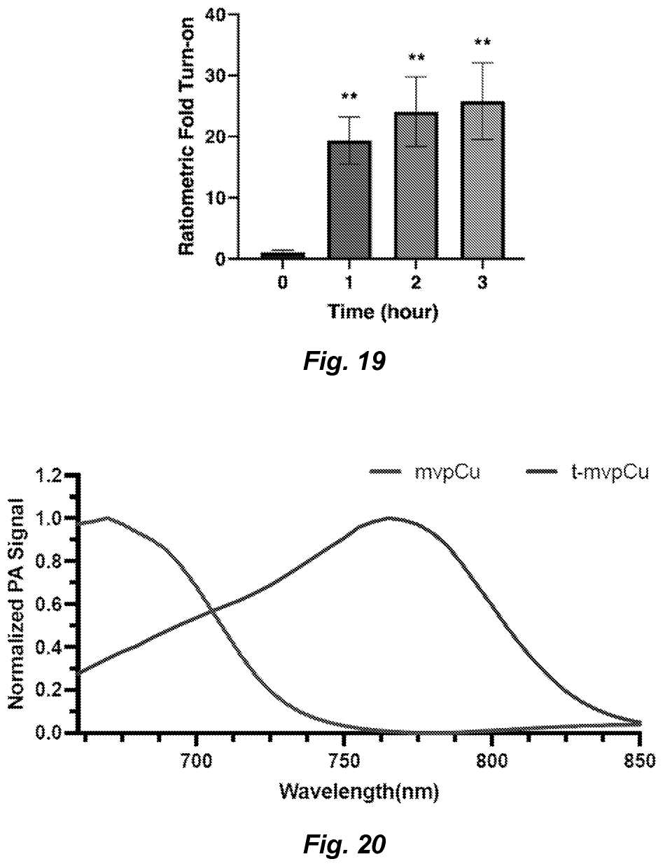

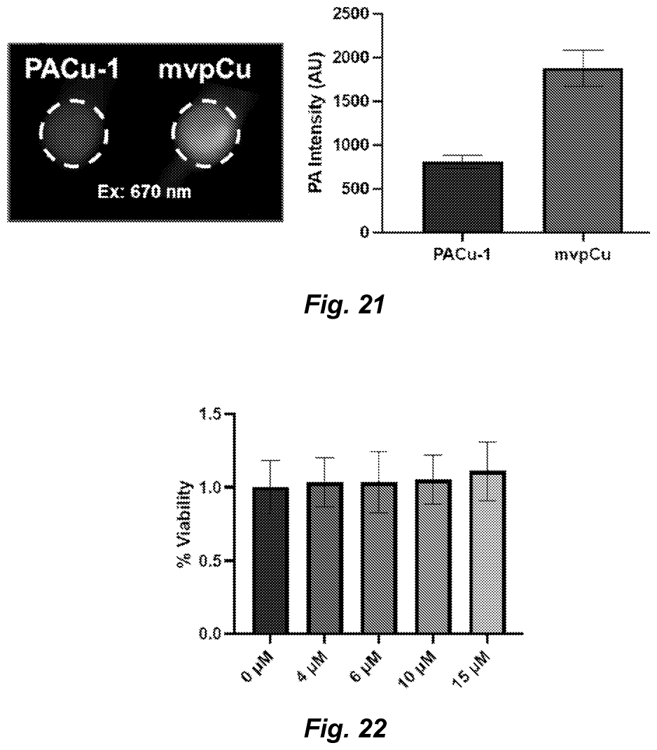

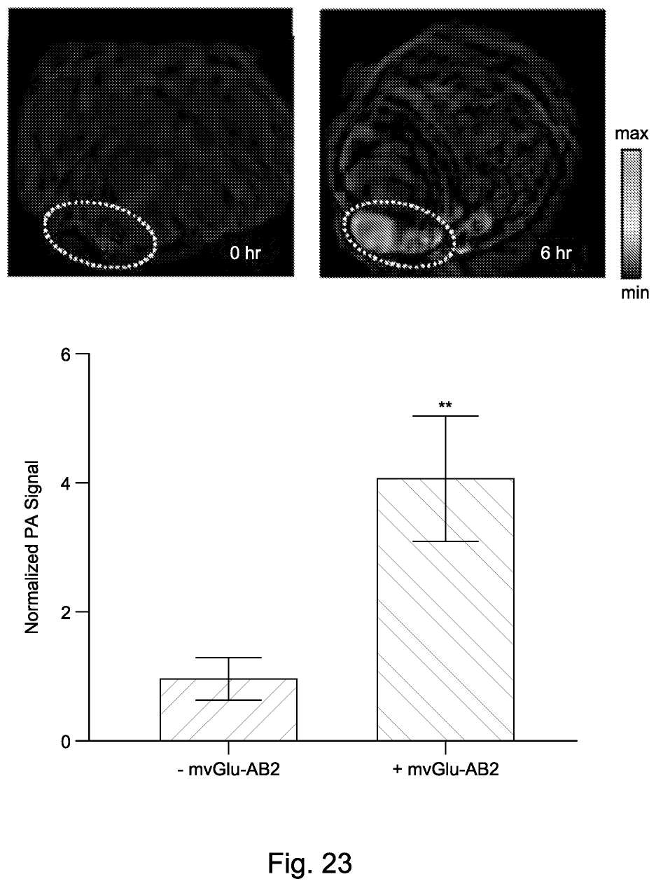

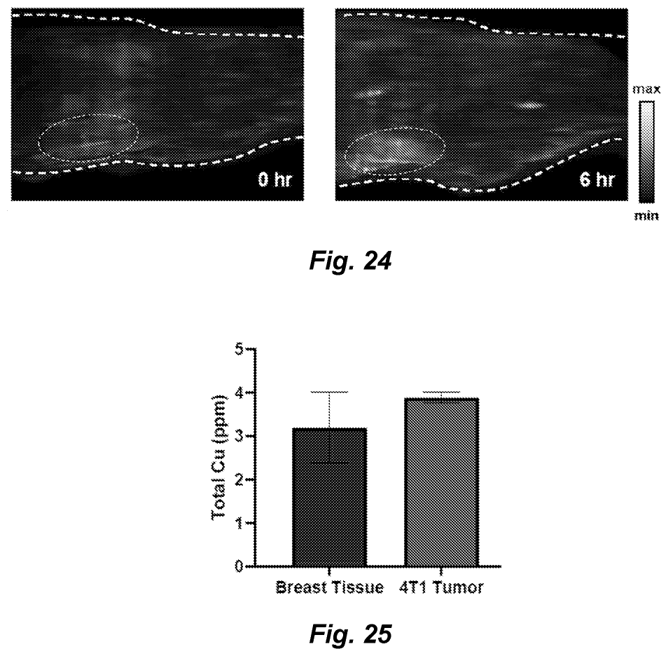

After synthesis, we obtained the absorbance spectra for mvpCu and the turned over product (t-mvpCu) (FIG. 10a). In both instances, the peaks were well defined which satisfied the criteria for in vivo spectral unmixing. Moreover, the spectra of mvpCu (λ_{abs}=676 nm) and t-mvpCu (λ_{abs}=754 nm) were sufficiently resolved, meaning we would be able to distinguish between the two species. Next, we tested the responsiveness of mvpCu to Cu(I) treatment. From a concentration of 0 to 100 μM, we observed a dose-dependent response (FIG. 10b). After 3 hours, probe activation was complete, and the corresponding turn-on response was 25.8±6.3-fold. Finally, we evaluated potential interference from other metal ions including alkali, alkaline earth, and transition metals. Under no circumstance did we observe interference (FIG. 10c). Finally, mvpCu and t-mvpCu (30 μM) were embedded in a tissue-mimicking phantom and imaged at the APA for the probe (670 nm) and turnover product (765 nm) using PA (FIG. 10d). Additional characterization, including MTT assays are shown in FIG. 19-22.

Application of mvpCu to detect intratumoral Cu (1) via PA imaging. The ability to employ mvpCu to image intracellular Cu(I) within tumors can be used for diagnostic purposes since high Cu(I) levels are linked to increased cancer aggression. To this end, 4T1 breast cancer cells were implanted in the mammary fat pads of BALB/c mice and once the tumor volume reached 100 mm³, we administered mvpCu systemically (1.0 mg/kg, 100 μL) for PA imaging. Because the turned over product is spectrally distinct from mvpCu, we were able to perform ratiometric imaging by irradiating each compound at 670 and 765 nm, respectively. We observed a 3.25-fold turn-on response relative to initial background scans, indicating successful detection of intratumoral Cu(I) (FIG. 11c). As mentioned above, because the TPA trigger used in this design is only able to detect the labile Cu pool (defined as Cu associated with glutathione), we also performed ICP-MS measurements to examine whether the total Cu content was also higher in the tumors. Although not statistically significant, our results appear to support this notion (breast tumors=3.88±0.12 ppm vs. healthy beast tissue=3.19±0.81 ppm) (FIG. 25). When the performance of PACu-1 was compared head-to-head to mvpCu, it failed to detect intratumoral Cu(I) (FIG. 11d). This finding demonstrates the powerful tumor targeting ability of our multivalent approach.

Conclusion. Cellular recognition is an important biological process, which allows cells to communicate, sense their environment, and in the case of immune cells, coordinate and mount an appropriate response. For instance, human cells feature a dense network of carbohydrates known as the glycocalyx. Depending on the type of saccharides present and their connectivity, these carbohydrates can serve as ligands for a specific binding partner (e.g., cell surface receptor). However, the binding affinity of a single carbohydrate residue is typically weak, but Nature has evolved to overcome this through a robust multivalent approach, where numerous copies of a carbohydrate are linked and presented in a manner to increase binding interactions. This incredibly simple solution to an otherwise challenging problem, inspired us to develop mvGlu, and in this study, we leveraged multivalent glucose targeting for delivery of NIR-I dyes.

In addition to being superior relative to the standard glucose ligand for targeting cancer, the mvGlu moiety is also a powerful aggregation blocking agent owing to its branched architecture which was enabled by the strategic use of glycerol as the scaffold. As we have demonstrated through various in vitro and in vivo spectral unmixing experiments, appending mvGlu to an AB result in a substantial gain in signal intensity. We anticipate mvGlu will be generalizable, where it will have a similar benefit when installed onto other imaging platforms (especially SWIR dyes). A boost in sensitivity is profound because this means a lower dye concentration will be needed to achieve the same readout and this results in less perturbation to the system under investigation.

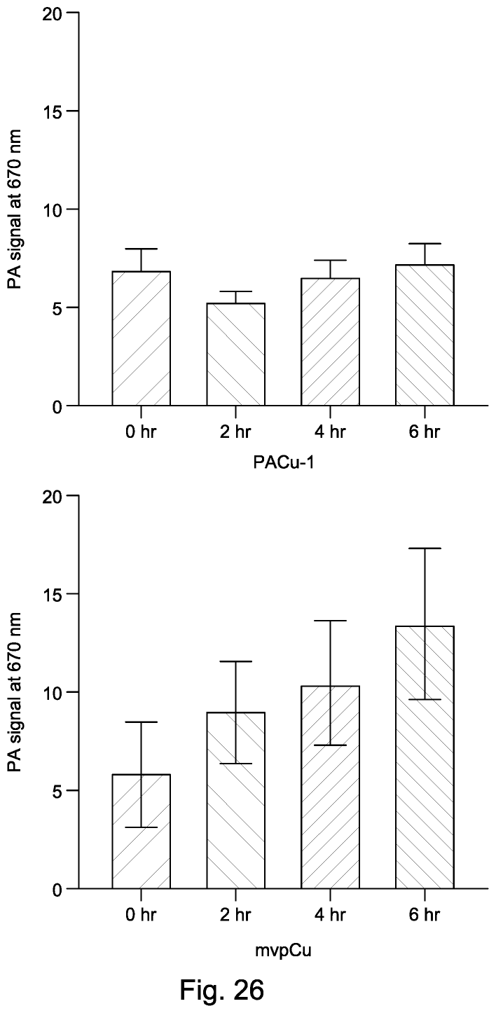

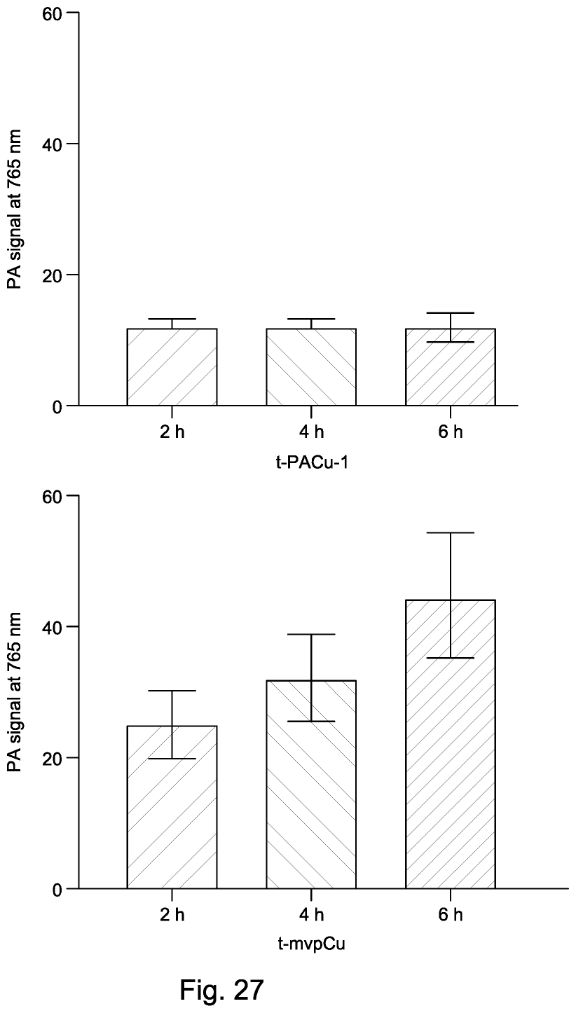

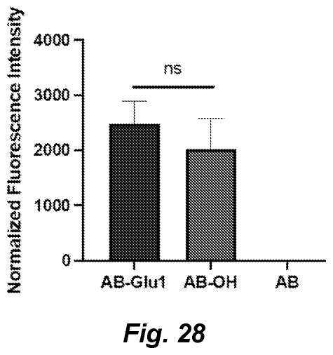

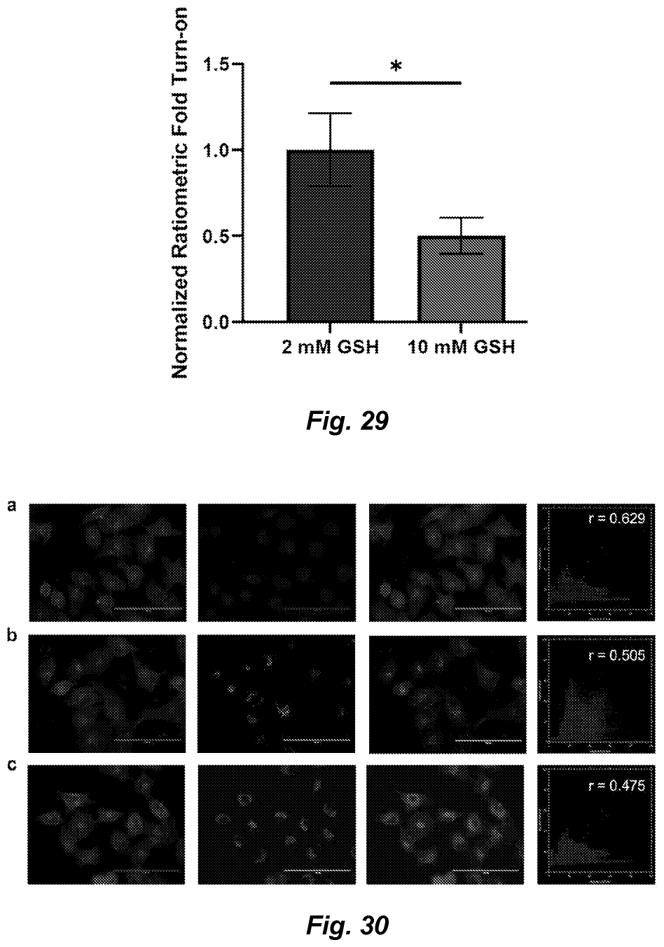

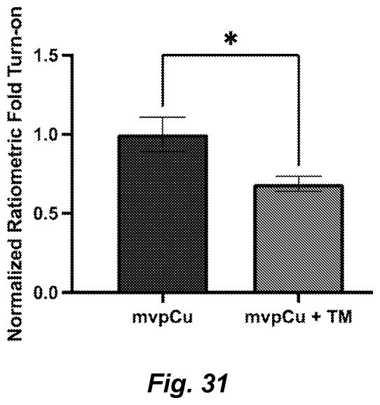

Lastly, the development of logic-gated ABS probes, where activation depends on the presence of two biomarkers (e.g., GLUT1 and Cu), hold tremendous promise for accurate and reliable assessment of a disease state. For instance, we have shown that mvpCu is able to effectively target cell populations overexpressing the GLUT1 protein, whereas PACu-1 cannot, by monitoring the PA signal of the tumor site upon irradiation with 670 nm light (FIG. 26). Moreover, because interaction with Cu(I) is required to red shift the APA via TPA trigger cleavage, observing the PA signal change at 765 nm allows us to confirm probe activation (FIG. 27). We would not expect to see significant change if the second biomarker was absent from the tumor site or if an imaging agent was not equipped with the Cu-responsive trigger (i.e., mvGlu-AB3).

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention provides therapeutic methods of treating cancer in a mammal, which involve administering to a mammal having cancer an effective amount of a compound or composition described herein. A mammal includes a primate, human, rodent, canine, feline, bovine, ovine, equine, swine, caprine, bovine and the like. Cancer refers to any various type of malignant neoplasm, for example, colon cancer, breast cancer, melanoma and leukemia, and in general is characterized by an undesirable cellular proliferation, e.g., unregulated growth, lack of differentiation, local tissue invasion, and metastasis.

The ability of a compound of the invention to treat cancer may be determined by using assays well known to the art. For example, the design of treatment protocols, toxicity evaluation, data analysis, quantification of tumor cell kill, and the biological significance of the use of transplantable tumor screens are known.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

Materials. Materials were purchased from commercial vendors and used without further purification. Thin layer chromatography (TLC) was performed on glass-backed TLC plates precoated with silica gel containing an UV254 fluorescent indicator (Macherey-Nagel). TLCs were visualized with a 254/365 nm UV hand-held lamp (UVP). Flash silica gel chromatography was performed using 0.04-0.063 mm 60 M silica (Macherey-Nagel). Deuterated solvents were purchased from Cambridge Isotope Laboratories. Thermo Fisher Scientific: n-BuOH, DCM, cupric sulfate, THF, DMF, acetone, acetonitrile, acetic anhydride, galacial acetic acid, sulfuric acid, ethyl acetate, molecular sieves 4 Å-8+12 (ca 2 mm) beads, ferric chloride, phosphate saline buffer (Corning), and toluene. The following chemicals were purchased from Oakwood Chemicals: FeSO₄·(H₂O)₇, potassium carbonate, sodium bicarbonate, sodium sulfate (anhydrous), ammonium acetate, sodium ascorbate, sodium thiosulfate anhydrous, potassium carbonate, triflic acid, propargyl bromide, p-hydroxybenzaldehyde, 2,6-dichlorophenol, nitromethane, 2,6-pyridinemethanol, 2-aminomethylpyridine, sodium borohydride, trichloroacetonitrile, 1,8-diazabicyclo[5.4.0]undec-7-ene (DBU), sodium azide, imidazole, 2-aminopropane-1,3-diol. The following chemicals were purchased from VWR: potassium hydroxide. The following chemicals were purchased from Sigma-Aldrich: tetrakis(acetonitrile)copper(I) hexafluorophosphate in acetonitrile, boron trifluoride etherate, palladium (II) tetrakis, acetyl chloride, thionyl chloride, sulfuryl chloride, and agarose. The following chemicals were purchased from AK Scientific: 1,3 dimethylbarbituric acid (DBA). Methanol and 28.0-30.0% ammonium hydroxide in water were purchased from Macron Fine Chemicals. The following chemicals were purchased from Alfa Aesar: triethylamine, allyl bromide, ethylenediamine. Iodine was purchased from TCI, and ethanol 200 proof was purchased from Decon Chemicals Inc. 1-glucose was purchased from Aaron Fine Chemicals. Lysotracker Green®, Mitotracker Green®, and DAPI were acquired from Thermo Fischer. DEA-NONOate was purchased from Cayman Chemicals. 2% milk was acquired from Starbucks.

Instruments and Software. ¹H and ¹³C NMR spectra were acquired on Varian 400, Varian 500, or Carver B500 spectrometers. The following abbreviations were used to describe coupling constants: singlet(s), doublet (d), triplet (t), quartet (q), quintet (quint), multiplet (m), and broad singlet (bs). Spectra were visualized and analysed using MestReNova (version 10.0). High-resolution mass spectra were acquired with a Waters Q-TOF Ultima ESI mass spectrometer. Fluorescence spectra were acquired on a QuantaMaster-400 scanning spectrofluorometer with micro fluorescence quartz cuvettes (Science Outlet). Cells were counted using the Countess II FL Cell Counter (Invitrogen, Thermo Fisher Scientific). All other data analysis was performed using Microsoft Excel or GraphPad Prism. Photoacoustic imaging was performed using the MSOT InVision 128 (iThera Medical). Reported values correspond to mean PA signals (Mean Pixel Intensity, MSOT a.u.) in regions of interest (ROIs) of equal area. Fluorescence imaging was performed using the CRi Maestro In-Vivo Fluorescence Imaging System. Images were analyzed using ImageJ and reported as the mean pixel intensity in ROIs of equal area.

Photophysical Characterization. Extinction coefficients and fluorescence quantum yields were acquired in experimental triplicates. Extinction coefficients were measured by performing serial dilutions of the compounds in 3:7 v/v MeCN:PBS (pH=7.4) within the linear range (absorbance values between 0.05-1.5). Reported fluorescence quantum yields are relative quantum yields compared to dimethoxy aza-BODIPY (Φ=0.36, chloroform). The compounds were titrated into acetonitrile or 3:7 MeCN/PBS (pH=7.4) such that their absorbance values were kept below 0.1 to prevent secondary absorbance events. The refractive index of 3:7 v/v MeCN:PBS (pH=7.4) was measured to be 1.343. Characterization of mvpCu and t-mvpCu were conducted in 1:1 v/v DMF:HEPES (50 mM, pH=7.4).

Log D_{7.4} Measurements. Log D measurements were conducted in technical triplicate and 5 experimental replicates. Samples at a known concentration (n=3) were added to Eppendorf tubes containing PBS (pH=7.4, 500 μL) and octanol (500 μL). The Eppendorf tubes were each vortexed for 30 seconds and subsequently subjected to microcentrifuge for 1 minute. Then the PBS and octanol layers were pipetted into a clear 96-well plate. The absorbance values were taken using a SpectraMax M2 plate reader. The log of (dye absorbance in octanol/dye absorbance in PBS) was calculated and averaged across different concentrations to generate the log D_{7.4}.

PA Imaging in Tissue-Mimicking Phantoms. Tissue phantoms were prepared by mixing agarose (750 mg) in milliQ water (49 mL). The solution was heated in a microwave oven in 30 second intervals until a viscous, homogeneous gel was formed. 2% milk (1 mL) was then added to the warm solution and mixed. The gel was transferred to 50 mL syringes with their tips removed. Plastic straws were put into the gel using a custom syringe holder. The phantom was allowed to harden for at least 30 minutes and then submerged in milliQ water within a 50 mL falcon tube. Sample solutions of the contrast agent (10 μM, 300 μL) were prepared in 3:7 v/v MeCN:PBS (pH=7.4), pipetted into plastic tubing, and sealed shut with hot glue. Sample solutions of mvpCu and t-mvpCu (10 μM) were prepared in 1:1 v/v DMF:(50 mM) Hepes, pH 7.4, pipetted into plastic tubing, and sealed shut with hot glue. PA measurements were taken at 5 nm intervals (660-1000 nm) and sample spectra were subtracted from background spectra of the buffer.

Cell Culture. A549 lung cancer cells and 4T1 murine breast cancer cells were acquired from ATCC. A549 cells were cultured in Ham's F-12K Medium containing 10% FBS and 1% penicillin streptomycin solution, and 4T1 cells were cultured in RPMI-1640 Medium containing 10% FBS and 1% penicillin streptomycin solution. Cells were incubated at 37° C. with 5% CO₂. Experiments were performed in 8-well plates (Nuc Lab-Tek Chambered Coverglass, Thermo Scientific) or 96-well plates (Nuclon Delta Surface Flat Bottom, Thermo Scientific).

MTT Cytotoxicity Assay. A 96-well plate was seeded with 30,000 cells per well and incubated at 37° C. with 5% CO₂ for 24-48 hours (˜70% confluent). The media was removed and replaced with fresh serum-free medium (200 μL) containing vehicle control (DMSO) or dye (4, 6, 10, or 15 μM). After 4 hours, the media was removed and replaced with a 20:1 mixture of PBS and (3-(4,5-dimethylthiazol-2-yl)-2,5-diphenyl-tetrazolium bromide (MTT, 5 mg/mL stock in PBS). The cells were incubated for 2 hours under the same conditions, and then the media was removed, replaced with DMSO (200 μL), and the absorbance of each well was recorded at 555 nm on a SpectraMax M2 plate reader. Percent viability was calculated relative to the vehicle control (n=8).

Cellular Uptake Studies. 8-well borosilicate plates were prepared with poly-L-Lysine coating and seeded with 50,000 cells per well and incubated at 37° C. with 5% CO₂ for 24 hours (˜60% confluent). Serum-free media containing mvGlu-AB2 (2 μM), 15 mM Glucose, mvGlu-AB2 (2 μM)+15 mM Glucose, L-mvGlu-AB2 (2 μM), dimethoxy aza-BODIPY (2 μM), or vehicle (DMSO) were prepared. Competition glucose wells were preincubated with 15 mM glucose in serum-free media for 30 minutes. Then the media was removed from each well, and each experimental condition was added and allowed to incubate for 30 minutes under the same conditions. The media was removed and replaced with PBS. Cells were imaged using an EVOS FL epifluorescence microscope with a Cy7 filter cube. Four experimental replicates and 3 technical replicates were taken.

Quantification of Cell Imaging Data. In FIJI (ImageJ), ROIs were drawn around representative cells and the integrated density (area×mean gray value) was measured. The total cell fluorescence was calculated by subtracting the integrated density of the cell by the background integrated fluorescence. Technical replicates (n=3) in each well were quantified as the average of 3 cells. Values are reported as the average (+SD) of 3 biological replicates (n=4). Colocalization Studies with mvGlu-AB2. 8-well borosilicate plates were prepared with poly-L-Lysine coating and seeded with 45,000 cells per well and incubated at 37° C. with 5% CO₂ for 24 hours (˜60% confluent). Serum-free media containing mvGlu-AB2 (1 μM) was prepared. Cells were incubated for 30 minutes with mvGlu-AB2. The cells were washed 1× with PBS and incubated with the relevant organelle tracker according to the manufacturer recommended protocol. Following incubation with tracker, the cells were washed 1× with PBS. Cells were imaged using an EVOS FL epifluorescence microscope. Lysotracker® Green and Mitotracker® Green were imaged using a GFP filter cube, DAPI was imaged using a DAPI filter cube, and mvGlu-AB2 was image with a Cy7 filter cube. Four experimental replicates and 3 technical replicates were taken.

In Vitro Characterization of mvpCu. The initial absorbance of mvpCu (5 μM) was taken in 1:1 v/v DMF:(50 mM) HEPES (pH=7.4, 2 mM GSH). Then, Cu(I) (100 μM) as tetrakis(acetonitrile)copper(I) hexafluorophosphate in acetonitrile was added to the solution. Absorbance readings were taken at timepoints at 1, 2, and 3 hours, where a maximum turn-on was seen at 3 hours. The ratiometric signal was calculated Final (Abs₇₅₀/Abs₆₈₀)/Initial (Abs₇₅₀/Abs₆₈₀) was calculated for each timepoint. Cu(I) turn-on was determined in experimental triplicate.

Stability Studies Against Metal Ions. The initial absorbance of dyes (5 μM, 1:1 v/v DMF:(50 mM) HEPES (pH 7.4, 2 mM GSH) was measured before addition of 100 μM of metal ions (unless otherwise noted). After addition, the samples were sealed and incubated at room temperature for 1 hour. Final absorbance measurements were recorded, and the relative stabiility was determined by calculating the ratiometric fold turn on for each metal species (final ratio of abs at 750/680 nm)/(initial ratio of abs at 750/680 nm). All metal ions were prepared from their chloride salts except for the following: Fe(II) was prepared from FeSO₄·(H₂O)₇ in water, Cu(I) was prepared from tetrakis(acetonitrile)copper(I) hexafluorophosphate in acetonitrile, and Ag(I) and Cs(I) were prepared from Ag₂CO₃ and Cs₂CO₃.

Assessment of Total Tumor Cu via ICP-MS. ICP-MS samples were prepared by sacrificing mice bearing tumors via isoflurane. The tumor and breast tissue were harvested from the mice, and the freshly harvested tissues were immediately submitted for ICP-MS analysis to the UIUC ICP-MS Analysis core facility. The PerkinElmer NexION 350D instrument was used to carry out measurements.

In Vivo Imaging. Before all imaging, hair on the bottom half of mice were removed via an electric razor and depilatory cream. In both FL and PA imaging, a background scan was taken of each mouse before injection. For PA imaging experiments, mice were allowed to equilibrate in the water tank for 10 min, and all scans were collected on H₂O mode at 34° C.

PA Imaging Unmixing Experiment in Tissue-Mimicking Phantoms. Tissue mimicking phantoms were prepared. mvGlu-AB2 or AB-OMe₂ (30 μM) were prepared in water containing hemoglobin from bovine blood (10 mg/mL). The solutions were pipetted in a plastic tube and sealed with hot glue. PA measurements were taken at 5 nm intervals (660-850 nm). The subsequent images were spectrally unmixed using the iThera Invision software with spectra of hemoglobin and the PA spectra of mvGlu-AB2 and AB-OMe₂. The resulting values at the λ_{PA} are reported as the mean PA signals (Mean Pixel Intensity, MSOT a.u).

Fluorescence Imaging with mvGlu-AB2 and mvGlu-AB3. 4T1 cells were trypsinized, neutralized with growth media, and counted on a Countess II FL Cell Counter (Invitrogen, Thermo Fisher Scientific). Cells were spun down, media was aspirated, and a solution was made in 1:1 serum-free RPMI media to VitroGel® hydrogel. Mice were injected in the mammary fat pad with 300,000 cells (50 μL) of the cell solution. Tumors grew for around 1.5-2 weeks and the mice were imaged once tumors reached 100 mm³. Dyes (1.2 mg/kg) were dissolved in 0.9% saline (5% DMSO) and injected retroorbitally. Mice were imaged using the CRi Maestro In-Vivo Fluorescence Imaging System at timepoints=0, 6, 8, 12, 16, 24, and 48 hours. Excitation occurred at 680 nm, and light was collected from 690-850 nm (n=3).

PA Imaging with mvGlu-AB2. 4T1 cells were trypsinized, neutralized with growth media, and counted on a Countess II FL Cell Counter (Invitrogen, Thermo Fisher Scientific). Cells were spun down, media was aspirated, and a solution was made in 1:1 serum-free RPMI media to Geltrex™ LDEV-Free, hESC-Qualified, Reduced Growth Factor Basement Membrane Matrix. Mice were injected in the mammary fat pad with 250,000 cells (50 μL) of the cell solution. Tumors grew for around 1.5-2 weeks and the mice were imaged once tumors reached 100 mm³. mvGluAB-2 was reconstituted in 0.9% saline (5% DMSO) and injected retroorbitally. PA images were taken at timepoints=0 and 6 hours in 5 nm increments from 660-700 nm. Images were spectrally unmixed from hemoglobin, deoxyhemoglobin, and melanin (n=4).

PA Imaging with mvpCu and PACu-1. 4T1 cells were trypsinized, neutralized with growth media, and counted on the Countess II FL Cell Counter (Invitrogen, Thermo Fisher Scientific). Cells were spun down, media was aspirated, and a solution was made in 1:1 serum-free RPMI media to Geltrex™ LDEV-Free, hESC-Qualified, Reduced Growth Factor Basement Membrane Matrix. Mice were injected in the mammary fat pad with 500,000 cells (50 μL) of the cell solution. Tumors grew for around 1.5-2 weeks and the mice were imaged once tumors reached 100 mm³. Dyes was reconstituted in 0.9% saline (5% DMSO) and injected retroorbitally (1.0 mg/kg). PA images were taken at timepoints=0, 2, 4, and 6 hours at 660, 665, 670, 675, 680, 745, 750, 755, 760, and 765 nm. Images were spectrally unmixed from hemoglobin, deoxyhemoglobin, and melanin (n=4).

Liquid Chromatography Data. LC-HRMS analyses of reported compounds are discussed below. Products were separated on a CORTECS™ UPLC C18 column (1.6 μm, 2.1 by 50 mm) with a linear gradient using a combination of solvent A (95% water, 5% acetonitrile, 0.1% formic acid) and solvent B (95% acetonitrile, 5% water, 0.1% Formic acid) at a flow rate of 0.4 mL/minute. Linear gradient protocol in minutes: 0 (90% A), 0.5 (90% A), 3 (90% A), 5 (40% A), 6.5 (10% A), 8 (0% A), 8.1 (90% A), 10 (90% A). Time of gradient was 10 minutes.
Integrated LC TIC trace of mvpCu. Retention time (relative scale): 0.38; 97% AUC.
Integrated LC TIC trace of mvGlu-AB2. Retention time (relative scale): 0.40; 100% AUC.
Integrated LC TIC trace of mvGlu-AB3. Retention time (relative scale): 0.41; 100% AUC.
Integrated LC TIC trace of AB-Glu1. Retention time (relative scale): 0.40; 100% AUC.
Integrated LC TIC trace of AB-OH. Retention time (relative scale): 0.40; 100% AUC.

See J. Am. Chem. Soc. 2023, 145, 7313-7322, Supplementary Information (pubs.acs.org/doi/10.1021/jacs.2c13489), for chromatograms and additional information. See also, Potter et al. J. Org. Chem. 2016, 81, 8, 3443-3446, doi.org/10.1021/acs.joc.6b00177 and Reinhardt et al. J. Am. Chem. Soc. 2018, 140, 3, 1011-1018. doi.org/10.1021/jacs. 7b10783.

### Example 2. Synthetic Procedures

3-(4-(Allyloxy)-3,5-dichlorophenyl)-5,5-difluoro-7-(4-methoxyphenyl)-1,9-bis(4-(prop-2-yn-1-yloxy)phenyl)-5H-514.614-dipyrrolo[1,2-c:2′,1′-f][1,3,5,2]triazaborinine (4). To a round-bottom flask, 1 (693 mg, 1.5 mmol, 1 equiv.) and 2 (1.1 g, 3.1 mmol, 2 equiv.) were suspended in n-butanol (9.3 mL). The solution was heated to 130° C. for 45 minutes in an oil bath containing rice bran oil. Then, ammonium acetate (1.8 g, 23 mmol, 15 equiv.) was added, and the reaction was allowed to heat at 130° C. overnight. The reaction was cooled to room temperature, diluted with water, and extracted with DCM (3×). The organic layers were combined, dried with sodium sulfate, and concentrated via rotary evaporation. The excess n-butanol was removed azeotropically with toluene. The resulting shiny blue solid was used without further purification. The solid was dissolved in DCM (150 mL) and cooled to 0° C. DIPEA (2.7 mL, 15 mmol, 10 equiv.) and BF₃·OEt₂ (2.7 mL, 22 mmol, 14 equiv.) were added sequentially. The reaction was warmed to room temperature and was allowed to stir overnight. The reaction was quenched with aqueous sodium bicarbonate and extracted with DCM (3×). The combined organic layers were dried with sodium sulfate and concentrated via rotary evaporation. The resulting solid was purified via column chromatography (SiO₂, toluene) to afford the desired compound as a blue solid (0.26 mg, 0.34 mmol, 22%). ¹H NMR (500 MHz, CDCl₃) δ 8.07 (d, J=8.9 Hz, 2H), 7.99 (d, J=8.8 Hz, 2H), 7.95 (d, J=8.9 Hz, 2H), 7.88 (s, 2H), 7.04-6.94 (m, 6H), 6.74 (s, 1H), 6.11 (ddt, J=16.5, 10.4, 5.9 Hz, 1H), 5.41 (dd, J=17.2, 1.6 Hz, 1H), 5.25 (dd, J=10.3, 1.4 Hz, 1H), 4.72 (dd, J=5.0, 2.4 Hz, 4H), 4.57 (dd, J=6.0, 1.3 Hz, 2H), 3.84 (s, 3H), 2.53 (dt, J=3.8, 2.4 Hz, 2H). ¹³C NMR (125 MHz, CDCl₃) δ 163.0, 159.3, 158.7, 133.1, 132.3, 131.2, 130.8, 129.8, 125.7, 123.5, 119.0, 115.3, 115.2, 114.7, 78.4, 76.2, 76.1, 74.7, 56.0, 55.7. ESI HR-MS: Calculated for C₄₂H₃₀BCl₂N₃O₄ [M]− m/z 759.1674, found 759.1675.

Compound 5. To a round bottom flask, 4 (489 mg, 0.64 mmol, 1 equiv.), 20 (1.5 g, 1.9 mmol, 3 equiv.), and CuSO₄·H₂O (1.6 g, 6.4 mmol, 10 equiv.) were dissolved in THF (32 mL). Sodium ascorbate (650 mg, 3.3 mmol, 5.1 equiv.) was dissolved in water (6.5 mL) and was added to the reaction suspension dropwise. The reaction was allowed to stir at room temperature overnight. The reaction was diluted with water and extracted with EtOAc (3×). The combined organic layers were dried with sodium sulfate and concentrated via rotary evaporation. The resulting blue oil was purified via column chromatography (SiO₂, 4:1 v/v EtOAc:Hexane then 1:19 v/v MeOH:DCM) to afford the desired compound as a blue solid (1.0 g, 0.44 mmol, 68%). ¹H NMR (500 MHz, DMSO-d₆) δ 8.29-8.20 (m, 2H), 8.19-8.13 (m, 2H), 8.11-8.04 (m, 1H), 8.00 (s, 2H), 7.33-7.23 (m, 4H), 7.18 (d, J 8.6 Hz, 1H), 6.97 (d, J=8.3 Hz, 2H), 6.10 (ddt, J=16.4, 11.8, 5.0 Hz, 1H), 5.59 (dtd, J=22.4, 17.2, 5.3 Hz, 2H), 5.51-5.38 (m, 2H), 5.35-5.17 (m, 5H), 5.11-5.05 (m, 2H), 4.99 (dt, J=6.3, 3.5 Hz, 2H), 4.91 (dt, J=9.2, 2.6 Hz, 3H), 4.86-4.61 (m, 10H), 4.23-4.07 (m, 5H), 4.02-3.96 (m, 8H), 3.93 (s, 3H), 3.86 (dt, J=9.5, 4.6 Hz, 4H), 2.31-1.65 (m, 48H). ¹³C NMR (125 MHz, CDCl₃) δ 170.6, 170.0, 169.5, 169.4, 169.2, 157.8, 155.3, 133.1, 132.9, 132.3, 132.1, 131.3, 131.0, 130.6, 130.3, 129.6, 129.5, 128.7, 128.6, 121.1, 119.4, 118.8, 115.3, 114.4, 100.9, 100.8, 100.5, 96.9, 79.5, 74.6, 74.5, 73.2, 72.4, 72.3, 72.0, 71.9, 70.9, 69.9, 68.2, 68.1, 67.9, 67.2, 62.9, 61.7, 61.6, 55.5, 41.4, 38.4, 20.7, 20.7, 20.6, 20.5, 20.5.

Compound 6. To a round bottom flask, 5 (324 mg, 0.14 mmol, 1 equiv.) and 1,3-dimethylbarbituric acid (35.0 mg, 0.22 mmol, 1.6 equiv.) were dissolved in DMF (16 mL). The reaction was degassed by bubbling N₂ for 5 minutes. Then, palladium tetrakis (48.5 mg, 0.042 mmol, 0.3 equiv.) was added, and the reaction stirred at room temperature for 2 hours. The solvent was concentrated via rotary evaporation and directly purified via column chromatography (SiO₂, 4 1:99 v/v MeOH:DCM then 1:19 v/v MeOH:DCM with 1% AcOH) to afford the desired compound as a blue-purple solid (0.26 mg, 0.11 mmol, 81%). ¹H NMR (500 MHz, DMSO-d₆) δ 8.43-8.03 (m, 6H), 7.65-7.59 (m, 6H), 7.57-7.47 (m, 3H), 7.26 (d, J=8.3 Hz, 3H), 7.05 (t, J=8.6 Hz, 1H), 5.57 (ddt, J=18.8, 9.4, 7.4 Hz, 4H), 5.29-5.17 (m, 5H), 4.90 (dd, J=9.9, 4.4 Hz, 4H), 4.80-4.66 (m, 4H), 4.16 (s, 5H), 4.09 (s, 5H), 4.00 (m, 8H), 3.90 (s, 3H) 3.86 (m, 4H), 2.10-1.84 (m, 48H). ¹³C NMR (125 MHz, CD₃OD) δ 170.9, 170.2, 170.0, 169.8, 169.5, 150.9, 131.8, 131.7, 130.8, 128.6, 128.5, 121.1, 119.7, 114.0, 100.7, 96.9, 72.4, 71.6, 71.0, 69.9, 68.2, 63.0, 61.6, 56.9, 55.0, 42.8, 29.4, 27.5, 19.9, 19.8, 19.6.

Compound 7. To a round bottom flask, TPA-Cl (32.8 mg, 0.097 mmol, 1.1 equiv.) and sodium iodide (73.0 mg, 0.44 mmol, 5 equiv.) were dissolved in acetone (8.8 mL) and heated to reflux for 1 hour. The reaction was cooled. Then, 6 (200.2 mg, 0.088 mmol, 1 equiv.) and triethylamine (0.037 mL, 0.27 mmol, 3 equiv.) were added, and the reaction was heated to 40° C. overnight. The reaction was cooled, diluted with brine, and extracted with DCM (3×). The combined organic layers were dried with sodium sulfate and concentrated via rotary evaporation. The crude residue was purified via flash chromatography on neutral alumina (Al₂O₃, DCM then 1:19 v/v MeOH:DCM) to afford the product as a blue-green solid (49.9 mg, 0.019 mmol, 22%). ¹H NMR (500 MHz, 1:1 v/v CD₂Cl₂/CD₃OD) δ 8.33 (d, J=5.1 Hz, 2H), 7.84 (d, J=8.5 Hz, 2H), 7.68 (dt, J=31.4, 7.7 Hz, 4H), 7.53 (dt, J=12.8, 7.0 Hz, 16H), 7.46-7.38 (m, 10H), 7.24 (s, 3H), 7.21-7.09 (m, 2H), 6.89 (d, J=8.1 Hz, 2H), 5.30-5.23 (m, 2H), 5.18-5.08 (m, 4H), 5.05 (s, 2H), 4.96 (t, J=9.8 Hz, 2H), 4.90-4.69 (m, 3H), 4.64-4.40 (m, 3H), 4.16 (td, J=12.4, 7.0 Hz, 4H), 4.08-4.02 (m, 6H), 3.75 (d, J=7.1 Hz, 3H), 3.71 (d, J=10.1 Hz, 3H), 2.13-1.77 (m, 48H). ¹³C NMR (125 MHz, 1:1 v/v CD₂Cl₂/CD₃OD) δ 194.4, 171.1, 171.0, 170.9, 170.5, 170.5, 170.3, 170.0, 170.0, 169.8, 169.7, 169.6, 169.5, 163.2, 158.9, 158.7, 157.8, 155.1, 154.9, 148.3, 143.3, 137.6, 137.2, 133.7, 133.6, 133.5, 132.7, 132.7, 132.4, 132.3, 131.8, 131.8, 131.7, 130.9, 129.9, 128.8, 128.8, 128.7, 128.7, 128.6, 128.1, 123.6, 123.5, 123.3, 123.2, 122.4, 121.1, 120.6, 115.0, 100.7, 100.4, 89.7, 79.6, 75.3, 72.9, 72.7, 72.5, 72.4, 71.8, 71.7, 71.5, 71.4, 71.0, 70.9, 70.1, 69.7, 68.8, 68.4, 68.2, 68.2, 67.9, 67.0, 66.6, 63.1, 62.2, 61.7, 61.6, 61.5, 60.5, 59.9, 59.8, 41.5, 38.5, 36.2, 35.4, 30.9, 30.1, 29.5, 27.7, 27.1, 27.0, 22.5, 20.2, 20.1, 20.1, 20.0, 20.0, 20.0, 20.0.

mvpCu (8). To a round bottom flask, 7 (42.2 mg, 0.016 mmol, 1 equiv.) and potassium carbonate (42.5 mg, 0.31 mmol, 18.8 equiv.) were suspended in methanol (1.6 mL). The reaction was stirred at room temperature for 3 hours. Then, the solvent was removed via rotary evaporation and purified via reverse-phase chromatography (C18, 100% H2O→50% Acetonitrile/H2O) to afford the product as a blue solid (24.6 mg, 0.013 mmol, 78%). ¹H NMR (500 MHz, 1:1 v/v CD₃CN:D₂O) 8.45 (s, 4H), 8.38-7.98 (m, 3H), 7.93 (s, 2H), 7.81 (d, J=25.7 Hz, 3H), 7.70-7.53 (m, 5H), 7.30 (d, J 14.0 Hz, 5H), 7.01 (s, 3H), 6.35 (s, 1H), 5.23 (s, 8H), 4.40 (s, 7H), 4.17 (d, J=30.7 Hz, 2H), 4.02 (s, 3H), 3.89 (s, 5H), 3.83 (s, 6H), 3.80 (s, 2H), 3.71 (s, 10H), 3.58 (s, 4H), 3.53-3.15 (m, 6H). ¹³C NMR (125 MHz, 1:1 v/v CD₃CN:D₂O) δ 165.5, 163.2, 162.9, 162.7, 162.1, 159.2, 159.2, 159.2, 158.9, 157.3, 155.6, 149.0, 148.3, 137.9, 133.8, 132.6, 132.5, 132.3, 130.4, 129.9, 129.9, 129.8, 129.0, 125.2, 123.7, 116.5, 116.4, 115.5, 103.9, 103.9, 103.3, 103.2, 98.0, 76.9, 76.8, 76.5, 76.1, 76.0, 73.8, 70.4, 69.0, 68.1, 62.1, 61.9, 61.8, 61.8, 61.8, 61.7, 60.9, 60.8, 60.7, 60.2, 53.9, 23.7.

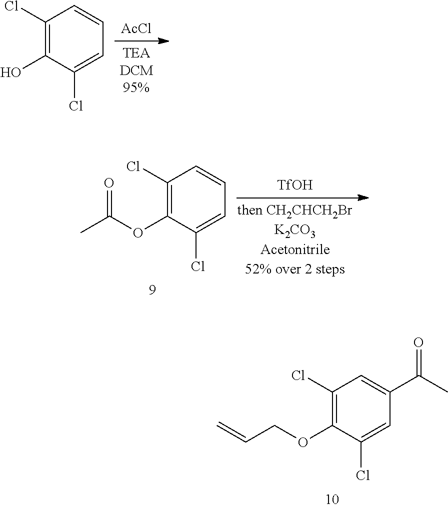

2,6-Dichlorophenyl acetate (9). 2,6-Dichlorophenol (50.0 g, 307 mmol, 1 equiv.) and triethylamine (107 mL, 767 mmol, 2.5 equiv.) were dissolved in DCM (550 mL). Then, the reaction was cooled to 0° C., and acetyl chloride (26 mL, 368 mmol, 1.2 equiv.) was added dropwise. The reaction was warmed to room temperature and was allowed to stir for 4 hours. The reaction was quenched with aqueous sodium bicarbonate until a pH of 8 was achieved and then extracted with DCM (3×). The combined organic layers were dried with sodium sulfate and concentrated via rotary evaporation. The oily residue was purified via column chromatography (SiO₂. 1:9 v/v EtOAc:Hexanes) to afford the desired product as a light yellow oil (60.1 g, 293 mol, 95%). ¹H NMR (500 MHz, CDCl₃) δ 7.36 (d, J=8.1 Hz, 2H), 7.14 (t, J=8.1 Hz, 1H), 2.40 (s, 3H). ¹³C NMR (125 MHz, CDCl₃) δ 167.3, 144.1, 128.9, 128.6, 128.3, 127.2, 121.2, 20.2. ESI HR-MS: Calculated for C₁₁H₁₁O₂Cl₂ [M-H]⁺ m/z 245.0136, found 245.0134.

1-(4-(Allyloxy)-3,5-dichlorophenyl) ethan-1-one (10). To a round-bottom flask, 9 (66.9 g, 326 mmol, 1 equiv.) was dissolved in TfOH (125 mL). The solution was heated to 40° C. overnight. The reaction was cooled to 0° C. and quenched with aqueous sodium bicarbonate until a pH of 7 was reached. The aqueous solution was extracted with EtOAc (3×). The combined organic layers were dried with sodium sulfate and concentrated via rotary evaporation. The resulting white solid was used without further purification. The crude intermediate and potassium carbonate (83.6 g, 605 mmol, 2 equiv.) were suspended in acetonitrile (302 mL) and allyl bromide (42 mL, 484 mmol, 1.6 equiv.) was added. The reaction was heated to reflux and stirred for 1 hour. Following completion, the reaction was filtered via vacuum filtration and the filtrate was concentrated via rotary evaporation. The resulting residue was purified via column chromatography (SiO₂, 1:9 v/v EtOAc:Hexanes) to afford the product as a yellow oil (39 g, 160 mmol, 52%). ¹H NMR (400 MHz, CDCl₃) δ 7.88 (s, 2H), 6.14 (ddt, J=16.6, 11.6, 6.0 Hz, 1H), 5.48-5.39 (m, 1H), 5.30 (d, J=10.3 Hz, 1H), 4.64 (d, J=6.0 Hz, 2H), 2.56 (s, 3H). ¹³C NMR (125 MHz, CDCl₃) δ 194.8, 155.1, 133.9, 132.5, 130.2, 129.1, 119.4, 74.6, 26.4.

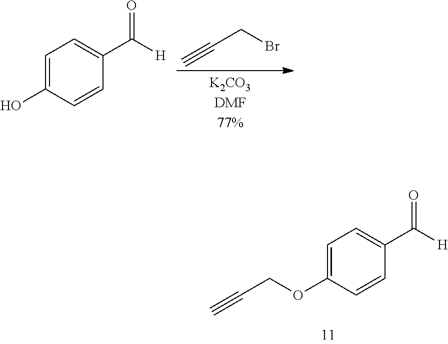

4-(Prop-2-yn-1-yloxy)benzaldehyde (11). 4-Hydroxybenzaldehyde (40 g, 330 mmol, 1 equiv.) was dissolved in DMF (410 mL). Potassium carbonate (59 g, 430 mmol, 1.3 equiv.) and propargyl bromide (32 mL, 430 mmol, 1.3 equiv.) were added sequentially. The reaction was heated to 60° C. for 8 hours. The reaction was cooled to room temperature and diluted with water. Clear, sharp crystals began to precipitate from the solution, and the reaction mixture was allowed to stand overnight. The resulting precipitate was isolated via vacuum filtration. The solid was reconstituted in DCM and washed with brine (1×). The organic layer was dried with sodium sulfate and concentrated via rotary evaporation. The crystalline white solid was used without further purification (40.2 g, 251 mmol, 77%). ¹H NMR (500 MHz, CDCl₃) δ 9.91 (s, 1H), 7.86 (d, J=8.7 Hz, 2H), 7.10 (d, J=8.8 Hz, 2H), 4.78 (d, J=2.4 Hz, 2H), 2.57 (t, J=2.4 Hz, 1H). ¹³C NMR (125 MHz, CDCl₃) δ 191.0, 162.6, 132.1, 130.8, 115.4, 77.8, 76.6, 56.2. ESI HR-MS: Calculated for C₁₀H₉O₂ [M-H]⁺ m/z 161.0603, found 161.0603.

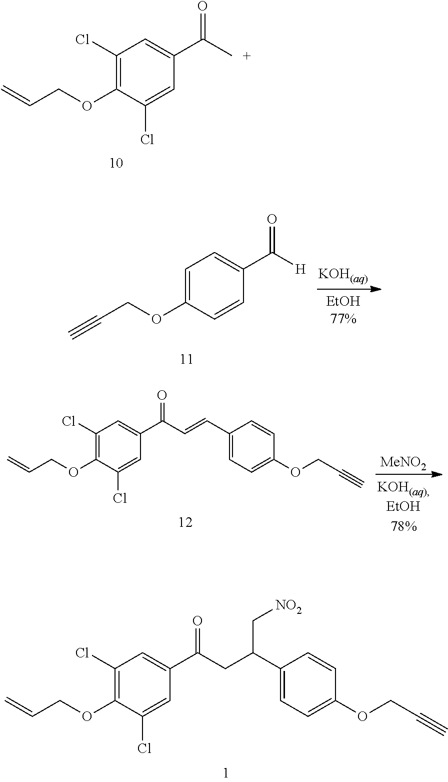

(E)-1-(4-(Allyloxy)-3,5-dichlorophenyl)-3-(4-(prop-2-yn-1-yloxy)phenyl) prop-2-en-1-one (12). To a round-bottom flask, 10 (5.0 g, 20.4 mmol, 1 equiv.) and 11 (3.3 g, 20.4 mmol, 1 equiv.) were dissolved in 200 proof ethanol (50 mL). The reaction mixture was stirred at high-speed. Then, aqueous 10 M KOH (6.1 mL, 61.2 mmol, 3 equiv.) was added dropwise. The product began to crash out of solution within 5 minutes. The reaction was stirred for 3 hours at room temperature. The resulting solid was isolated via vacuum filtration and washed with cold 200 proof ethanol (1×). The yellow-white solid was used without further purification (6.0 g, 15.6 mmol, 77%). ¹H NMR (500 MHz, CDCl₃) δ 7.95 (s, 2H), 7.81 (d, J=15.5 Hz, 1H), 7.63 (d, J=8.7 Hz, 2H), 7.31 (d, J=15.6 Hz, 1H), 7.03 (d, J=8.8 Hz, 1H), 6.21-6.10 (m, 1H), 5.45 (dd, J=17.1, 1.5 Hz, 1H), 5.31 (dt, J=10.2, 1.2 Hz, 1H), 4.76 (d, J=2.4 Hz, 2H), 4.66 (dt, J=6.1, 1.3 Hz, 2H), 2.58-2.54 (m, 1H). ¹³C NMR (125 MHz, CDCl₃) δ 186.9, 159.9, 154.7, 145.7, 135.3, 132.5, 130.4, 130.1, 129.0, 128.0, 119.4, 118.7, 115.4, 77.9, 76.1, 74.6, 55.8.

1-(4-(Allyloxy)-3,5-dichlorophenyl)-4-nitro-3-(4-(prop-2-yn-1-yloxy)phenyl) butan-1-one (1). To a round-bottom flask, 12 (5.3 g, 13.7 mmol, 1 equiv.) and nitromethane (11.1 mL, 206.2 mmol, 15 equiv.) were suspended in 200 proof ethanol (28 mL). The mixture was treated dropwise with 10 M KOH (0.27 μL, 2.7 mmol, 0.2 equiv.) and was allowed to stir at room temperature overnight. The reaction was quenched with water and extracted with EtOAc (3×). The combined organic layers were dried with sodium sulfate and concentrated via rotary evaporation. The crude oil was purified via column chromatography (SiO₂, 1:3 v/v EtOAc:Hexanes->1:1 v/v EtOAc:Hexanes) to afford the product as an off-white solid (4.8 g, 11 mmol, 78%). ¹H NMR (500 MHz, CDCl₃) § 7.83 (s, 2H), 7.20 (d, J=8.8 Hz, 2H), 7.00-6.91 (m, 2H), 6.12 (ddt, J=16.5, 10.4, 6.1 Hz, 1H), 5.40 (d, J=1.4 Hz, 1H), 5.30 (dt, J=10.4, 1.3 Hz, 1H), 4.81-4.72 (m, 1H), 4.69-4.51 (m, 4H), 4.14 (dt, J=11.9, 7.1 Hz, 1H), 3.42-3.29 (m, 2H), 2.52 (t, 1H). ¹³C NMR (125 MHz, CDCl₃) δ 193.8, 157.2, 155.4, 133.1, 132.3, 131.5, 130.3, 128.7, 128.5, 119.5, 115.4, 115.1, 79.5, 78.3, 75.7, 74.7, 60.4, 55.8, 41.5, 38.4. ESI HR-MS: Calculated for C₂₂H₂₀O₅Cl₂ [M-H]⁺ m/z 448.0719, found 448.0698.

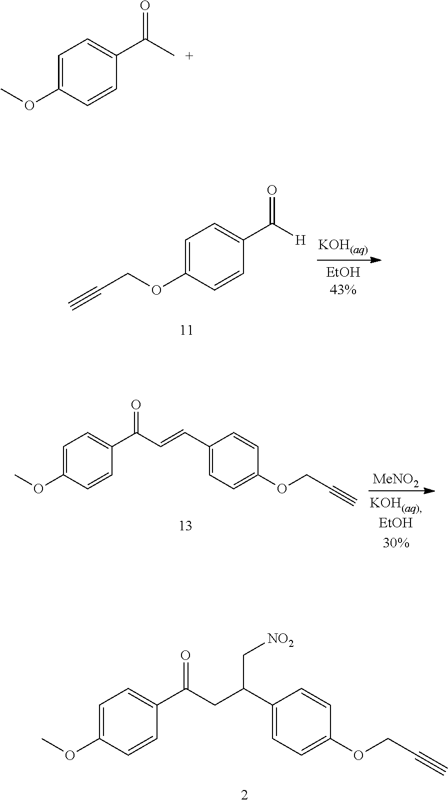

(E)-1-(4-Methoxyphenyl)-3-(4-(prop-2-yn-1-yloxy)phenyl) prop-2-en-1-one (13). To a round-bottom flask, 4-methoxyacetophenone (9.6 g, 64.2 mmol, 1 equiv.) and 11 (12.3 g, 77.0 mmol, 1.2 equiv.) were dissolved in 200 proof ethanol (155 mL). The solution was rapidly stirred, and then 10 M KOH (19.2 mL, 192.6 mmol, 3 equiv.) was added dropwise. Solid began to precipitate from the reaction, and it was allowed to stir at room temperature overnight. The solid was isolated via vacuum filtration and washed with cold ethanol (1×). The resulting off-white solid was used without further purification (8.1 g, 28 mmol, 43%). ¹H NMR (500 MHz, CDCl₃) δ 8.03 (d, J=8.8 Hz, 2H), 7.78 (d, J=15.6 Hz, 1H), 7.62 (d, J=8.8 Hz, 2H), 7.44 (d, J=15.6 Hz, 1H), 7.00 (dd, J=17.6, 8.8 Hz, 5H), 4.75 (d, J=2.4 Hz, 3H), 3.89 (s, 4H), 2.58-2.53 (m, 2H). ¹³C NMR (125 MHz, CDCl₃) δ 188.7, 163.3, 159.3, 143.5, 131.3, 130.7, 130.6, 130.0, 128.6, 123.9, 120.0, 115.3, 115.30, 113.8, 113.6, 78.0, 75.9, 55.8, 55.5, 26.3. ESI HR-MS: Calculated for C₁₉H₁₇O₃ [M-H]⁺ m/z 293.1178, found 293.1177.

1-(4-Methoxyphenyl)-4-nitro-3-(4-(prop-2-yn-1-yloxy)phenyl) butan-1-one (2). To a round-bottom flask, 13 (11.8 g, 40.5 mmol, 1 equiv.) and nitromethane (32.8 mL, 607.4 mmol, 15 equiv.) were suspended in ethanol (80 mL). The mixture was treated dropwise with 10 M KOH (0.81 μL, 8.1 mmol, 0.2 equiv.) and was allowed to stir at room temperature overnight. The reaction was quenched with water and extracted with EtOAc (3×). The combined organic layers were dried with sodium sulfate and concentrated via rotary evaporation. The crude oil was purified via column chromatography (SiO₂, 1:3 v/v EtOAc:Hexanes) to afford the product as an off-white solid or yellow oil (4.3 g, 12.1 mmol, 30%). ¹H NMR (500 MHz, CDCl₃) δ 7.93-7.86 (d, 2H), 7.25-7.18 (d, 2H), 6.97-6.89 (m, 4H), 4.81 (dd, J=12.4, 6.5 Hz, 1H), 4.66 (d, J=2.5 Hz, 2H), 4.65-4.61 (m, 1H), 4.22-4.12 (m, 1H), 3.87 (s, 3H), 3.43-3.29 (m, 2H), 2.51 (t, J=2.4 Hz, 1H). ¹³C NMR (125 MHz, CDCl₃) δ 195.5, 164.0, 157.2, 132.3, 130.5, 129.7, 128.7, 115.5, 114.1, 80.0, 78.6, 75.8, 56.0, 55.7, 41.4, 38.9. ESI HR-MS: Calculated for C₂₀H₂₀NO₅ [M-H]⁺ m/z 354.1341, found 354.1339

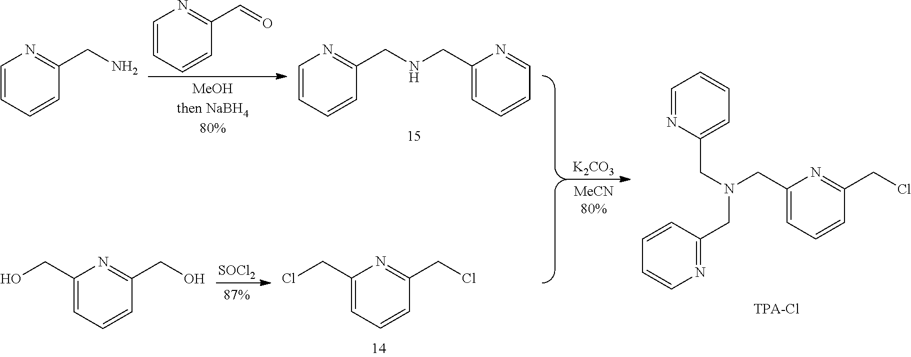

2,6-Bis(chloromethyl)pyridine (14). To a round-bottom flask, 2,6-pyridinemethanol (5.2 g, 37 mmol, 1 equiv.) was cooled to 0° C. and treated dropwise with thionyl chloride (52.8 mL, 723.4 mmol, 19.5 equiv.). The reaction was allowed to warm to room temperature and stirred for 6 hours. The reaction was cooled to 0° C. and quenched via dropwise addition of aqueous ammonium hydroxide. The solid precipitate was isolated via vacuum filtration and allowed to dry for 3 hours. The white solid was used without further purification (5.7 g, 32.3 mmol, 87%). ¹H NMR (500 MHz, CDCl₃) δ 7.69 (t, J=7.8 Hz, 1H), 7.37 (d, J=7.7 Hz, 2H), 4.59 (s, 4H). ¹³C NMR (125 MHz, CDCl₃) δ 156.8, 138.6, 122.5, 46.9. ESI HR-MS: Calculated for C₇H₈NCl₂ [M-H]⁺ m/z 176.0034, found 176.0036.

Bis(pyridin-2-ylmethyl)amine (15). To a round-bottom flask, 2-aminomethylpyridine (7.6 g, 70.0 mmol, 1 equiv.) was dissolved in MeOH (140 mL). Then, 2-pyridinecarboxaldehyde (7.5 g, 70.0 mmol, 1 equiv.) was added. The reaction mixture became a dark brown color immediately upon addition of the aldehyde, and the reaction was allowed to stir at room temperature overnight to enable imine formation. Then, sodium borohydride (5.3 g, 140.0 mmol, 2 equiv.) was added portion-wise to the reaction. The mixture turned yellow, and the reaction was allowed to stir for another 2 hours at room temperature. The reaction was concentrated via rotary evaporation and then quenched via addition of water and acidified with 1 M HCl. The aqueous solution was extracted with DCM (3×). The combined organic layers were dried with sodium sulfate and concentrated via rotary evaporation. The yellow residue was purified through a silica plug (SiO₂, 1:19 v/v MeOH:DCM) to afford the product as a light yellow liquid (11.1 g, 55.8 mmol, 80%). ¹H NMR (500 MHz, CDCl₃) δ 8.56 (d, J=5.0 Hz, 2H), 7.70 (t, J=8.0 Hz, 2H), 7.45 (d, J=7.8 Hz, 2H), 7.27-7.16 (m, 2H), 5.55 (s, 1H), 4.33 (s, 4H). ¹³C NMR (125 MHz, CDCl₃) δ 152.8, 149.4, 137.2, 123.4, 123.1, 51.6. ESI HR-MS: Calculated for C₁₂H₁₄N₃ [M-H]⁺ m/z 200.1188, found 200.1189.

TPA-Cl. To a round-bottom flask, 15 (1.0 g, 5.1 mmol, 1 equiv.) was dissolved in acetonitrile (100 mL). Then, 14 (3.1 g, 17.8 mmol, 3.5 equiv.) and potassium carbonate (771.2 mg, 5.6 mmol, 1.1 equiv.) were added sequentially. The reaction was heated to 60° C. and allowed to stir overnight. The reaction was cooled to room temperature, and the solids were removed via vacuum filtration. The filtrate were concentrated via rotary evaporation, and the crude residue was purified via column chromatography (SiO₂, 1:19 v/v MeOH:DCM) to afford the product as an off-white solid (1.4 g, 4.1 mmol, 80%). ¹H NMR (500 MHz, CDCl₃) δ 8.54 (dt, J=4.8, 1.3 Hz, 2H), 7.72-7.63 (m, 4H), 7.59 (d, J=7.8 Hz, 2H), 7.54 (d, J=7.7 Hz, 1H), 7.33 (d, J=7.6 Hz, 1H), 7.15 (ddd, J=7.6, 4.9, 1.3 Hz, 2H), 4.64 (s, 2H), 3.95 (s, 4H). ¹³C NMR (125 MHz, CDCl₃) δ 158.8, 155.9, 149.0, 137.49, 136.6, 123.1, 122.2, 122.1, 121.1, 60.0, 59.8, 46.8. ESI HR-MS: Calculated for C₁₉H₂₀N₄Cl [M-H]⁺ m/z 339.1376, found 339.1372.

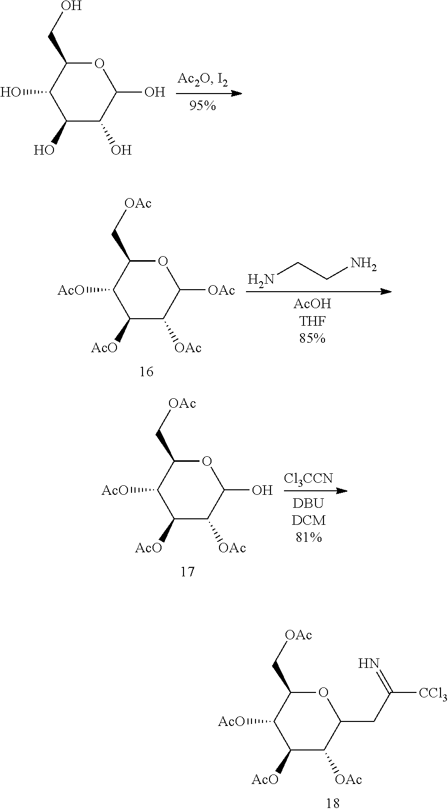

1,2,3,4,6-penta-O-acetyl-n-glucopyranose (16). Iodine (50 mg, 0.4 mmol, 0.035 equiv.) was dissolved in acetic anhydride (18.7 mL). d-Glucose (2 g, 11 mmol, 1 equiv.) was slowly added. The reaction was allowed to stir at room temperature overnight. The reaction was quenched with aqueous potassium thioacetate. After the reaction turned a milky, light yellow color, the reaction was concentrated by rotary evaporation. After the majority of excess acetic anhydride was removed, the reaction was diluted with aqeuous sodium bicarbonate until a pH of 8 was reached. Then the aqeous layer was extracted with DCM (4×). The combined organic layers were dried with sodium sulfate and concentrated via rotary evaporation. The resulting solid was used without further purification (95%, 4.1 g, 10.5 mmol). ¹H NMR (500 MHz, CDCl₃) δ 6.33 (d, J=3.7 Hz, 1H), 5.47 (t, J=9.9 Hz, 1H), 5.18-5.07 (m, 2H), 4.27 (dd, J=12.4, 4.1 Hz, 1H), 4.16-4.06 (m, 2H), 2.18 (s, 3H), 2.10 (s, 4H), 2.06-2.00 (m, 10H). ¹³C NMR (125 MHz, CDCl₃) δ 170.6, 170.2, 169.6, 169.3, 168.7, 89.0, 69.8, 69.1, 67.8, 61.4, 20.8, 20.7, 20.6, 20.5, 20.4. ESI HR-MS: Calculated for NaC₁₆H₂₂O₁₁ [M]⁺ m/z 413.1060, found 413.1051.

2,3,4,6-tetra-O-acetyl-n-glucopyranose (17). Ethylenediamine (0.1 mL, 1.5 mmol, 1.2 equiv.) was added to THF (8 mL). Acetic acid (0.1 mL, 1.8 mmol, 1.4 equiv.) was added dropwise over the period of 10 minutes, and the suspension was allowed to stir at room temperature for 1 hour. Then, 16 (500 mg, 1.3. 1 equiv.) dissolved in THF (2 mL) was added to the reaction and allowed to stir at room temperature overnight. The reaction was quenched with addition of 1 M HCl and extracted with ethyl acetate (4×). The combined organic layers were washed with aqeous NaHCO₃, dried with sodium sulfate, and concentrated via rotary evaporation. The resulting oil was purified via flash column chromatography (SiO₂, 1:1 v/v EtOAc:Hexanes) to give the desired product as an off-white solid (90%, 1.3 g, 3.8 mmol). ¹H NMR (500 MHz, CDCl₃) δ 5.47 (t, J=9.8 Hz, 1H), 5.40 (d, J=3.4 Hz, 1H), 5.02 (s, 1H), 4.87-4.78 (m, 1H), 4.24-4.14 (m, 2H), 4.12-4.03 (m, 1H), 3.24 (s, 1H), 2.03 (s, 3H), 2.02-1.94 (m, 9H). ¹³C NMR (125 MHz, CDCl₃) δ 170.8, 170.2, 170.1, 169.6, 169.6, 90.1, 71.0, 71.0, 69.8, 68.4, 68.4, 67.2, 61.9, 20.7, 20.7, 20.7, 20.6.

2,3,4,6-tetra-O-acetyl-n-glucopyranosyl trichloroacetimidate (18). 17 (380 mg, 1.1 mmol, 1 equiv.) was dissolved in DCM (2.3 mL) and cooled to 0° C. Then, trichloroacetonitrile (1.1 mL, 11 mmol, 10 equiv.) and DBU (41 μL, 0.3 mmol, 0.25 equiv.) were added sequentially to the reaction. The reaction was warmed to room temperature and was stirred overnight. The reaction was concentrated via rotary evaporation and the resulting oil was purified via flash column chromatography (SiO₂, 1:1 v/v EtOAc:Hexanes) to give the desired product as a pale yellow oil (81%, 435 mg, 0.9 mmol). ¹H NMR (500 MHz, CDCl₃) δ 8.69 (s, 1H), 6.56 (d, J=3.7 Hz, 1H), 5.57 (t, J=9.8 Hz, 1H), 5.22-5.10 (m, 2H), 4.28 (dd, J=12.4, 4.2 Hz, 1H), 4.22 (dt, J=10.7, 3.0 Hz, 1H), 4.13 (dd, J=12.5, 2.2 Hz, 1H), 2.12-1.95 (m, 12H). ¹³C NMR (125 MHz, CDCl₃) δ 171.1, 170.5, 170.0, 169.8, 169.5, 160.8, 92.9, 90.6, 70.0, 69.8, 69.7, 67.7, 61.3, 60.4, 21.0, 20.6, 20.6, 20.4, 14.2. ESI HR-MS: Calculated for NaC₁₆H₂₀NO₁₀Cl₃ [M−Na]⁺ m/z 514.0050, found 514.0041.

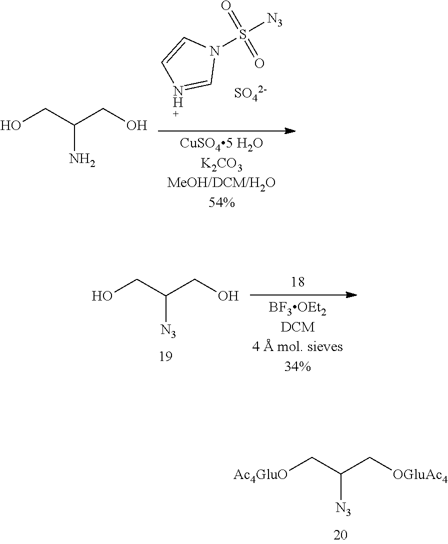

1-(Azidosulfonyl)-1H-imidazol-3-ium hydrogen sulfate. CAUTION: Intermediates in the synthesis of the azide transfer salt are explosive under reduced pressure and solutions must not be rotovaped. A flame-dried, two-neck flask was evacuated and charged with N₂. Sodium azide (10.0 g, 154 mmol, 1 equiv.) and ethyl acetate dried over 4 Å molecular sieves (154 mL, 1.0 M) were added and cooled to 0° C. Sulfuryl chloride (12.51 mL, 154 mmol, 1 equiv.) was added dropwise over a period of 10 minutes. The reaction was warmed slowly to room temperature and was allowed to stir for 17 hours. The reaction was cooled to 0° C. and imidazole (19.9 g, 292 mmol, 1.9 equiv.) was added portionwise and was allowed to stir at 0° C. for 3 hours. The reaction was neutralized via the addition of aqueous sodium bicarbonate (pH=8). The EtOAc was separated from the aqueous layer. The aqueous layer was extracted with EtOAc (150 mL), and both organic layers were combined, dried with sodium sulfate, and transferred into a 500 mL round-bottom flask. Due to the HCl salt being explosive, the organic layer cannot be rotovaped. The organic solution could be stored under N₂ at −20° C. overnight if needed. N₂ was blown over the dried organic layer until ˜150 mL of EtOAc was remaining. The solution was cooled to 0° C., and a stirbar was added and set to vigorous stirring. Sulfuric acid (8.28 mL) was then added dropwise over a period of 5 minutes. The solution was warmed to room temperature and was allowed to stir for 1 hour before being stored at −20° C. overnight. The white precipitate was filtered and washed with minimal cold EtOAc. The white solid (36 g) was stored under N₂ at −20° C. and used without further purification.

2-Azidopropane-1,3-diol (19). 2-Aminopropane-1,3-diol (5.8 g, 63.7 mmol, 1 equiv.) was dissolved in 2:1:1 v/v MeOH:DCM:Water (320:160:160 mL). Potassium carbonate (17.6 g, 127.4 mmol, 2 equiv.) and CuSO₄·5H₂O (159.1 mg, 0.64 mmol, 0.01 equiv.) were added, and the reaction mixture was allowed to stir at room temperature for 5 minutes. 1-(Azidosulfonyl)-1H-imidazol-3-ium hydrogen sulfate (18.9 g, 70.1 mmol, 1.1 equiv.) was then added portionwise, and the reaction was kept under N₂ and was allowed to stir at room temperature overnight. The reaction was acidified via the addition of 1 M HCl until pH=2. The aqeous layer was extracted with EtOAc (3×), dried with sodium sulfate, and concentrated via rotary evaporation. The product (4.0 g, 34.4 mmol, 54%) was used without further purification. ¹H NMR (500 MHz, CDCl₃) δ 3.86 (dd, J=11.5, 4.5 Hz, 2H), 3.80 (dd, J=11.4, 5.9 Hz, 2H), 3.69 (p, J=5.3 Hz, 1H), 1.93 (s, 3H). ¹³C NMR (125 MHz, MeOD) δ 66.6, 62.7.

Compound 20. A round-bottom flask containing activated 4 Å molecular sieves was charged with 19 (1.4 g, 12.0 mmol, 1 equiv.), 18 (14.7 g, 29.9 mmol, 2.5 equiv.), and DCM (115 mL). The reaction was cooled to 0° C., and BF₃·OEt₂ (0.151 mL, 1.2 mmol, 0.1 equiv.) diluted with DCM (5 mL) was added dropwise to the reaction. The reaction was slowly warmed to room temperature and stirred overnight. The reaction was filtered over a pad of celite, concentrated via rotory evaporation, and purified via column chromatography (SiO₂, 1:1 v/v EtOAc:Hexanes) to afford the desired product as a fluffy white solid (3.2 g, 4.1 mmol, 34%). ¹H NMR (500 MHz, CDCl₃) δ 5.24-5.17 (m, 2H), 5.14-4.95 (m, 4H), 4.56 (d, J=7.9 Hz, 1H), 4.52 (d, J=8.0 Hz, 1H), 4.26 (dt, J=11.5, 5.7 Hz, 2H), 4.15 (dd, J=13.5, 11.2 Hz, 3H), 4.03 (dd, J=10.1, 3.3 Hz, 1H), 3.88 (dd, J=10.6, 4.7 Hz, 1H), 3.71 (s, 3H), 3.66-3.57 (m, 2H), 2.12-1.99 (m, 30H). ¹³C NMR (125 MHz, CDCl₃) δ 170.7, 170.6, 170.1, 170.0, 169.6, 169.4, 95.6, 91.3, 90.2, 73.2, 72.1, 72.0, 70.9, 69.9, 69.7, 69.6, 68.4, 67.3, 67.3, 61.9, 61.5, 20.9, 20.8, 20.7, 20.7, 20.7, 20.6, 20.6. ESI HR-MS: Calculated for NaC₃₁H₄₃N₃O₂₀ [M−Na]⁺ m/z 800.2338, found 800.2338.

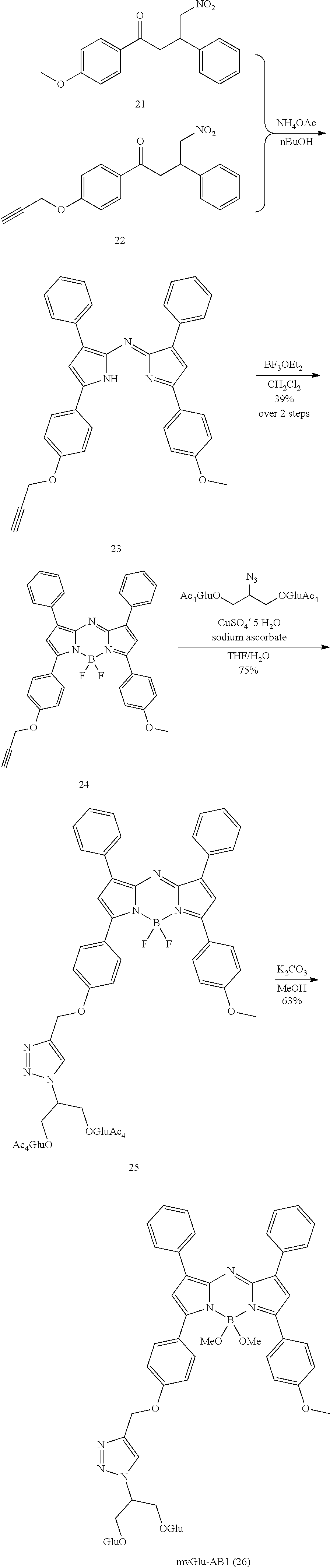

Compound 24. To a round-bottom flask, 21 (1.0 g, 3.3 mmol, 1 equiv.) and 22 (2.2 g, 6.7 mmol, 2 equiv.) were suspended in n-butanol (20 mL). The solution was heated to 130° C. for 45 minutes in an oil bath containing rice bran oil. Then, ammonium acetate (3.9 g, 50 mmol, 15 equiv.) was added, and the reaction was allowed to heat at 130° C. for 5 hours. The reaction was cooled to room temperature, diluted with water, and extracted with DCM (3×). The organic layers were combined, dried with sodium sulfate, and concentrated via rotary evaporation. The excess n-butanol was removed azeotropically with toluene. The resulting shiny blue solid was used without further purification. The solid was dissolved in DCM (330 mL) and cooled to 0° C. DIPEA (5.8 mL, 33 mmol, 10 equiv.) and BF₃·OEt₂ (5.9 mL, 47 mmol, 14 equiv.) were added sequentially. The reaction was warmed to room temperature and was allowed to stir overnight. The reaction was quenched with aqueous sodium bicarbonate and extracted with DCM (3×). The combined organic layers were dried with sodium sulfate and concentrated via rotary evaporation. The resulting solid was purified via column chromatography (SiO₂, toluene) to afford the desired compound as a green solid (751 mg, 1.3 mmol, 39%). ¹H NMR (500 MHz, CDCl₃) δ 8.07-7.93 (m, 8H), 7.52-7.30 (m, 6H), 7.02 (d, J=9.0 Hz, 2H), 6.99 (d, J=1.2 Hz, 1H), 6.95 (d, J=9.0 Hz, 3H), 4.70 (d, J=2.4 Hz, 2H), 3.82 (s, 3H), 2.50 (t, J=2.4 Hz, 1H). ¹³C NMR (125 MHz, CDCl₃) δ 162.0, 159.7, 132.5, 132.4, 131.7, 131.5, 129.3, 129.2, 129.2, 128.5, 128.4, 125.0, 124.0, 118.8, 118.5, 115.0, 114.3, 78.0, 76.0, 55.8, 55.4. ESI HR-MS: Calculated for C₃₆H₂₆N₃BO₂F₂ [M]⁺ m/z 581.2086, found 581.2086.

Compound 25. To a round-bottom flask, 24 (50.0 mg, 0.086 mmol, 1 equiv.), 20 (167.2 mg, 0.22 mmol, 2.5 equiv.), and CuSO₄·H₂O (215 mg, 0.86 mmol, 10 equiv.) were dissolved in THF (4.3 mL). Sodium ascorbate (86.9 mg, 0.44 mmol, 5.1 equiv.) was dissolved in water (0.87 mL) and was added to the reaction suspension dropwise. The reaction was allowed to stir at room temperature overnight. The reaction was diluted with water and extracted with EtOAc (3×). The combined organic layers were dried with sodium sulfate and concentrated via rotary evaporation. The resulting blue oil was purified via column chromatography (SiO₂, 4:1 v/v EtOAc:Hexane then 1:19 v/v MeOH:DCM) to afford the desired compound as a green solid (87.2 mg, 0.064 mmol, 75%). ¹H NMR (500 MHz, Chloroform-d) δ 8.08 (dd, J=19.3, 7.8 Hz, 4H), 7.78 (q, J=9.0, 7.4 Hz, 1H), 7.44 (dt, J=15.4, 6.9 Hz, 4H), 7.17-6.93 (m, 4H), 5.18 (q, J=9.2 Hz, 3H), 5.06 (qd, J=9.7, 8.3, 4.8 Hz, 2H), 5.01-4.88 (m, 3H), 4.48 (ddd, J=24.4, 11.9, 5.5 Hz, 3H), 4.25 (tt, J=19.1, 14.9, 5.9 Hz, 5H), 4.18-4.09 (m, 3H), 4.06-3.95 (m, 1H), 3.89 (d, J=6.1 Hz, 2H), 3.74-3.65 (m, 4H), 2.00 (td, J=9.6, 8.8, 4.2 Hz, 24H). ¹³C NMR (126 MHz, CDCl₃) δ 170.6, 170.1, 169.4, 169.4, 169.4, 143.2, 137.2, 131.7, 129.3, 128.5, 124.1, 115.0, 114.3, 100.9, 97.5, 94.9, 90.4, 72.4, 72.0, 71.9, 70.9, 68.3, 68.2, 68.1, 61.7, 61.6, 55.4, 29.7, 20.7, 20.6, 20.6, 20.5, 20.5. ESI HR-MS: Calculated for C₆₇H₆₉N₆BO₂₂F₂ [M]⁻ m/z 1358.4526, found 1358.4556.

mvGlu-AB1 (26). To a round-bottom flask, 25 (24.5 mg, 0.018 mmol, 1 equiv.) and potassium carbonate (46.8 mg, 0.34 mmol, 18.8 equiv.) were suspended in methanol (1.8 mL). The reaction was stirred at room temperature for 3 hours. Then, the solvent was removed via rotary evaporation and purified via reverse-phase chromatography (C18, 100% H₂O→50% Acetonitrile/H₂O) to afford the product as a green solid (11.9 mg, 0.011 mmol, 63%). ¹H NMR (500 MHz, Acetonitrile-d₃) δ 8.15 (s, 4H), 8.05-8.00 (m, 5H), 7.40 (d, J=22.8 Hz, 8H), 7.19 (s, 2H), 7.08 (s, 2H), 6.97 (d, J=7.8 Hz, 3H), 5.20 (s, 2H), 5.10 (s, 2H), 4.31 (dd, J=10.8, 7.9 Hz, 2H), 4.15-4.01 (m, 3H), 3.81 (s, 4H), 3.75 (dd, J=12.4, 7.0 Hz, 3H), 3.55 (dd, J=12.3, 6.0 Hz, 3H), 3.37-3.24 (m, 6H), 3.21 (q, J=9.4, 7.3 Hz, 3H), 3.11 (t, J=8.6 Hz, 3H), 2.74 (s, 7H). ¹³C NMR (126 MHz, CD₃CN) δ 163.9, 158.9, 144.4, 133.2, 133.2, 129.9, 129.6, 119.4, 115.4, 114.8, 103.8, 103.4, 88.6, 76.9, 76.6, 73.9, 70.6, 70.6, 61.9, 61.8, 56.3, 49.0. ESI HR-MS: Calculated for C53H₅₈BN₆O₁₆ [M-H]⁻ m/z 1045.4002, found 1045.4014.

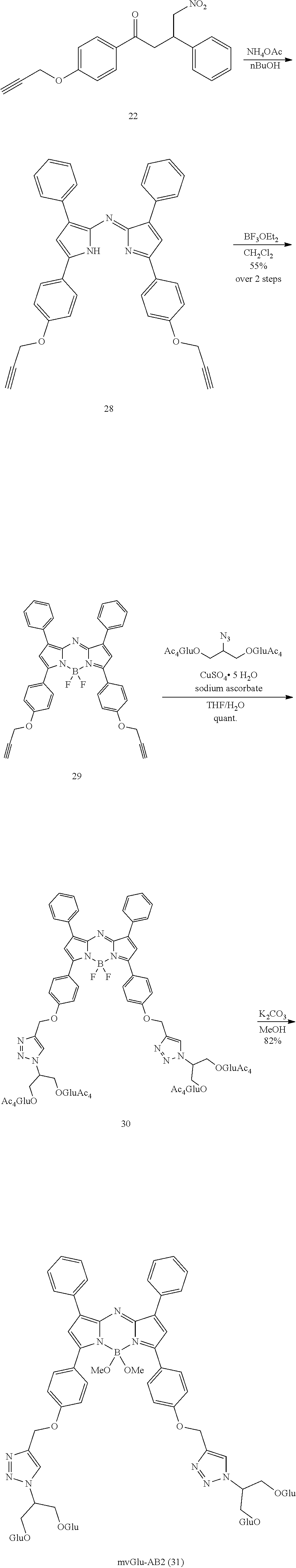

Compound 29. To a round-bottom flask, 22 (7.0 g, 21.6 mmol, 1 equiv.) and ammonium acetate (25.0 g, 324 mmol, 15 equiv.) were suspended in n-butanol (455 mL). The solution was heated to 130° for 5 hours. The reaction was cooled to room temperature, diluted with water, and extracted with DCM (3×). The organic layers were combined, dried with sodium sulfate, and concentrated via rotary evaporation. The excess n-butanol was removed azeotropically with toluene. The resulting shiny blue solid was used without further purification. The solid was dissolved in DCM (200 mL) and cooled to 0° C. DIPEA (31 mL, 180 mmol, 15 equiv.) and BF₃·OEt₂ (67 mL, 534 mmol, 45 equiv.) were added sequentially. The reaction was warmed to room temperature and was allowed to stir overnight. The reaction was quenched with aqueous sodium bicarbonate and extracted with DCM (3×). The combined organic layers were dried with sodium sulfate and concentrated via rotary evaporation. The resulting solid was purified via column chromatography (SiO₂, toluene) to afford the desired compound as a shiny green solid (3.6 g, 5.9 mmol, 55%). ¹H NMR (500 MHz, Chloroform-d) 8 8.17-8.03 (m, 8H), 7.57-7.40 (m, 6H), 7.13-7.06 (m, 4H), 7.04 (d, J=1.3 Hz, 2H), 4.78 (d, J=2.4 Hz, 3H), 2.57 (t, J=2.4 Hz, 2H). ¹³C NMR (125 MHz, CDCl₃) δ 159.8, 132.4, 131.5, 129.3, 128.6, 124.9, 118.7, 115.0, 107.7, 78.0, 76.0, 55.8. ESI HR-MS: Calculated for C₃₈H₂₆BN₃O₂F₂ [M]⁻ m/z 605.2086, found 605.2094.

Compound 30. In a round bottom flask, 29 (75 mg, 124 μmol, 1 equiv.), 20 (385 mg, 495 μmol, 4 equiv.), and CuSO₄·5H₂O (309 mg, 1240 μmol, 10 equiv.) were suspended in THF (6.2 mL). Sodium ascorbate (125 mg, 632 μmol, 5.1 equiv.) was dissolved in DI water (1.25 mL) and added dropwise to the reaction mixture. The reaction was allowed to stir under N₂ for 3 hours at room temperature. The reaction mixture was concentrated via rotary evaporation and purified via column chromatography (SiO₂, 1:19 v/v MeOH:DCM) to afford a green solid (267 mg, 124 μmol, 100%). ¹H NMR (500 MHz, CDCl₃) δ 8.12 (d, J=8.5 Hz, 4H), 8.07 (d, J=7.5 Hz, 4H), 7.79 (s, 2H), 7.45 (dt, J=14.8, 7.1 Hz, 6H), 7.14 (d, J=8.5 Hz, 4H), 7.07 (s, 2H), 5.53 (d, J=9.8 Hz, 1H), 5.28 (s, 4H), 5.18 (td, J=9.5, 6.0 Hz, 4H), 5.13-4.91 (m, 11H), 4.49 (dd, J=17.1, 7.9 Hz, 4H), 4.28-4.17 (m, 10H), 4.10-3.96 (m, 4H), 3.70 (s, 5H), 2.06-1.96 (m, 41H).

mvGlu-AB2 (31). To a round bottom flask, 30 (180 mg, 83 μmol, 1 equiv.) and potassium carbonate (216 mg, 1.56 mmol, 18.8 equiv.) were suspended in methanol (8.33 mL). The reaction was stirred at room temperature for 3 hours. Then, the solvent was removed via rotary evaporation and purified via reverse-phase chromatography (C18, 100% H₂O→50% Acetonitrile/H₂O) to afford the product as a green solid (101 mg, 68 μmol, 82%). ¹H NMR (500 MHz, 1:1 CD₃CN:D₂O) δ 8.20 (dd, J=15.8, 7.7 Hz, 4H), 8.13-7.97 (m, 5H), 7.62 (s, 2H), 7.51-7.37 (m, 5H), 7.24 (d, J=7.6 Hz, 2H), 7.12 (dd, J=17.6, 8.6 Hz, 4H), 5.22 (d, J=6.0 Hz, 4H), 5.10 (s, 3H), 4.42-4.22 (m, 7H), 4.05 (d, J=8.8 Hz, 1H), 3.84-3.73 (m, 5H), 3.55 (dd, J=12.2, 6.1 Hz, 5H), 3.31 (dt, J=21.6, 11.3 Hz, 11H), 3.24-3.18 (m, 5H), 3.11 (t, J=8.5 Hz, 5H), 2.89 (s, 1H), 2.79 (s, 3H), 2.75 (s, 1H), 2.10 (s, 2H). ¹³C NMR (125 MHz, 1:1 CD₃CN:D₂O) δ 165.2, 147.39, 133.1, 130.5, 129.1, 115.7, 115.4, 103.8, 103.4, 76.9, 76.6, 73.9, 70.6, 69.3, 68.9, 61.9, 50.7, 49.3, 37.5, 32.0, 29.3. ESI HR-MS: Calculated for C₇₀H₈₅BN₉O₂₈ [M−2H]⁻ m/z 1510.5597, found 1510.5587.

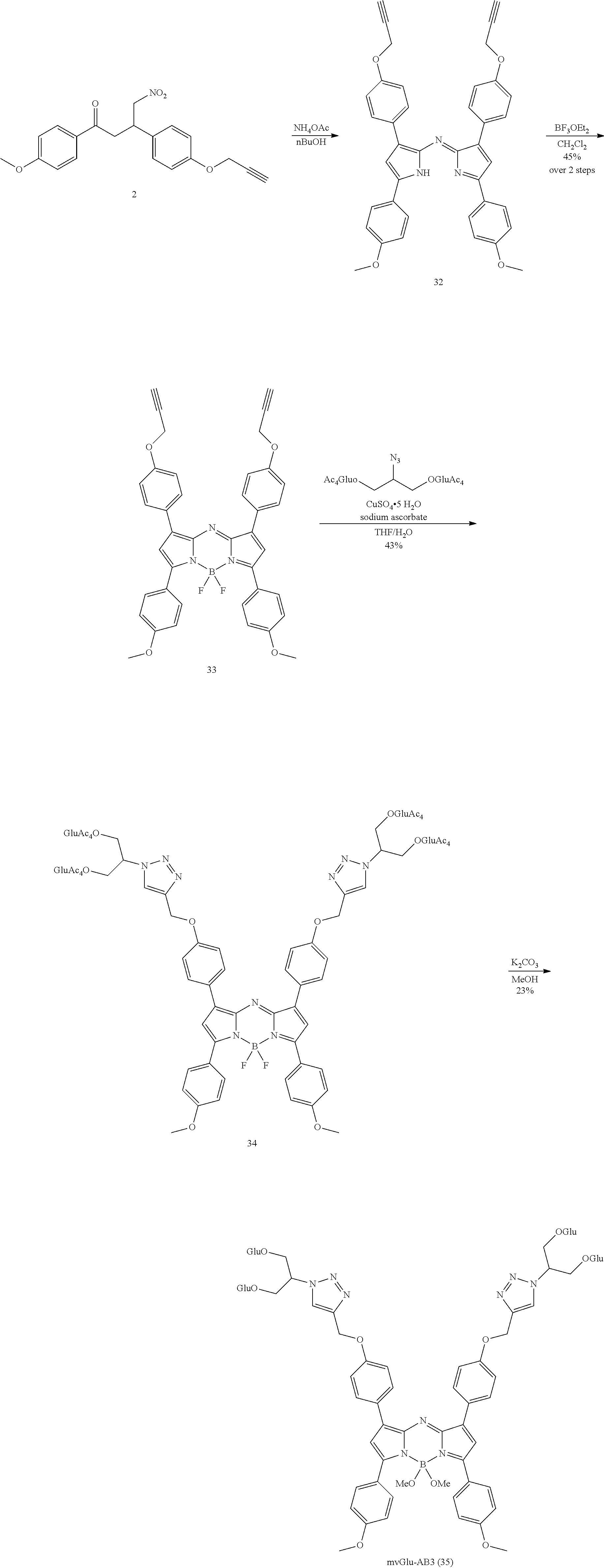

Compound 33. To a round-bottom flask, 2 (300 mg, 0.849 mmol, 1 equiv.) and ammonium acetate (490 mg, 6.37 mmol, 15 equiv.) were suspended in n-butanol (3 mL). The solution was heated to 130° for 5 hours. The reaction was cooled to room temperature, diluted with water, and extracted with DCM (3×). The organic layers were combined, dried with sodium sulfate, and concentrated via rotary evaporation. The excess n-butanol was removed azeotropically with toluene. The resulting shiny blue solid was used without further purification. The solid was dissolved in DCM (42 mL) and cooled to 0° C. DIPEA (0.739 mL, 4.2 mmol, 10 equiv.) and BF₃·OEt₂ (0.734 mL, 5.9 mmol, 14 equiv.) were added sequentially. The reaction was warmed to room temperature and was allowed to stir overnight. The reaction was quenched with aqueous sodium bicarbonate and extracted with DCM (3×). The combined organic layers were dried with sodium sulfate and concentrated via rotary evaporation. The resulting solid was purified via column chromatography (SiO₂, toluene) to afford the desired compound as a shiny blue solid (127 mg, 0.191 mmol, 45%). ¹H NMR (400 MHz, CHCl₃) δ 8.16 (dd, J=8.8, 4.2 Hz, 4H), 7.79-7.68 (m, 3H), 7.14-7.03 (m, 4H), 7.02 (s, 1H), 4.77 (d, J=2.4 Hz, 5H), 3.88 (s, 3H), 2.57 (dt, J=5.1, 2.4 Hz, 2H). ¹³C NMR (126 MHz, CDCl₃) δ 160.6, 159.4, 157.3, 157.2, 155.8, 155.6, 148.3, 132.2, 131.3, 131.2, 127.3, 127.3, 127.2, 114.3, 114.3, 114.3, 113.9, 112.9, 77.4, 77.3, 77.2, 76.2, 74.8, 74.6, 67.4, 65.3, 54.8, 54.3. ESI HR-MS: Calculated for C40H₃₁₀+N₃BF₂ [M-H]⁺ m/z 666.2376, found 666.2374.

Compound 34. In a round bottom flask, 33 (60 mg, 0.092 mmol, 1 equiv.), 20 (154 mg, 0.198 mmol, 2.2 equiv.), and CuSO₄·5 H₂O (225 mg, 0.902 mmol, 10 equiv.) were suspended in THF (4.5 mL). Sodium ascorbate (91 mg, 0.46 mmol, 5.1 equiv.) was dissolved in DI water (0.91 mL) and added dropwise to the reaction mixture. The reaction was allowed to stir under N₂ for 3 hours at room temperature. The reaction mixture was concentrated via rotary evaporation and purified via column chromatography (SiO₂, 1:19 v/v MeOH:DCM) to afford a green solid (87.2 mg, 0.039 mmol, 43%). ¹H NMR (500 MHz, CDCl₃) ¹H NMR (500 MHz, Chloroform-d) δ 8.09 (t, J=8.6 Hz, 2H), 8.04-7.87 (m, 2H), 7.82-7.61 (m, 5H), 7.08 (dq, J=15.0, 7.5 Hz, 4H), 6.93 (ddt, J=27.0, 19.7, 9.2 Hz, 4H), 7.06-6.76 (m, 2H), 5.33-4.76 (m, 5H), 4.43 (ddd, J=16.2, 9.8, 4.3 Hz, 6H), 4.19 (dq, J=12.6, 4.4 Hz, 5H), 4.07 (d, J=12.9 Hz, 8H), 4.03-3.89 (m, 3H), 3.82 (d, J=3.1 Hz, 3H), 3.64 (s, 10H), 1.97-1.89 (m, 48H). ¹³C NMR (125 MHz, CDCl₃) δ 170.6, 170.1, 169.4, 169.4, 169.4, 169.3, 160.5, 143.2, 131.7, 129.3, 128.5, 123.4, 115.0, 100.9, 100.8, 77.2, 72.4, 72.0, 71.9, 70.9, 68.2, 68.1, 61.9, 61.7, 61.6, 60.3, 20.7, 20.6, 20.5, 20.5, 20.5.

mvGlu-AB3 (35). To a round bottom flask, 34 (50 mg, 0.023 mmol, 1 equiv.) and potassium carbonate (58 mg, 0.42 mmol, 18.8 equiv.) were suspended in methanol (2.3 mL). The reaction was stirred at room temperature for 3 hours. Then, the solvent was removed via rotary evaporation and purified via reverse-phase chromatography (C18, 100% H₂O→50% Acetonitrile/H₂O) to afford the product as a green solid (10.2 mg, 0.0066 mmol, 23%). ¹H NMR (500 MHz, DMSO-d₆+toluene-d₈) δ 7.99 (t, J=7.5 Hz, 4H), 7.94-7.85 (m, 5H), 5.97-5.62 (m, 8H), 5.28 (s, 2H), 4.99 (d, J=8.0 Hz, 1H), 4.94-4.85 (m, 4H), 4.77 (dq, J=12.8, 7.7, 5.9 Hz, 1H), 4.60 (d, J=9.8 Hz, 1H), 4.43 (dd, J=15.5, 9.1 Hz, 2H), 4.34 (d, J=6.2 Hz, 2H), 3.90 (s, 5H), 3.79 (dt, J=8.5, 4.4 Hz, 1H), 3.76-3.67 (m, 2H) ¹³C NMR (126 MHz, DMSO-d₆+toluene-d₈) δ 163.3, 159.5, 157.5, 156.6, 153.6, 153.2, 128.9, 128.2, 103.3, 103.2, 76.9, 76.5, 76.5, 73.3, 70.0, 67.9, 67.7, 67.0, 61.1, 61.0, 60.3, 55.2, 54.9. ESI HR-MS: Calculated for C₇₂H₈₉BN₉O₃₀ [M−2H]⁻ m/z 1570.5808, found 1570.5790.

L-mvGlu-AB2 (36). L-mvGlu-AB2 was synthesized in a similar manner to what is reported for mvGlu-AB2 to afford an overall yield from 29 (55 mg, 36.9 mmol, 30%). ¹H NMR (500 MHz, 1:1 v/v D₂O:CH₃CN) δ 8.25-8.16 (m, 4H), 8.10-8.05 (m, 2H), 7.89 (dd, J=16.6, 7.9 Hz, 1H), 7.49-7.37 (m, 4H), 7.32 (dd, J=11.6, 7.0 Hz, 1H), 7.17-7.07 (m, 3H), 5.21 (d, J=21.3 Hz, 3H), 5.10 (p, J=6.4 Hz, 2H), 4.37-4.20 (m, 6H), 3.75 (ddd, J=12.2, 7.2, 2.3 Hz, 4H), 3.55 (ddd, J=12.2, 6.0, 2.2 Hz, 4H), 3.37-3.17 (m, 9H), 3.15-3.07 (m, 3H), 2.78 (s, 3H). ¹³C NMR (125 MHz, 1:1 v/v D₂O:CH₃CN) δ 167.6, 161.0, 156.6, 143.0, 137.2, 133.2, 130.0, 129.6, 115.5, 103.8, 103.4, 96.1, 77.0, 76.7, 73.9, 70.6, 61.9, 49.0. ESI HR-MS: Calculated for C₇₀H₈₅BN₉O₂₈ [M−2H]⁻ m/z 1510.5597, found 1510.5575.

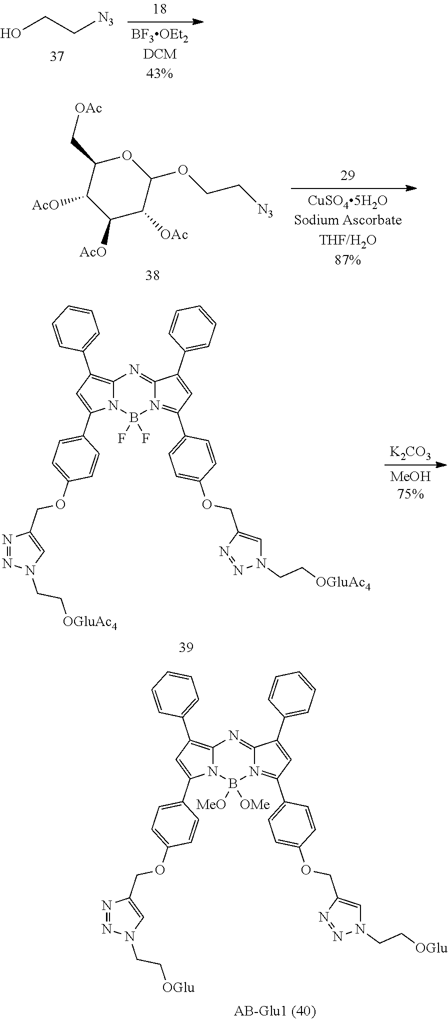

Compound 38. Compound 37 was made from 2-Bromoethanol. Briefly, to a round-bottom flask, 2-Bromoethanol (3.61 mL, 60 mmol, 1.2 equiv.) was dissolved in DI water (15 mL). Then, sodium azide (3.9 g, 50 mmol, 1 equiv.) was added, and the reaction was heated to reflux overnight. The reaction was cooled to room temperature and NaCl(s) was added until the solution was saturated. The reaction was extracted with DCM (3×), and the combine organic layers were dried with sodium sulfate and concentrated via rotary evaporation. The resulting clear, light-yellow oil 37 was used without further purification (4.3 g, 48.8 mmol, 98%). A round-bottom flask containing activated 4 Å molecular sieves was charged with 37 (0.24 g, 2.8 mmol, 1 equiv.), 18 (1.5 g, 3.04 mmol, 1.1 equiv.), and DCM (14 mL). The reaction was cooled to 0° C., and BF₃·OEt₂ (35 μL, 0.28 mmol, 0.1 equiv.) diluted with DCM (2.5 mL) was added dropwise to the reaction. The reaction was slowly warmed to room temperature and stirred overnight. The reaction was filtered over a pad of celite, concentrated via rotary evaporation, and purified via column chromatography (SiO₂, 1:1 v/v EtOAc:Hexanes) to afford the desired product (496 mg, 1.2 mmol, 43%). ¹H NMR (500 MHz, CDCl₃) δ 5.22 (t, J=9.5 Hz, 1H), 5.10 (t, J=9.7 Hz, 1H), 5.03 (dd, J=9.6, 8.0 Hz, 1H), 4.60 (d, J=7.9 Hz, 1H), 4.26 (dd, J=12.3, 4.8 Hz, 1H), 4.16 (dd, J=12.2, 2.4 Hz, 1H), 4.07-4.00 (m, 1H), 3.75-3.65 (m, 2H), 3.54-3.44 (m, 1H), 3.33-3.25 (m, 1H), 2.10-1.94 (m, 15H).

Compound 39. In a round bottom flask, 29 (75 mg, 124 μmol, 1 equiv.), 38 (206 mg, 495 μmol, 4 equiv.), and CuSO₄·5H₂O (309 mg, 1.24 mmol, 10 equiv.) were suspended in THF (6.2 mL). Sodium ascorbate (125.1 mg, 632 μmol, 5.1 equiv.) was dissolved in DI water (1.25 mL) and added dropwise to the reaction mixture. The reaction was allowed to stir under N₂ for 3 hours at room temperature. The reaction mixture was concentrated and purified via column chromatography (SiO₂, 1:19 v/v MeOH:DCM) to afford a green solid (191 mg, 133 μmol, 87%). ¹H NMR (500 MHz, CDCl₃) δ 8.07 (dt, J=21.4, 11.0 Hz, 4H), 7.74 (d, J=16.1 Hz, 1H), 7.44 (dt, J=14.8, 7.1 Hz, 3H), 7.13 (d, J=8.4 Hz, 2H), 7.05 (s, 1H), 5.29 (s, 2H), 5.19 (dt, J=21.3, 9.5 Hz, 1H), 5.13-4.97 (m, 1H), 4.60 (td, J=25.2, 22.5, 11.7 Hz, 3H), 4.47 (d, J=7.9 Hz, 1H), 4.24 (ddd, J=13.8, 9.3, 4.7 Hz, 3H), 4.19-4.09 (m, 2H), 3.70 (tdd, J=11.3, 6.5, 2.8 Hz, 3H), 2.14-1.94 (m, 25H).

AB-Glu1 (40). To a round bottom flask, 39 (190 mg, 132 μmol, 1 equiv.) and potassium carbonate (171 mg, 1.24 mmol, 9.4 equiv.) were suspended in methanol (13.2 mL). The reaction was stirred at room temperature for 2.5 hours. Then, the solvent was removed via rotary evaporation and the solid was purified via reverse-phase chromatography (C18, 100% H₂O→50% Acetonitrile/H₂O) to afford the product as a green solid (111 mg, 99 μmol, 75%). ¹H NMR (500 MHz DMSO-d₆) δ 8.18 (s, 4H), 8.07 (s, 2H), 7.48 (t, J=23.2 Hz, 12H), 7.09 (s, 1H), 5.25 (s, 4H), 4.32 (s, 1H), 3.76 (s, 2H), 3.58 (dd, J=12.4, 5.9 Hz, 3H), 3.49-3.20 (m, 9H), 3.14 (t, J=8.7 Hz, 2H), 2.78 (s, 4H). ¹³C NMR (125 MHz, DMSO) ¹³C NMR (126 MHz, DMSO) δ 129.7, 128.7, 128.4, 125.7, 103.0, 77.0, 76.5, 73.3, 70.0, 67.4, 61.0, 49.9.

### Example 3. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound represented by Formula I: 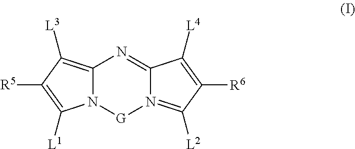
wherein
G comprises a metalloid;
L¹, L², L³, and L⁴ are independently -PhJCH₂R¹, -PhR₂, -Ph(R³)ₘ, -Ph(R⁴)ₙ, wherein Formula I includes at least one -PhJCH₂R¹;
J is O, S, or NR^{a};
R^{a} is each independently H, —(C₁-C₆)alkyl, or —(C₃-C₆) cycloalkyl;
R¹ is a substituted triazole;
R² is JCH₂R¹, H, halo, —(C₁-C₆)alkyl, —(C₃-C₆) cycloalkyl, —OR^{a}, —SR^{a}, or —N(R^{a})₂;
R³ and R⁴ are each independently H, halo, —(C₁-C₆)alkyl, —(C₃-C₆) cycloalkyl, —OR^{b}, —SR^{b}, —N(R^{b})₂, wherein R^{b} is H, —(C₁-C₆)alkyl, —(C₁-C₆)alkenyl, —(C₁-C₆)alkynyl, tris[(2-pyridyl)methyl]amine (TPA), or a metal coordination complex of TPA;
R⁵ and R⁶ are independently H, halo, —(C₁-C₆)alkyl, —(C₃-C₆) cycloalkyl, —OR^{a}, —SR^{a}, or —N(R^{a})₂; and
m and n are independently 1, 2 or 3.

2. The compound of claim 1 wherein G is BX₂ wherein X is halo or alkoxy.

3. The compound of claim 1 wherein the substituted triazole comprises one or more saccharides, polyols, or a combination thereof.

4. The compound of claim 3 wherein the one or more saccharides comprise glucose.

5. The compound of claim 3 wherein the one or more polyols comprise glycerol.

6. The compound of claim 1 represented by Formula II or Formula III: 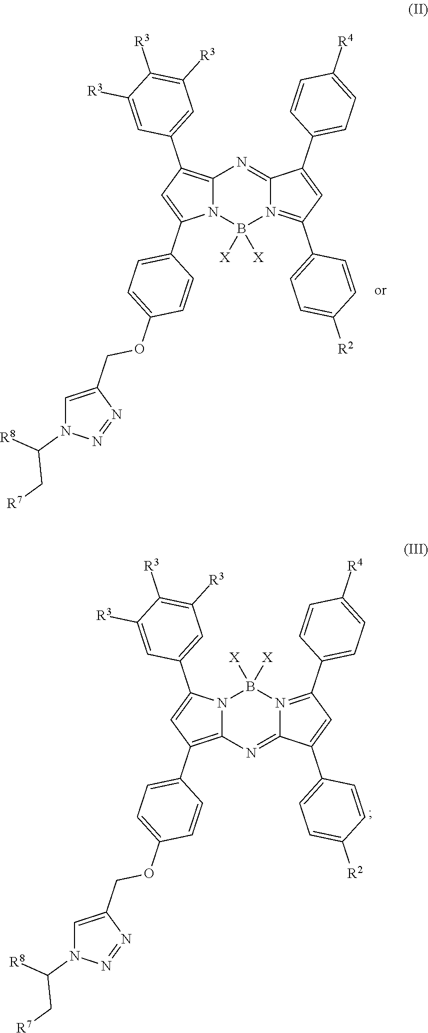
wherein
R⁷ comprises a hexose or an acyclic triol;
R⁸ is —CH₂R⁷ or H; and
X is fluoro, chloro, bromo, iodo, methoxy, ethoxy, or propoxy.

7. The compound of claim 6 wherein X is fluoro or methoxy.

8. The compound of claim 6 wherein R² is: 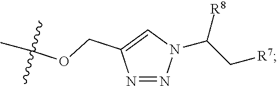
wherein
R⁷ comprises a hexose or an acyclic triol; and
R⁸ is —CH₂R⁷ or H.

9. The compound of claim 6 wherein R³ at the position para to the pyrrole moiety of Formula II or Formula III is:
H, OH, —OCH₃, 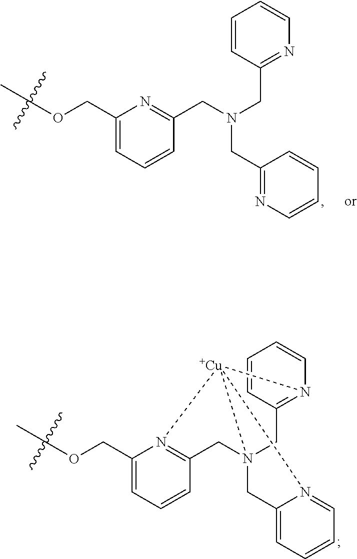
and/or
R³ at both positions meta to the pyrrole moiety of Formula II or Formula III are independently H or chloro.

10. The compound of claim 6 wherein R⁴ is —OCH₃.

11. The compound of claim 6 wherein R⁷ is: 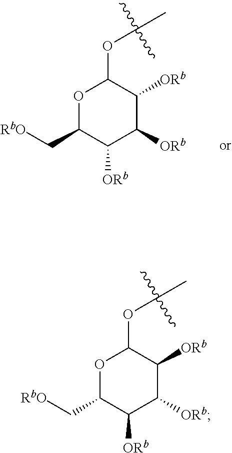
wherein each R^{b} is independently H, —(C₁-C₆)alkyl, or —CO(C₁-C₆)alkyl.

12. The compound of claim 6 wherein R⁷ is: 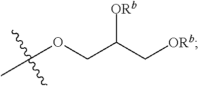
wherein each R^{b} is independently H, —(C₁-C₆)alkyl, or —CO(C₁-C₆)alkyl.

13. The compound of claim 6 wherein R⁸ is —CH₂R⁷.

14. The compound of claim 1 wherein the compound is:

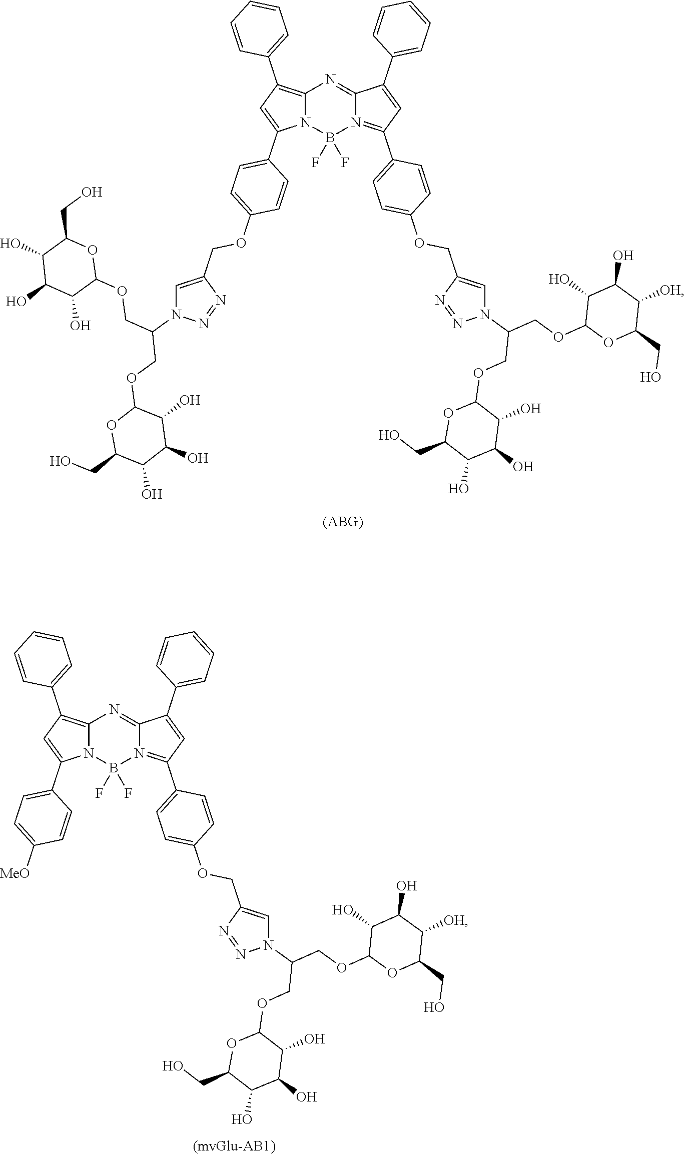

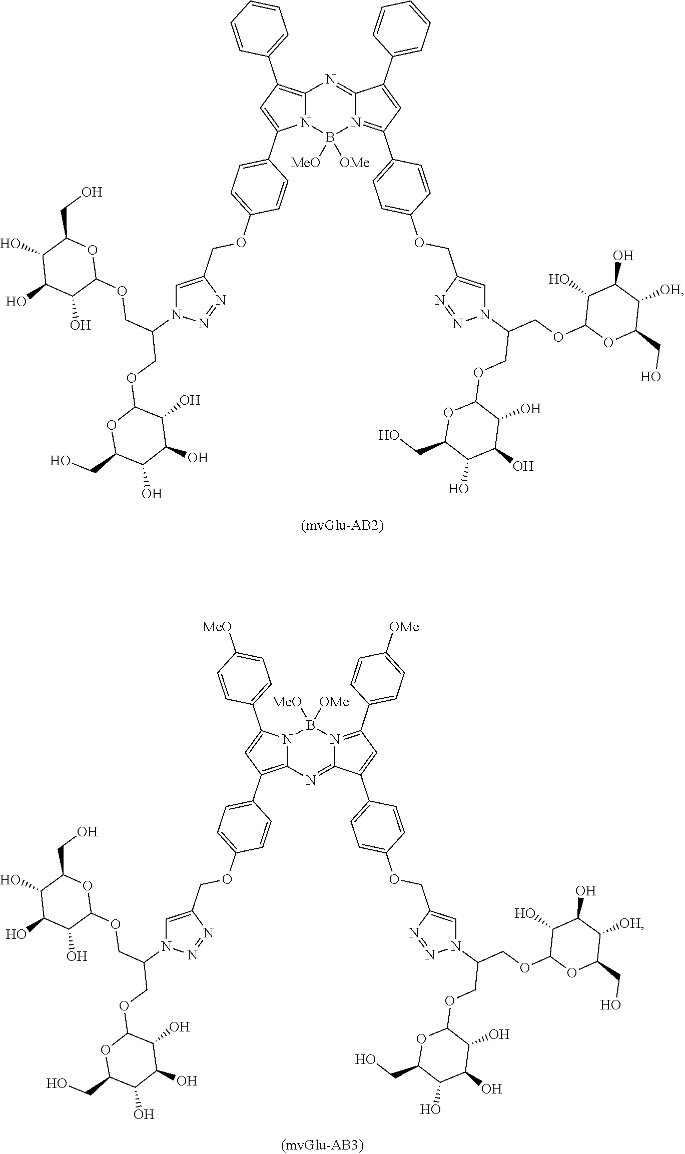

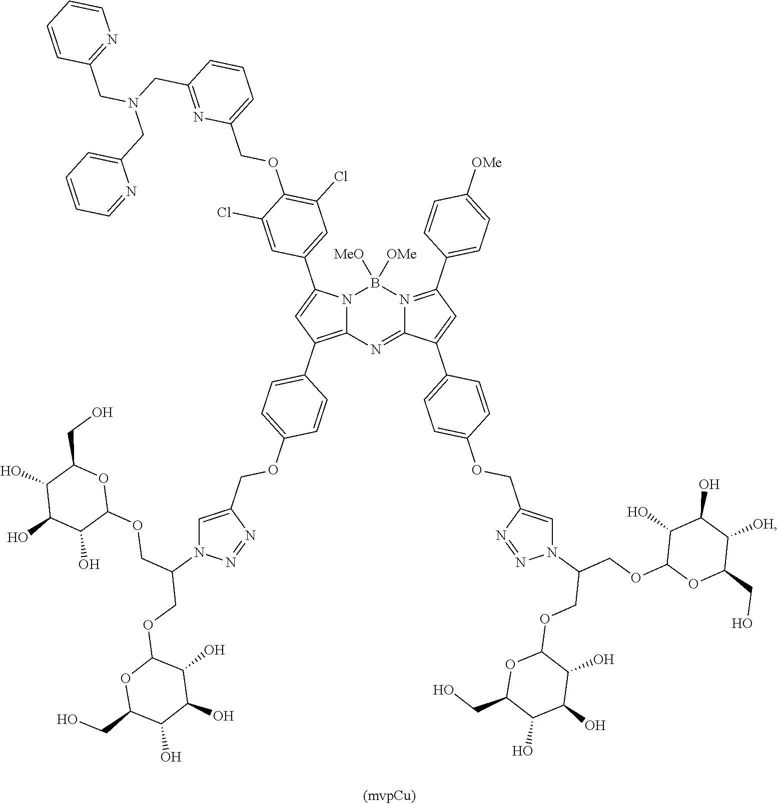

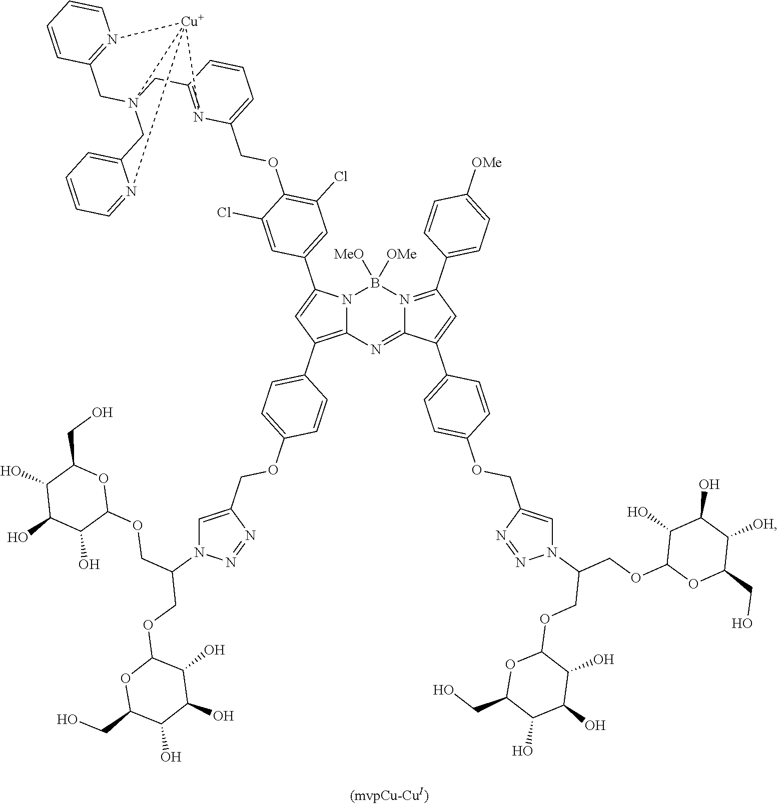

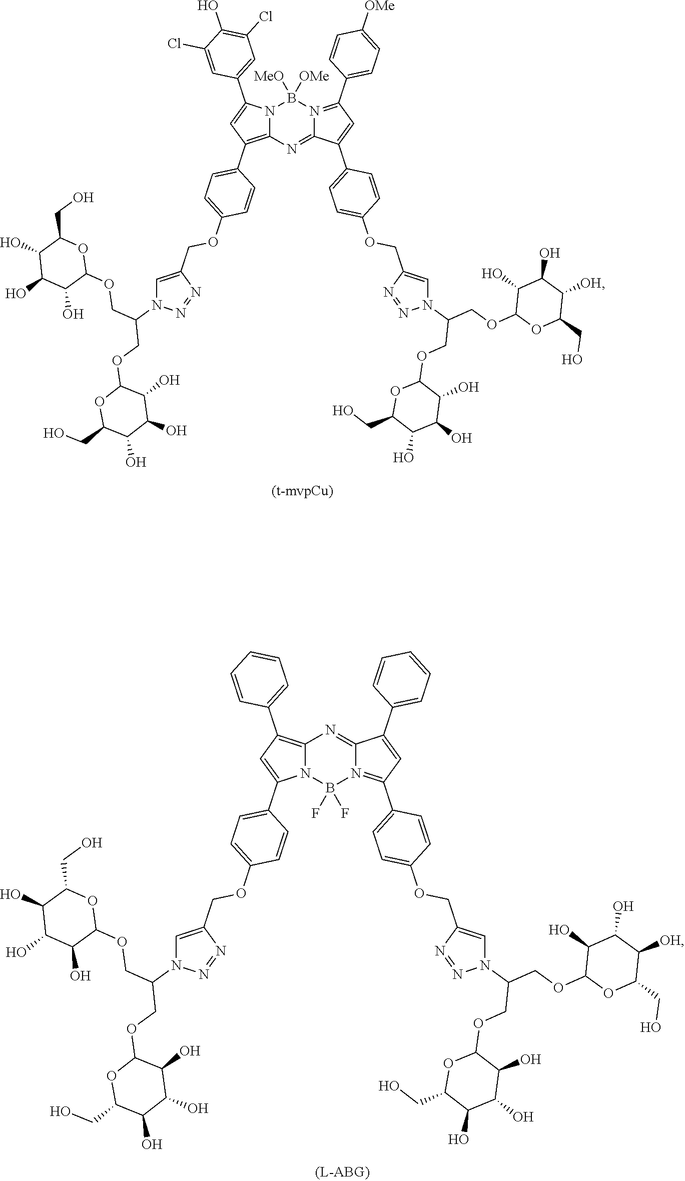

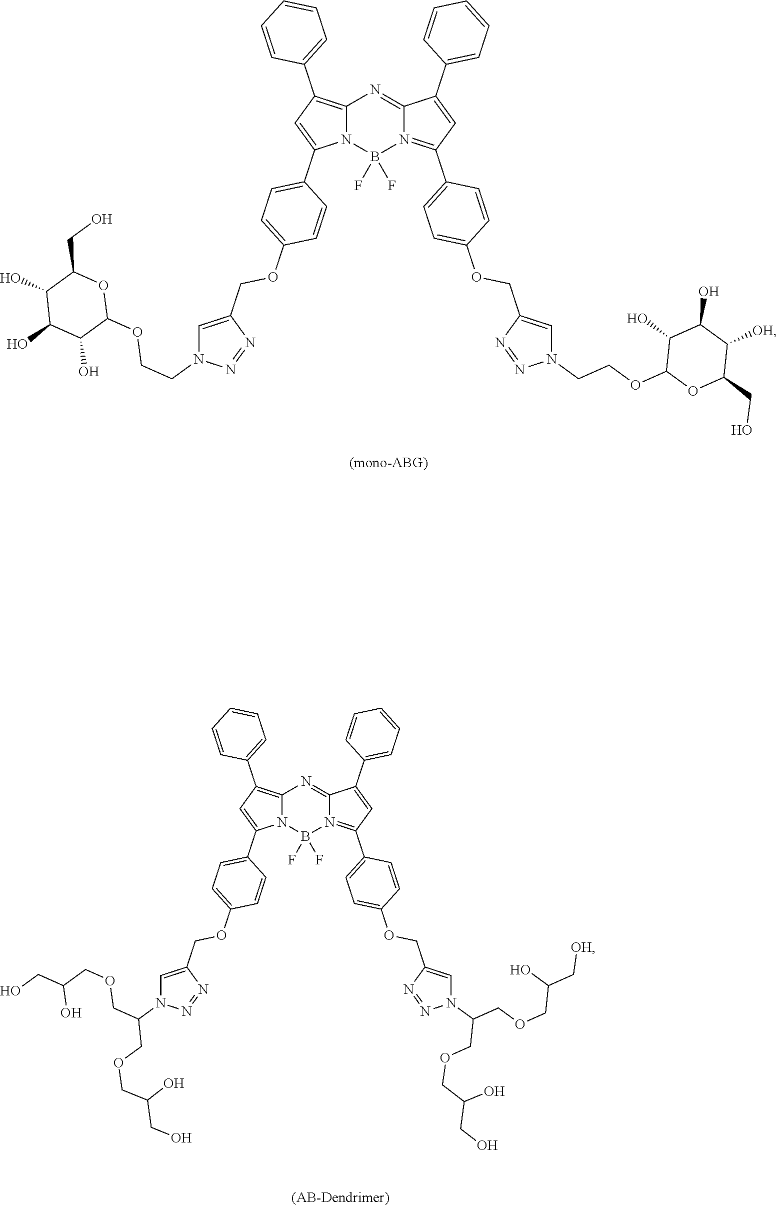

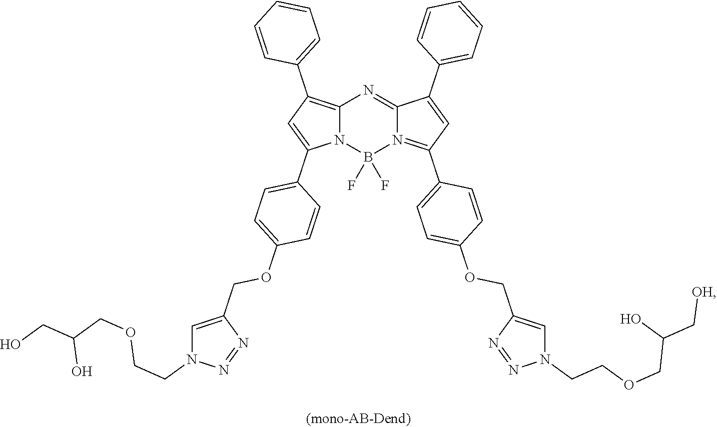

15. A method for detecting a cancer comprising:
a) contacting cancer cells and a compound of claim 1 in-vivo or in-vitro, wherein the compound binds to the cancer cells to form a bound compound;
b) irradiating the bound compound with near infrared (NIR) radiation; and
c) detecting a fluorescent signal or photoacoustic signal from the bound compound;
wherein a fluorescent signal or photoacoustic signal is emitted from the bound compound and the cancer is thereby detected.
